(19) United States
(12) Patent Application Publication
(10) Pub. No.: US 2007/0099962 A1
(43) Pub. Date: May 3, 2007

(54) 5,6-Dihydropyrin-2-one compounds useful as inhibitors of thrombin

(76) Inventors: Kristina Berggren, Molndal (SE); Ojvind Davidsson, Molndal (SE); Ola Fjellstrom, Molndal (SE); David Gustafsson, Molndal (SE); Stephen Hanessian, Montreal (CA); Tord Inghardt, Molndal (SE); Mats Nagard, Molndal (SE); Ingemar Nilsson, Molndal (SE); Eric Therrien, Montreal (CA); Willem Van Otterlo, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/596,506

(22) PCT Filed: Dec. 15, 2004
(86) PCT No.: PCT/SE04/01878

§ 371 Date: Nov. 17, 2006

(30) Foreign Application Priority Data
Dec. 18, 2003 (SE) 0303452-7
May 25, 2004 (SE) 0401344-7

(51) Int. Cl. C07D 401/02; C07D 213/63; C07D 213/84; A61K 31/4439; A61K 31/4412

(52) U.S. Cl. 514/340; 514/344; 514/345; 546/268.1; 546/288; 546/291; 546/297

(57) ABSTRACT

There is provided a compound of formula (I) wherein R¹, R^{2a}, R^{2b}, R^{3a}, R^{3b}, R⁴, R⁵, R⁶, A, G and L have meanings given in the description, which compounds are useful as, or are useful as prodrugs of, competitive inhibitors of trypsin-like proteases, such as trombin, and thus, in particular, in the treatment of conditions where inhibition of thrombin is beneficial (e.g. conditions, such as thrombo-embolisms, where inhibition of trombin is required or desired, and/or conditions whereas anticoagulant therapy is indicated).

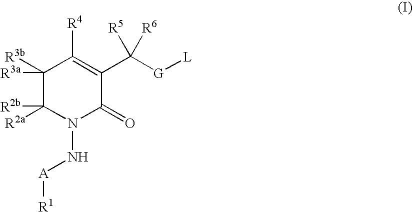

### FIELD OF THE INVENTION

This invention relates to novel pharmaceutically useful compounds, in particular compounds that are, and/or compounds that are metabolised to compounds which are, competitive inhibitors of trypsin-like serine proteases, especially thrombin, their use as medicaments, pharmaceutical compositions containing them and synthetic routes to their production.

1. Background

Blood coagulation is the key process involved in both haemostasis (i.e. the prevention of blood loss from a damaged vessel) and thrombosis (i.e. the formation of a blood clot in a blood vessel, sometimes leading to vessel obstruction).

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V, factor VIII and FXI leading to a “positive feedback” generation of thrombin from prothrombin.

By inhibiting the aggregation of platelets and the formation and crosslinking of fibrin, effective inhibitors of thrombin would be expected to exhibit antithrombotic activity. In addition, antithrombotic activity would be expected to be enhanced by effective inhibition of the positive feedback mechanism. Indeed, the convincing antithrombotic effects of a thrombin inhibitor in man has recently been described by S. Schulman et al. in N. Engl. J. Med. 349, 1713-1721 (2003).

2. Prior Art

The early development of low molecular weight inhibitors of thrombin has been described by Claesson in Blood Coagul. Fibrinol. 5, 411 (1994).

Blombäck et al. (in J. Clin. Lab. Invest. 24, suppl. 107, 59 (1969)) reported thrombin inhibitors based on the amino acid sequence situated around the cleavage site for the fibrinogen Aα chain. Of the amino acid sequences discussed, these authors suggested the tripeptide sequence Phe-Val-Arg (P9-P2-P1, hereinafter referred to as the P3-P2-P1 sequence) would be the most effective inhibitor.

Thrombin inhibitors based on peptidyl derivatives, having cyclic or acyclic basic groups at the P1-position (e.g. groups containing amino, amidino or guanidino functions), are disclosed in, for example, International Patent Application numbers WO 93/11152, WO 93/18060, WO 94/29336, WO 95/23609, WO 95/35309, WO 96/03374, WO 96/25426, WO 96/31504, WO 96/32110, WO 97/02284, WO 97/23499, WO 97/46577, WO 97/49404, WO 98/06740, WO 98/57932, WO 99/29664, WO 00/35869, WO 00/42059, WO 01/87879, WO 02/14270, WO 02/44145 and WO 03/018551, European Patent Application numbers 185 390, 468 231, 526 877, 542 525, 559 046 and 641 779, 648 780, 669 317 and U.S. Pat. No. 4,346,078.

Inhibitors of serine proteases (e.g. thrombin) based on electrophilic ketones in the P1-position are also known, such as the compounds disclosed in European Patent Application numbers 195 212, 362 002, 364 344 and 530 167.

Inhibitors of trypsin-like serine proteases based on C-terminal boronic acid derivatives of arginine (and isothiouronium analogues thereof) are known from European Patent Application number 293 881.

Achiral thrombin inhibitors having, at the P2-position of the molecule, a phenyl group, and a cyclic or acyclic basic group at the P3-position, are disclosed in International Patent Application numbers WO 94/20467, WO 96/06832, WO 96/06849, WO 97/11693, WO 97/24135, WO 98/01422 and WO 01/68605, as well as in Bioorg. Med. Chem. Lett. 7, 1283 (1997).

International Patent Application numbers WO 99/26920 and WO 0179155 disclose thrombin inhibitors having groups at the P2-position based, respectively, upon 2-aminophenols and 1,4-benzoquinones. Similar, phenol-based compounds are also disclosed in International Patent Application number WO 01/68605.

Further known inhibitors of thrombin and other trypsin-like serine proteases are based (at the P2-position of the molecule) on the 3-amino-2-pyridone structural unit. For example, compounds based upon 3-amino-2-pyridone, 3-amino-2-pyrazinone, 5-amino-6-pyrimidone, 5-amino-2,6-pyrimidione and 5-amino-1,3,4-triazin-6-one are disclosed in International Patent Application numbers WO 96/18644, WO 97/01338, WO 97/30708, WO 98/16547, WO 99/26926, WO 00/73302, WO 00/75134, WO 01/38323, WO 01/04117, WO 01/70229, WO 01/79262, WO 02/057225, WO 02/064140 and WO 03/29224, U.S. Pat. Nos. 5,668,289 and 5,792,779, as well as in Bioorg. Med. Chem. Lett. 8, 817 (1998), ibid. 13, 161 (2003) and J. Med. Chem. 41, 4466 (1998).

Thrombin inhibitors based upon 2-oxo-3-amino-substituted saturated azaheterocycles are disclosed in International Patent Application number WO 95/35313. More recently, thrombin inhibitors have been disclosed that are based upon 4-amino-3-morpholinone (see J. Med. Chem. 46, 1165 (2003)).

None of the above-mentioned documents disclose or suggest compounds based (at the P2-position) on the 1-amino-2-oxo-1,2,5,6-tetrahydropyridine is structural unit.

Moreover, there remains a need for effective inhibitors of trypsin-like serine proteases, such as thrombin. There is also a need for compounds that have a favourable pharmacokinetic profile. Such would be expected to be useful as anticoagulants and therefore in the therapeutic treatment of thrombosis and related disorders.

### DISCLOSURE OF THE INVENTION

According to the invention there is provided a compound of formula I 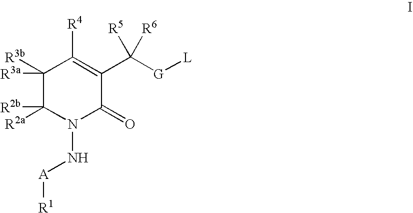
wherein
A represents C(O), S(O)₂, C(O)O (in which latter group the 0 moiety is attached to R¹), C(O)NH, S(O)₂NH (in which latter two groups the NH moiety is attached to R¹) or C₁₋₆ alkylene;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{7a}, S(O)ₙR^{7b}, S(O)₂N(R^{7c})(R^{7d}), N(R^{7e})S(O)₂R^{7f}, N(R^{7g}), B¹—C(O)—B²—R⁷ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ allyl, C₁₋₆ alkoxy and aryl), OR^{7a}, S(O)ₙR^{7b}, S(O)₂N(R^{7c})(R^{7d}), N(R^{7e})S(O)₂R^{7f}, N(R^{7g})(R^{7h}), B³—C(O)—B⁴—R⁷ⁱ, aryl and Het²,
(c) aryl, or
(d) Het³;
   R^{7a} to R⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
   provided that R^{b} does not represent H when n is 1 or 2;
   R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene;
   R⁴ represents
(a) H,
(b) halo,
(c) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl),
(d) together with R⁵, R⁴ represents C₂₋₃ n-alkylene, T¹-(C₁₋₂ n-alkylene) or (C₁₋₂ n-alkylene)-T¹, which latter three groups are optionally substituted by halo, or
(e) together with R⁵ and R⁶, R⁴ represents T²-[C(H)═], wherein T² is bonded to the C-atom to which the group R⁴ is attached;
   R⁵ and R⁶ independently represent H, F or methyl (which latter group is optionally substituted by one or more F atoms), or
(a) together with R⁴, R⁵ represents C₂₋₃ n-alkylene, T¹-(C₁₋₂ n-alkylene) or (C₁₋₂ n-alkylene)-T¹, which latter three groups are optionally substituted by halo, or
(b) together with R⁴, R⁵ and R⁶ represents T²-[C(H)═], wherein T² is bonded to the C-atom to which the group R⁴ is attached;
   T¹ and T² independently represent O, S, N(H) or N(C₁₋₄ alkyl);
   G represents
   (a) —C(O)N(R^{8a})—[CH(C(O)R⁹)]₀₋₁—C₀₋₃ alkylene-(Q¹)ₐ-,
   (b) —C(O)N(R^{8b})—C₂₋₃ alkenylene-(Q¹)ₐ-, 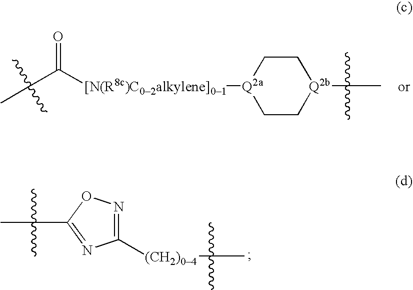
      R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl;
      Q¹ represents O, NR^{10a}, [N(H)]₀₋₁C(O)—C₀₋₂ alkylene, C(O)NHNHC(O), or —N═C(R^{10b})—;
      a represents 0 or 1;
      Q^{2a} represents 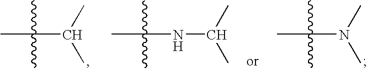
      Q^{2b} represents 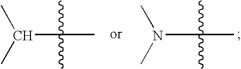
      L represents
   (a) C₀₋₆ alkylene-R^{a},
   (b) C₀₋₂ alkylene-CH═CH—C₀₋₂ alkylene-R^{a},
   (c) C₀₋₂ alkylene-C≡C—C₀₋₂ alkylene-R^{a}, 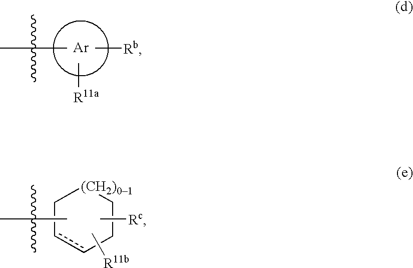
   wherein the dashed line represents an optional double bond, or 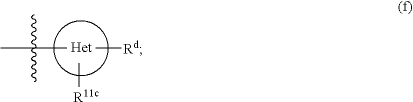
      Ar represents phenyl or naphthyl;
      Het represents a 5- to 10-membered heterocyclic group comprising one or two rings and containing, as heteroatom(s), one sulfur or oxygen atom and/or one or more nitrogen atoms;
      R^{11a} represents H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}) and S(O)₀₋₂R^{12d};
      R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}), S(O)₀₋₂R^{12d}, ═O, ═NH, ═NOH and ═N—CN;
      R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
      R^{12d} represents, independently at each occurrence, C₁₋₆ alkyl optionally substituted by one OH or N(R^{12e})R^{12f} group or by one or more halo atoms;
      R^{12e} and R^{12f} represent, independently at each occurrence, H or C₁₋₄ alkyl optionally substituted by one or more halo atoms;
      R^{a} to R^{d} independently represent 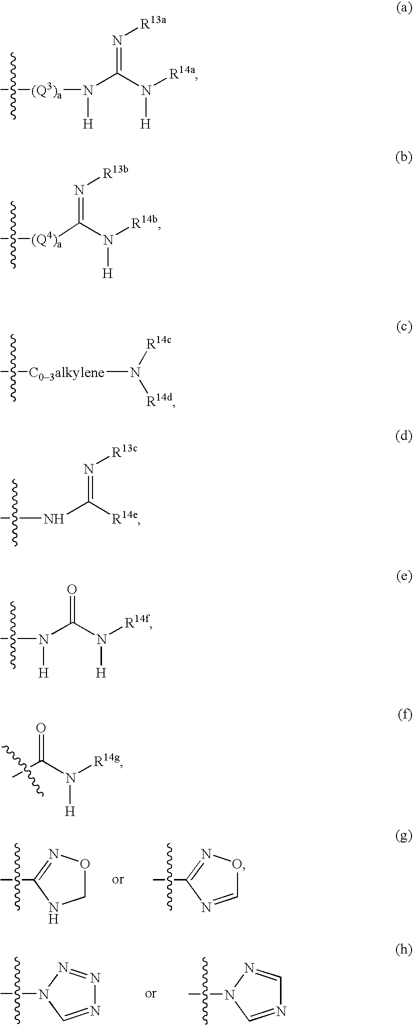
      or R^{b} to R^{d} may also represent H;
      Q³ represents O, N(R^{10c}), S(O)₂, S(O)₂NH, C(O) or —CH═N—;
      Q⁴ represents O, S or CH₂;
      a represents 0 or 1;
      R^{13a} to R^{13c} independently represent
   (a) H,
   (b) CN,
   (c) NH₂,
   (d) OR¹⁵ or
   (e) C(O)OR¹⁶;
      R¹⁵ represents
   (a) H,
   (b) C₁₋₁₀ alkyl, C₃₋₁₀ alkenyl, C₃₋₁₀ alkynyl,
   (c) C₃₋₁₀ cycloalkyl, C₄₋₁₀ cycloalkenyl, which latter two groups are optionally substituted by one or more substituents selected from halo and C₁₋₆ alkyl, or
   (d) C₁₋₃ alkyl, which latter group is optionally interrupted by oxygen and is substituted by aryl or —O-aryl;
      R¹⁶ represents
   (a) C₁₋₁₀ alkyl, C₃₋₁₀ alkenyl, C₃₋₁₀ alkynyl, which latter three groups are optionally interrupted by one or more oxygen atoms, or
   (b) C₃₋₁₀ cycloalkyl, C₄₋₁₀ cycloalkenyl, which latter two groups are optionally substituted by one or more substituents selected from halo and C₁₋₆ alkyl, or
   (c) C₁₋₃ alkyl, which latter group is optionally interrupted by oxygen and is substituted by aryl or —O-aryl;
      R^{8a} to R^{8c}, R^{10a} to R^{10c} and R^{14a} to R^{14g} independently represent
   (a) H or
   (b) C₁₋₄ alkyl (which latter group is optionally substituted by one or more substituents selected from halo and OH),
      or R^{14a} and R^{14b} independently represent C(O)O—C₁₋₆ alkyl (the alkyl part of which latter group is optionally substituted by aryl and/or one or more halo atoms),
      or R^{14c} represents
   (a) C₁₋₄ allyl substituted by C₃₋₇ cycloalkyl or aryl,
   (b) C₃₋₇ cycloalkyl,
   (c) C(O)O—C₁₋₆ alkyl (the alkyl part of which latter group is optionally substituted by aryl and/or one or more halo atoms),
   (d) C(O)C₁₋₁₆ alkyl,
   (e) C(O)N(H)—C₁₋₆ alkyl (the alkyl part of which latter group is optionally substituted by aryl and/or one or more halo atoms) or
   (f) S(O)₂—C₁₋₆ alkyl (the alkyl part of which latter group is optionally substituted by aryl and/or one or more halo atoms),
      or R^{14c} and R^{14d} together represent C₃₋₆ n-alkylene optionally interrupted by O, S, N(H) or N(C₁₋₄ alkyl) and/or substituted by one or more C₁₋₄ alkyl groups;
      each aryl independently represents a C₆₋₁₀ carbocyclic aromatic group, which group may comprise either one or two rings and may be substituted by one or more substituents selected from
   (a) halo,
   (b) CN,
   (c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
   (d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
   (e) OR^{17a},
   (f) S(O)ₚR^{17b},
   (g) S(O)₂N(R^{17c})(R^{17d})
   (h) N(R^{17e})S(O)₂R^{17f},
   (i) N(R^{17g})(R^{17h}),
   (j) B⁵—C(O)—B⁶—R^{l7i},
   (k) phenyl (which latter group is optionally substituted by halo),
   (l) Het⁹ and
   (m) Si(R^{18a})(R^{18b})(R^{18c});
      R^{17a} to R¹⁷ⁱ independently represent, at each occurrence,
   (a) H,
   (b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
   (c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
   (d) phenyl (which latter group is optionally substituted by halo) or
   (e) Het¹²,
      provided that R^{17b} does not represent H when p is 1 or 2;
      Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
   (a) halo,
   (b) CN,
   (c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
   (d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
   (e) ═O,
   (f) OR^{19a},
   (g) S(O)_{q}R^{19b},
   (h) S(O)₂N(R^{19c})(R^{19d}),
   (i) N(R^{19e})S(O)₂R^{19f},
   (j) N(R^{19g})(R^{19h}),
   (k) B⁷—C(O)—B⁸—R¹⁹ⁱ,
   (l) phenyl (which latter group is optionally substituted by halo),
   (m) Het^{c} and
   (n) Si(R^{20a})(R^{20b})(R^{20c});
      R^{19a} to R¹⁹ⁱ independently represent, at each occurrence,
   (a) H,
   (b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
   (c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
   (d) phenyl (which latter group is optionally substituted by halo) or
   (e) Het^{f},
      provided that R^{19b} does not represent H when q is 1 or 2;
      Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
      B¹ to B⁸ independently represent a direct bond, O, S or NH;
      n, p and q independently represent 0, 1 or 2;
      R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl);
      unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
   or a pharmaceutically-acceptable derivative thereof,
   which compounds are referred to hereinafter as “the compounds of the invention”.

The term “pharmaceutically-acceptable derivatives” includes pharmaceutically-acceptable salts (e.g. acid addition salts).

For the avoidance of doubt, the definitions of the terms aryl, alkyl, alkenyl, alkynyl, cycloalkyl, cycloalkenyl, alkylene, alkenylene and alkoxy groups provided above apply, unless otherwise stated, at each usage of such terms herein.

The term “halo”, when used herein, includes fluoro, chloro, bromo and iodo.

Heterocyclic (Het, Het¹ to Het¹² and Het^{a} to Het^{f}) groups may be fully saturated, partly unsaturated, wholly aromatic or partly aromatic in character. Values of heterocyclic (Het, Het¹ to Het¹² and Het^{a} to Het^{f}) groups that may be mentioned include 1-azabicyclo[2.2.2]octanyl, benzimidazolyl, benzo[c]isoxazolidinyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazolidinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo-[c]furanyl, 1,3-dihydro-2,1-benzisoxazolyl 2,3-dihydropyrrolo[2,3-b]-pyridinyl, dioxanyl, furanyl, hexahydropyrimidinyl, hydantoinyl, imidazolyl, imidazo[1,2-a]pyridinyl, imidazo[2,3-b]thiazolyl, indolyl, isoquinolinyl, isoxazolidinyl, isoxazolyl, maleimido, morpholinyl, naphtho[1,2-b]furanyl, oxadiazolyl, 1,2- or 1,3-oxazinanyl, oxazolyl, phthalazinyl, piperazinyl, piperidinyl, purinyl, pyranyl, pyrazinyl, pyrazolyl, pyridinyl, pyrimidinyl, pyrrolidinonyl, pyrrolidinyl, pyrrolinyl, pyrrolo[2,3-b]pyridinyl, pyrrolo[5,1-b]pyridinyl, pyrrolo[2,3-c]pyridinyl, pyrrolyl, quinazolinyl, quinolinyl, sulfolanyl, 3-sulfolenyl, 4,5,6,7-tetrahydrobenzimidazolyl, 4,5,6,7-tetrahydrobenzopyrazolyl, 5,6,7,8-tetrahydrobenzo-[e]pyrimidine, tetrahydrofuranyl, tetrahydropyranyl, 3,4,5,6-tetrahydropyridinyl, 1,2,3,4-tetrahydropyrimidinyl, 3,4,5,6-tetrahydropyrimidinyl, thiadiazolyl, thiazolidinyl, thiazolyl, thienyl, thieno[5,1-c]pyridinyl, thiochromanyl, triazolyl, 1,3,4-triazolo[2,3-b]pyrimidinyl, xanthenyl and the like.

Values of Het that may be mentioned include 1-azabicyclo[2.2.2]octanyl, benzimidazolyl, benzo[c]isoxazolidinyl, benzisoxazolyl, benzo[b]furanyl, benzopyrazolyl, benzo[e]pyrimidine, benzothiazolyl, benzo[b]thienyl, benzotriazolyl, 2-oxo-2,3-dihydrobenzimidazolyl, 1,3-dihydro-2,1-benzisoxazolyl, 2,3-dihydropyrrolo[2,3-b]pyridinyl, furanyl, 2-iminohexahydropyrimidinyl, imidazolyl, imidazo[1,2-a]pyridinyl, indolyl, isoquinolinyl, isoxazolidinyl, isoxazolyl, 1,2,4-oxadiazolyl, 1,3,4-oxadiazolyl, 1,2-oxazinanyl, 2-imino-1,3-oxazinanyl, piperazinyl, piperidinyl, 2-oxo-piperidinyl, pyrazinyl, pyridinyl, pyrimidinyl, 2-iminopyrrolidinyl, 3-pyrrolinyl, pyrrolo[2,3-b]pyridinyl, pyrrolo[5,1-b]pyridinyl, pyrrolo[2,3-c]pyridinyl, pyrrolyl, quinolinyl, 4,5,6,7-tetrahydrobenzimidazolyl, 4,5,6,7-tetrahydrobenzopyrazolyl, 5,6,7,8-tetrahydrobenzo[e]-pyrimidine, 3,4,5,6-tetrahydro-pyridinyl, 3,4,5,6-tetrahydropyrimidinyl, 2-imino-thiazolidinyl, thiazolyl, thienyl and thieno[5,1-c]pyridinyl.

Values of Het¹ that may be mentioned include benzodioxolyl, benzo[b]furanyl, 2,3-dihydrobenzo[b]furanyl, pyridinyl, pyrimidinyl and thienyl.

Values of Het³ that may be mentioned include benzodioxanyl, benzo[b]dioxepanyl, 2,1,3-benzoxadiazolyl, 2-oxo-benzoxazolidinyl, benzopyrazolyl, 2,1,3-benzothiadiazolyl, benzo[b]thienyl, 2-oxochromenyl, 2,3-dihydrobenzo[b]furanyl, 1-oxo-1,3-dihydrobenzo[c]furanyl, furanyl, imidazolyl, imidazo[2,3-b]thiazolyl, isoquinolinyl, isoxazolyl, naphtho[1,2-b]furanyl, pyrazolyl, pyridinyl, pyrrolyl, quinolinyl, sulfolanyl, 3-sulfolenyl, 2,4-dioxo-1,2,3,4-tetrahydropyrimidinyl, thiazolyl, thienyl, 1,3,4-triazolo[2,3-b]pyrimidinyl and xanthenyl.

Values of Het⁹ that may be mentioned include 1,3,4-oxadiazolyl, oxazolyl and pyrazolyl.

Values of Het⁹ that may be mentioned include isoxazolyl, oxazolyl and pyridinyl.

Substituents on heterocyclic (Het, Het¹ to Het¹² and Het^{a} to Het^{f}) groups may, where appropriate, be located on any atom in the ring system including a heteroatom. The point of attachment of heterocyclic (Het, Het¹ to Het¹² and Het^{a} to Het^{f}) groups may be via any atom in the ring system including (where appropriate) a heteroatom, or an atom on any fused carbocyclic ring that may be present as part of the ring system.

For the avoidance of doubt, cycloalkyl and cycloalkenyl groups may be monocyclic or, where the number of C-atoms allows, be bi- or tri-cyclic (although monocyclic cycloalkyl and cycloalkenyl are preferred). Further, when a cycloalkyl or cycloalkenyl group is fused to two phenyl groups, the phenyl groups may also be fused to each other (to form a fused tricyclic ring system).

Compounds of formula I may exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Abbreviations are listed at the end of this specification. The wavy lines on the bonds in structural fragments signify the bond positions of those fragments.

Compounds of formula I that may be mentioned include those in which:
(1) R^{2a}, R^{2b}, R^{3a} and R^{3b} independently represent H, methyl or F;
(2) R⁴ represents
   (a) H,
   (b) halo,
   (c) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl) or
   (d) together with R⁵, R⁴ represents C₂₋₃ n-alkylene or O—(C₁₋₂ n-alkylene), which latter two groups are optionally substituted by halo and wherein the O-atom of the latter group is bonded to the C-atom to which the group R⁴ is attached;
(3) R⁵ and R⁶ independently represent H, F or methyl, or R⁵, together with R⁴, represents C₂₋₃ n-alkylene or O—(C₁₋₂ n-alkylene), which latter two groups are optionally substituted by halo and wherein the O-atom of the latter group is bonded to the C-atom to which the group R⁴ is attached;
(4) R^{11a} represents H or one or more substituents selected from halo, OH, CN, C₁₋₆ alkyl and C₁₋₆ alkoxy (which latter two groups are optionally substituted by one or more substituents selected from halo, OH, C₁₋₄ alkoxy, C(O)OR^{12a} and C(O)N(R^{12b})R^{12c});
(5) R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}), ═O, ═NH, ═NOH and ═N—CN;
(6) R¹² to R^{12c} independently represent H, C₁₋₆ allyl or C₃₋₇ cycloalkyl (which latter two groups are optionally substituted by one or more halo atoms);
(7) R^{14a} to R^{14g} independently represent
   (a) H or
   (b) C₁₋₄ alkyl (which latter group is optionally substituted by one or more substituents selected from halo and OH),
   or R^{14c} represents
   (a) C₁₋₄ alkyl substituted by C₃₋₇ cycloalkyl or aryl,
   (b) C₃₋₇ cycloalkyl,
   (c) C(O)O—C₁₋₆ alkyl (the alkyl part of which latter group is optionally substituted by aryl and/or one or more halo atoms),
   (d) C(O)C₁₋₆ alkyl,
   (e) C(O)N(H)—C₁₋₆ alkyl (the alkyl part of which latter group is optionally substituted by aryl and/or one or more halo atoms) or
   (f) S(O)₂—C₁₋₆ alkyl (the alkyl part of which latter group is optionally substituted by aryl and/or one or more halo atoms),
   or R^{14c} and R^{14d} together represent C₃₋₆ n-alkylene optionally interrupted by O, S, N(H) or N(C₁₋₄ alkyl) and/or substituted by one or more C₁₋₄ alkyl groups.

Preferred values of G include:
(a) —C(O)N(R^{8a})—C₀₋₃ alkylene-;
(b) —C(O)N(R^{8a})—CH(C(O)R⁹)—C₀₋₃ alkylene-;
(c) —C(O)N(R^{8a})—C₁₋₃ alkylene-Q¹-;
(d) —C(O)N(R^{8b})—C₂₋₃ alkenylene-;

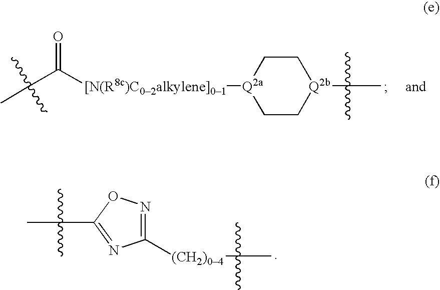

When G represents —C(O)N(R^{8a})—C₀₋₃ alkylene-Q¹-, preferred values of L include:

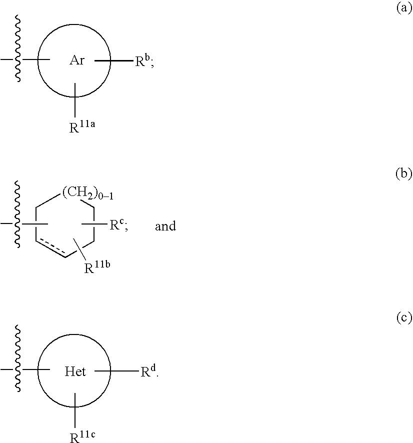

When G represents —C(O)N(R^{8b})C₂₋₃ alkenylene-, 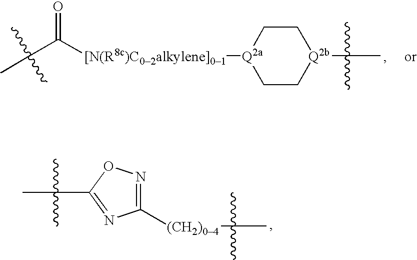
preferred values of L include:

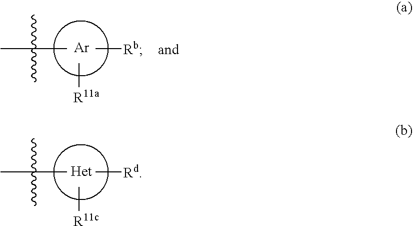

Compounds of formula I that are preferred include those in which:
(1) A represents C(O), S(O)₂, C(O)NH (in which latter group the NH moiety is attached to R¹) or C₁₋₄ alkylene;
(2) R¹ represents
   (a) C₁₋₆ alkyl, C₂₋₆ alkenyl, C₂₋₆ alkynyl (which latter three groups are optionally substituted by one or more substituents selected from halo, CN, C₃₋₈ cycloalkyl (optionally substituted by one or more substituents selected from halo, OH, ═O, C₁₋₆ alkyl, C₁₋₆ alkoxy and aryl), OR^{7a}, SR^{7b}, S(O)₂R^{7b}, S(O)₂N(H)R^{7c}, N(H)S(O)₂R^{7f}, N(R^{7g})(R^{7h}), C(O)R⁷ⁱ, OC(O)R⁷ⁱ, C(O)OR⁷ⁱ, N(H)C(O)R⁷ⁱ, C(O)N(H)R⁷ⁱ, aryl and Het¹),
   (b) C₃₋₈ cycloalkyl or C₄₋₈ cycloalkenyl, which latter two groups are optionally fused to one or two phenyl groups and are optionally substituted by one or more substituents selected from halo, ═O, C₁₋₆ alkyl, C₄₋₆ cycloalkyl (optionally substituted by one or more substituents selected from halo, C₁₋₄ alkyl, C₁₋₄ alkoxy and phenyl), OR^{7a}, SR^{7b}, S(O)₂R^{7b}, S(O)₂N(H)R^{7c}, N(H)S(O)₂R^{7f}, N(R^{7g})(R^{7h}), OC(O)R⁷ⁱ, C(O)OR⁷ⁱ, N(H)C(O)R⁷ⁱ, C(O)N(H)R⁷ⁱ, aryl and Het²,
   (c) aryl, or
   (d) Het³;
(3) R^{7a} to R⁷ⁱ independently represent, at each occurrence,
   (a) H,
   (b) C₁₋₆ alkyl, C₂₋₆ alkenyl, C₂₋₆ alkynyl (which latter three groups are optionally substituted by one or more substituents selected from halo, OH, C₁₋₄ alkoxy, aryl and Het⁴),
   (c) C₄₋₆ cycloalkyl, C₄₋₆ cycloalkenyl (which latter two groups are optionally substituted by one or more substituents selected from halo, ═O and C₁₋₄ alkyl),
   (d) aryl or
   (e) Het⁶,
   provided that R^{h} does not represent H when n is 1 or 2;
(4) R^{2a} and R^{2b} both represent H, both represent methyl or both represent F;
(5) R^{3a} and R^{3b} both represent H, both represent methyl or both represent F;
(6) R⁴ represents H, halo, C₁₋₄ alkoxy or C₁₋₄ alkyl (which latter group is optionally substituted by one or more substituents selected from halo, OH and C(O)OH (e.g. one or more substituents selected from halo and OH));
(7) R⁵ and R⁶ independently represent H or F;
(8) the group G-L takes any of the following definitions
   (a) C(O)N(R^{8a})—C₀₋₆ alkylene-R^{a},
   (b) C(O)N(R^{8a})—CH(C(O)R⁹)—C₀₋₅ alkylene-R^{a},
   (c) C(O)N(R^{8a})—C₀₋₃ alkylene-CH≡CH—C₀₋₂ alkylene-R^{a},
   (d) C(O)N(R^{8a})—C₀₋₃ alkylene-C≡C—C₀₋₂ alkylene-R^{a}, 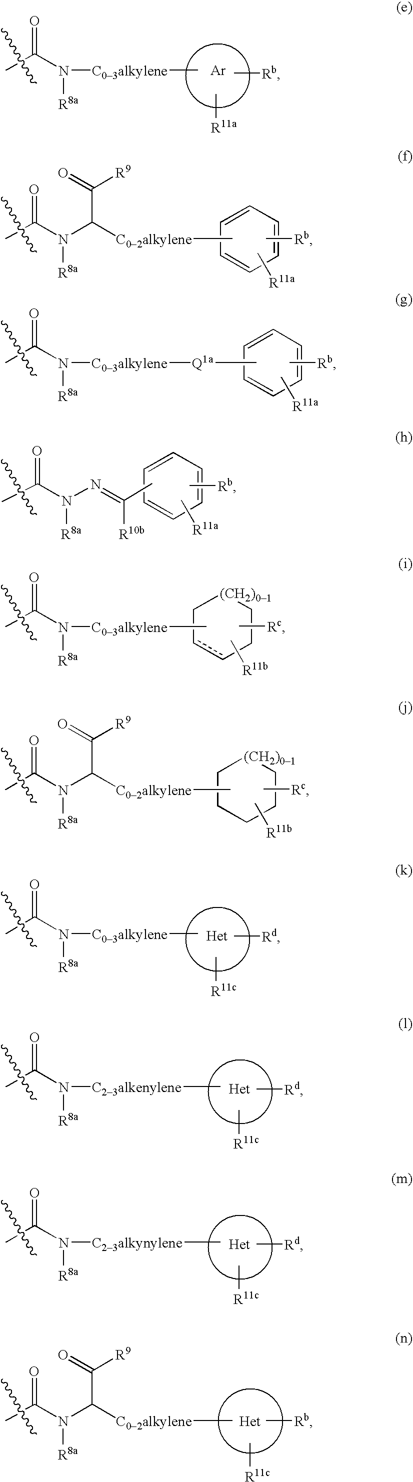 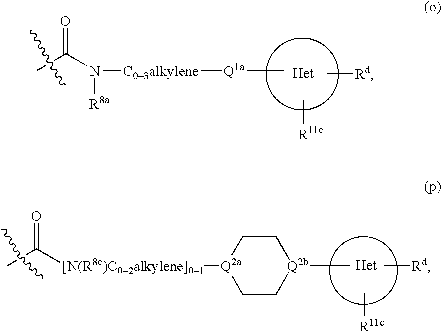
      wherein Q^{1a} represents O, NR^{10a} or [N(H)]₀₋₁C(O)—C₀₋₂ alkylene;
(9) R⁹ represents a 5- to 10-membered aromatic heterocyclic group comprising one or two rings and containing, as heteroatom(s), one sulfur or oxygen atom and/or one to three nitrogen atoms, which heterocyclic group is optionally substituted by one or more substituents selected from halo and C₁₋₄ alkyl;
(10) Het represents a 5- or 6-membered monocyclic, or a 8-, 9- or 10-membered bicyclic heterocyclic group containing, as heteroatom(s), one sulfur or oxygen atom and/or one to four nitrogen atoms;
(11) R^{11a} represents H or one to three substituents selected from halo, OH, CN, C₁₋₄ alkyl and C₁₋₄ alkoxy (which latter two groups are optionally substituted by one or more substituents selected from OH, halo, C(O)OR^{12a} and C(O)N(R^{12b})R^{12c} (e.g. one or more substituents selected from the latter three groups));
(12) R^{11b} represents H or one to three substituents selected from halo, OH, C₁₋₄ alkyl, C₁₋₄ alkoxy and ═O;
(13) R^{11c} represents H or one to three substituents selected from halo, OH, CN, C₁₋₄ alkyl, C₁₋₄ alkoxy (which latter two groups are optionally substituted by one or more substituents selected from halo, OH and C₁₋₂ alkoxy), ═O, ═NH, ═NOH and ═N—CN;
(14) R^{12a} to R^{12c} independently represent H, C₁₋₄ alkyl (optionally substituted by one N(R^{12e})R^{12f} group) or C₃₋₆ cycloalkyl (e.g. H, C₁₋₄ alkyl or C₃₋₆ cycloalkyl);
(15) R^{a} represents 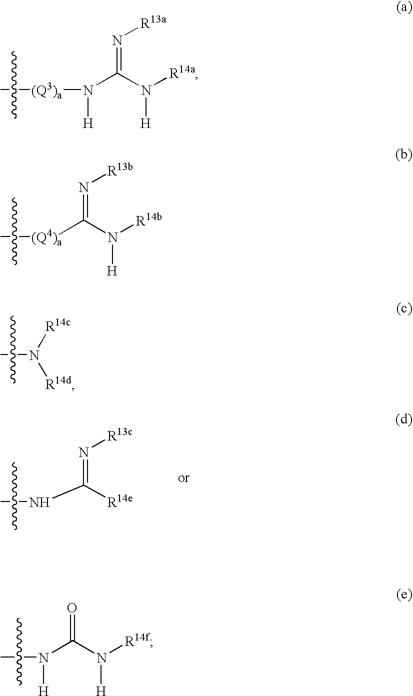
(16) R^{b} represents 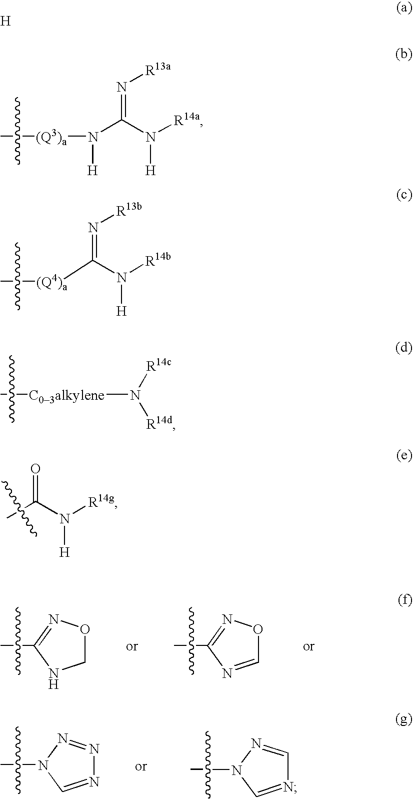
(17) R^{c} and R^{d} independently represent 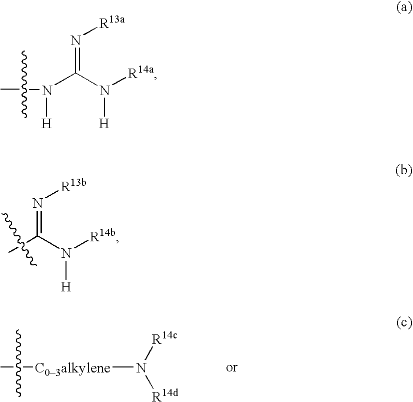
   (d) R^{d} may also represent H;
(18) Q³ represents O, S(O)₂, S(O)₂NH, C(O) or —CH═N—;
(19) Q⁴ represents O or S;
(20) R¹⁵ represents H, C₁₋₆ alkyl, C₃₋₆ alkenyl (which latter two groups are optionally interrupted by an oxygen atom), C₃₋₆ cycloalkyl or C₁₋₂ alkyl (which latter group is substituted by aryl);
(21) R¹⁶ represents C₁₋₆ alkyl, C₃₋₆ alkenyl, C₃₋₆ cycloalkyl or C₁₋₂ alkyl substituted by aryl;
(22) R^{8a} to R^{8c} represent H or methyl;
(23) R^{10a} to R^{10c} independently represent H or C₁₋₃ alkyl (which latter group is optionally substituted by OH or one or more halo atoms);
(24) R^{14a} represents C₁₋₂ alkyl, C(O)O—C₁₋₅ alkyl (the alkyl part of which latter group is optionally substituted by phenyl) or H (e.g. H or C₁₋₂ alkyl);
(25) R^{14b} to R^{14g} independently represents H or C₁₋₂ alkyl (which latter group is optionally substituted by one or more halo atoms, but is preferably unsubstituted), or R^{14c} represents C₄₋₆ cycloalkyl or C(O)O—C₁₋₅ alkyl (the alkyl part of which latter group is optionally substituted by phenyl) or R^{14c} and R^{14d} together represent C₄₋₅ n-alkylene optionally interrupted by O;
(26) each aryl independently represents phenyl or naphthyl, each of which groups may be substituted by one or more substituents selected from
   (a) halo,
   (b) CN,
   (c) C₁₋₈ alkyl, C₂₋₄ alkenyl, C₂₋₄ alkynyl (which latter three groups are optionally substituted by one or more substituents selected from halo, OH, C₁₋₂ alkoxy, C(O)OH, C(O)O—C₁₋₂ alkyl and phenyl),
   (d) C₃₋₆ cycloalkyl optionally substituted by one or more substituents selected from halo, ═O and C₁₋₄ alkyl,
   (e) OR^{17a},
   (f) SR^{17b}, S(O)₂R^{17b},
   (g) S(O)₂N(H)R^{17c},
   (h) N(H)S(O)₂R^{17f},
   (i) N(O)R^{17g},
   (j) C(O)R¹⁷ⁱ, C(O)OR¹⁷ⁱ, OC(O)R¹⁷ⁱ, C(O)N(H)R¹⁷ⁱ, N(H)C(O)R¹⁷ⁱ, N(H)C(O)OR¹⁷ⁱ,
   (k) phenyl (which latter group is optionally substituted by one or more halo atoms),
   (l) Het⁹ and
   (m) Si(CH₃)₃;
(27) R^{17a} to R¹⁷ⁱ independently represent, at each occurrence,
   (a) H,
   (b) C₁₋₈ alkyl optionally substituted by one or more substituents selected from halo, OH, C₁₋₂ alkoxy, phenyl (which latter group is optionally substituted by one or more halo atoms) and Het¹⁰ (e.g. one or more substituents selected from halo, OH, C₁₋₂ alkoxy and phenyl (which latter group is optionally substituted by one or more halo atoms)),
   (c) C₃₋₆ cycloalkyl optionally substituted by one or more substituents selected from halo, ═O and C₁₋₄ alkyl,
   (d) phenyl optionally substituted by one or more halo atoms or
   (e) Het¹²,
   provided that R^{17b} does not represent H;
(28) Het¹ to Het¹² independently represent 5- to 13-membered heterocyclic groups containing one to four heteroatoms selected from oxygen, nitrogen and/or sulfur, which heterocyclic groups may comprise one, two or three rings and may be substituted by one or more substituents selected from
   (a) halo,
   (b) CN,
   (c) C₁₋₈ alkyl, C₂₋₄ alkenyl, C₂₋₄ alkynyl (which latter three groups are optionally substituted by one or more substituents selected from halo, OH and C₁₋₂ alkoxy),
   (d) C₃₋₆ cycloalkyl optionally substituted by one or more substituents selected from halo, ═O and C₁₋₄ alkyl,
   (e) ═O,
   (f) OR^{19a},
   (g) S(O)₂R^{19b},
   (h) S(O)₂N(H)R^{19c},
   (i) N(H)S(O)₂R^{19f},
   (j) N(H)R^{19g},
   (j) C(O)R¹⁹ⁱ, C(O)OR¹⁹ⁱ, C(O)N(H)R¹⁹ⁱ, N(H)C(O)R¹⁹ⁱ, N(H)C(O)OR¹⁹ⁱ,
   (l) phenyl (which latter group is optionally substituted by halo) and
   (m) Het^{c};
(29) R^{19a} to R¹⁹ⁱ independently represent, at each occurrence,
   (a) H,
   (b) C₁₋₆ alkyl optionally substituted by one or more substituents selected from halo, OH, C₁₋₂ alkoxy and phenyl,
   (c) C₃₋₆ cycloalkyl optionally substituted by one or more substituents selected from halo, ═O and C₁₋₄ alkyl,
   (d) phenyl optionally substituted by halo or
   (e) Het^{f},
   provided that R^{19b} does not represent H;
(30) Het^{a} to Het^{f} independently represent 5- or 6-membered heterocyclic groups containing, as heteroatoms, one oxygen or sulfur atom and/or one to three nitrogen atoms, which heterocyclic groups may be substituted by one or more substituents selected from halo and C₁₋₄ alkyl.

Also preferred are compounds of formula I in which R⁵ and R⁶ both take the same definition (i.e. compounds in which R⁵ and R⁶ both represent H, both represent F or both represent methyl, CH₂F, CHF₂ or CF₃).

When A represents C(O) or C(O)NH (in which latter group the NH moiety is attached to R¹), preferred compounds of formula I also include those in which R¹ represents:
(a) C₁₋₆ alkyl, C₂₋₆ alkenyl, C₂₋₆ alkynyl, which latter three groups are
   (i) substituted by one substituent selected from C₃₋₈ cycloalkyl (optionally substituted by one or more substituents selected from halo, OH, ═O, C₁₋₆ alkyl, C₁₋₆ alkoxy and aryl), aryl and Het¹, and
   (ii) optionally substituted by one or more further substituents selected from halo, CN, C₄₋₆ cycloalkyl (optionally substituted by one or more substituents selected from halo and C₁₋₄ alkyl), OR^{7a}, SR^{7b}, S(O)₂R^{7b}, S(O)₂N(H)R^{7c}, N(H)S(O)₂R^{7f}, N(R^{7g})(R^{7h}), OC(O)R⁷ⁱ, C(O)OR⁷ⁱ, N(H)C(O)R⁷ⁱ, C(O)N(H)R⁷ⁱ, aryl and Het¹;
(b) C₃₋₈ cycloalkyl or C₄₋₈ cycloalkenyl, which latter two groups are
   (i) fused to one or two phenyl groups and optionally substituted by one or more substituents selected from halo, C₁₋₄ alkyl and C(O)OR⁷ⁱ, or
   (ii) substituted by aryl and optionally further substituted by one or more substituents selected from halo and C₁₋₄ alkyl;
(c) aryl; or
(d) Het³,
   wherein R^{7a} to R^{7c}, R^{7f} to R⁷ⁱ aryl and Het¹ are as defined above or below.

When A represents S(O)₂, preferred compounds of formula I also include those in which R¹ represents:
(a) C₁₋₃ alkyl or C₂₋₃ alkenyl, which latter two groups are substituted by aryl and are optionally further substituted by one or more halo atoms;
(b) C₁₋₆ alkyl optionally substituted by one or more substituents selected from halo, OR^{7a} and S(O)₂R^{7b};
(c) C₃₋₆ monocyclic cycloalkyl optionally substituted by one or more substituents selected from halo and C₁₋₄ alkyl;
(d) C₆₋₈ bicyclic cycloalkyl optionally substituted by one or more substituents selected from halo, ═O and C₁₋₆ alkyl;
(c) aryl; or
(d) Het³,
   wherein R^{7a} and R^{7b} are as defined above or below.

When A represents C₁₋₆ alkylene, preferred compounds of formula I also include those in which R¹ represents:
(a) C₁₋₆ alkyl or C₂₋₆ alkenyl, which latter two groups are optionally substituted by one or more substituents selected from halo and OH;
(b) C₃₋₈ cycloalkyl or C₄₋₈ (e.g. C₄₋₆) cycloalkenyl, which latter two groups are optionally substituted by one to four substituents selected from halo, ═O, OH, C₁₋₄ alkyl, O—C₁₋₄ alkyl (which latter two groups are optionally substituted by one or more halo (e.g. F) atoms) and aryl, or, particularly,
(c) aryl (e.g. naphthyl or, particularly, phenyl), or
(d) Het³,
(e.g. any one of the groups listed in (b) to (d) above).

Compounds of formula I that are more preferred include those in which the group G-L takes any of the preferred definitions provided at (8)(a), (c), (d), (e), (g), (h), (i), (k), (l), (m), (o) and (p) above.

More preferred compounds of formula I particularly include compounds in is which:
(1) A represents C(O), S(O)₂, C(O)NH (in which latter group the NH moiety is attached to R¹) or C₁₋₃ alkylene;
(2) R¹ represents
   (a) C₁₋₅ alkyl, C₂₋₄ alkenyl (which latter two groups are optionally substituted by one or more substituents selected from halo, C₆₋₈ bicyclic cycloalkyl, C₃₋₆ monocyclic cycloalkyl (which latter two groups are optionally substituted by one or more substituents selected from halo, ═O, C₁₋₄ alkyl, C₁₋₄ alkoxy and phenyl (which latter group is optionally substituted by one or more substituents selected from halo, C₁₋₄ alkyl and C₁₋₄ alkoxy)), OR^{7a}, SR^{7b}, S(O)₂R^{7b}, C(O)R⁷ⁱ, OC(O)R⁷ⁱ, C(O)OR⁷ⁱ, aryl and Het¹),
   (b) C₃₋₆ cycloalkyl or C₄₋₆ cycloalkenyl, which latter two groups are optionally fused to one or two phenyl groups and are optionally substituted by one or more substituents selected from halo, ═O, C₁₋₄ alkyl, OR^{7a}, C(O)OR⁷ⁱ and phenyl (which latter group is optionally substituted by one or more substituents selected from halo, C₁₋₄ alkyl and C₁₋₄ alkoxy),
   (c) aryl, or
   (d) Het³;
(3) R^{7a} to R⁷ⁱ independently represent, at each occurrence,
   (a) H,
   (b) C₁₋₆ alkyl, C₂₋₄ alkenyl (which latter two groups are optionally substituted by one or more substituents selected from halo, OH, C₁₋₄ alkoxy and phenyl),
   (c) C₄₋₆ cycloalkyl (which latter group is optionally substituted by one or more substituents selected from halo and C₁₋₂ alkyl) or
   (d) phenyl (which latter group is optionally substituted by one or more substituents selected from halo, C₁₋₄ alkyl and C₁₋₄ alkoxy)
   provided that R^{h} does not represent H;
(4) R^{2a} and R^{2b} both represent H;
(5) R^{3a} and R^{3b} both represent H;
(6) R⁴ represents H, halo (such as Cl) or C₁₋₃ alkyl;
(7) R⁵ and R⁶ both represent H or both represent F;
(8) the group G-L takes any of the following definitions
   (i) C(O)N(H)—C₀₋₅ alkylene-R^{a1},
   (ii) C(O)N(H)—C₀₋₃ alkylene-CH═CH—R^{a2},
   (iii) C(O)N(H)—C₁₋₃ alkylene-C≡C—CH₂—R^{a3}, 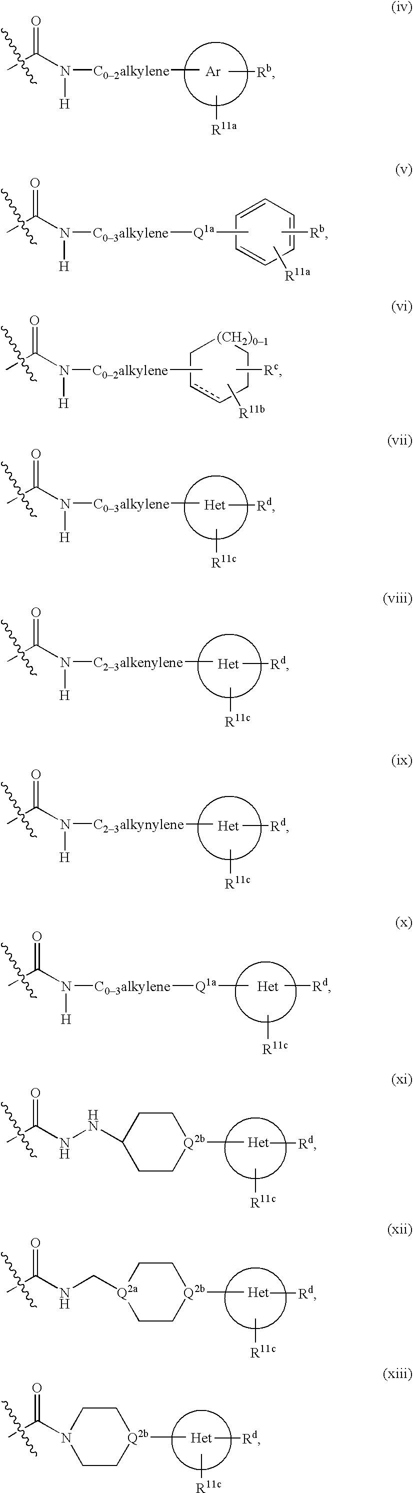
   wherein Q¹⁸ is as defined above;
(9) Het represents a 5- or 6-membered monocyclic, an 8-membered bicyclic, or a 9- or 10-membered ring-fused bicyclic heterocyclic group containing, as heteroatom(s), one sulfur or oxygen atom and/or one to three nitrogen atoms, which heterocyclic group
   (i) when 5- or 6-membered, is fully aromatic, fully saturated or mono-unsaturated,
   (ii) when 8-membered, is fully aromatic or, preferably, fully saturated, or
   (iii) when 9- or 10-membered, is fully aromatic or part-aromatic;
(10) R^{11a} represents H or one to three substituents selected from halo, OH, CN, C₁₋₃ alkyl and C₁₋₃ alkoxy (which latter two groups are optionally substituted by one or more substituents selected from OH, halo, C(O)OR^{12a} and C(O)N(R^{12b})R^{12c} (e.g. one or more substituents selected from the latter three groups));
(11) R^{11a} represents one or two substituents selected from halo and C₁₋₃ alkyl or, preferably, R^{11b} represents H;
(12) R^{11c} represents H or one to three substituents selected from halo, OH, CN, C₁₋₃ alkyl (which latter group is optionally substituted by one or more substituents selected from halo and OH), ═O, ═NH and ═N—CN;
(13) R^{12a} to R^{12c} independently represent H, C₁₋₃ alkyl (optionally substituted by one N(R^{12e})R^{12f} group) or C₃₋₅ cycloalkyl (e.g. H, C₁₋₃ alkyl or C₃₋₅ cycloalkyl);
(14) R^{12e} and R^{12f} independently represent H or C₁₋₂ alkyl;
(15) R^{a1}, R^{a2} and R^{a3} represent R^{a} as defined above, but preferably independently represent 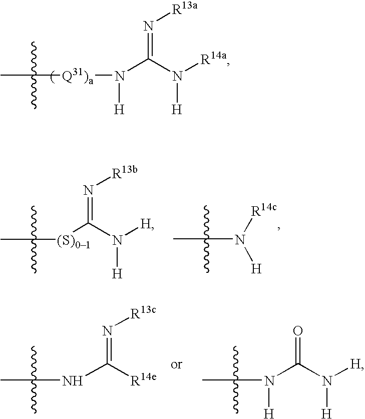
   wherein Q³¹ represents O, C(O) or —CH═N— and a represents 0 or, preferably, 1;
(16) R^{b} represents 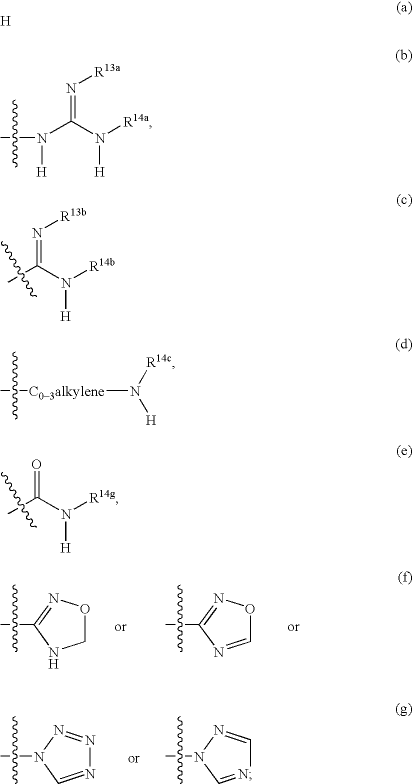
(17) R^{c} represents 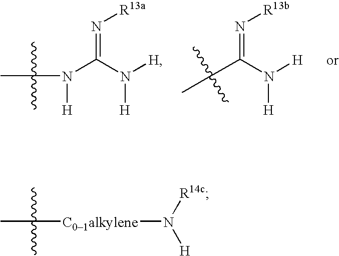
(18) R^{d} represents H, 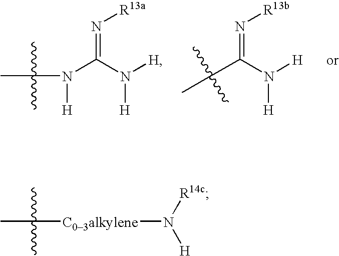
(29) R^{13a} represents H, CN, NH₂ or OR¹⁵;
(20) R^{13b} represents H, NH₂, OR¹⁵ or C(O)OR¹⁶;
(21) R^{13c} represents H or OH;
(22) R¹⁵ represents H or —C₁₋₅ alkyl;
(23) R¹⁶ represents C₁₋₂ alkyl substituted by aryl;
(24) R^{10a} represents H or C₁₋₂ alkyl (which latter group is optionally substituted by OH);
(25) R^{14a} represents H, methyl, C(O)O—C₃₋₄ alkyl or C(O)OCH₂-phenyl (e.g. methyl or, preferably, H);
(26) R^{14b} to R^{14d} and R^{14f} to R^{14g} independently represent methyl or, preferably, H,
   or R^{14c} represents
      C₁₋₂ alkyl substituted by one to three halo (e.g. F) atoms,
      C₄₋₅ cycloalkyl (e.g. cyclopentyl),
      C(O)O—C₃₋₄ alkyl or
      C(O)OCH₂-phenyl (e.g. one of the latter three groups),
   or R^{14c} and R^{14d} together represent C₄ n-alkylene;
(27) R^{14e} represents H or, preferably, methyl;
(28) each aryl independently represents phenyl or naphthyl, each of which groups may be substituted by one or more substituents selected from
   (a) F, Cl, Br,
   (b) CN,
   (c) C₁₋₆ alkyl, C₂₋₃ alkenyl (which latter two groups are optionally substituted by one or more substituents selected from F, Cl, C(O)OH, C(O)OCH₃ and phenyl),
   (d) C₃₋₅ cycloalkyl,
   (e) OR^{17a},
   (f) S—C₁₋₂ alkyl, S(O)₂—C₁₋₂ alkyl (the alkyl parts of which latter two groups are optionally substituted by one or more F atoms),
   (g) S(O)₂NH₂, S(O)₂N(H)CH₃,
   (h) N(H)S(O)₂—C₁₋₂ alkyl (the alkyl part of which latter group is optionally substituted by one or more F atoms),
   (i) NH₂, N(H)C₁₋₂ alkyl,
   (j) CHO, C(O)—C₁₋₄ alkyl (the alkyl part of which latter group is optionally substituted by one or more F or Cl atoms), C(O)OH, C(O)O—C₁₋₄ alkyl, C(O)NH₂, C(O)N(H)—C₁₋₄ alkyl, N(H)C(O)—C₁₋₄ alkyl, N(H)C(O)O—C₁₋₄ alkyl,
   (k) phenyl (which latter group is optionally substituted by one to four substituents selected from F, Cl and Br),
   (l) Het⁹ and
   (m) Si(CH₃)₃;
(29) R^{17a} represents
   (a) H,
   (b) C₁₋₅ alkyl optionally substituted by phenyl or one or more substituents selected from F and Cl,
   (c) C₃₋₅ cycloalkyl or
   (d) phenyl optionally substituted by one to four substituents selected from F, Cl and Br;
(30) Het¹ represents a 5- to 10-membered heterocyclic group containing one to three heteroatoms selected from oxygen, nitrogen and/or sulfur, which heterocyclic groups may comprise one or two rings and may be substituted by one to three substituents selected from F, Cl, Br, C₁₋₄ alkyl, ═O and OH;
(31) Het³ represents a 5- to 13-membered heterocyclic group containing one to four heteroatoms selected from oxygen, nitrogen and/or sulfur, which heterocyclic groups may comprise one, two or three rings and may be substituted by one to four substituents selected from
   (a) F, Cl, Br,
   (b) C₁₋₄ alkyl (which latter group is optionally substituted by one or more substituents selected from F, Cl and OH),
   (c) C₃₋₅ cycloalkyl,
   (d) ═O,
   (e) OH, O—C₁₋₂ alkyl (which latter group is optionally substituted by one or more substituents selected from F and Cl),
   (g) S(O)₂—C₁₋₂ alkyl (which latter group is optionally substituted by one or more F atoms),
   (h) S(O)₂NH₂, S(O)₂N(H)—C₁₋₂ alkyl,
   (i) N(H)S(O)₂—C₁₋₂ alkyl,
   (j) NH₂, N(H)—C₁₋₂ alkyl,
   (j) C(O)—C₁₋₄ alkyl, C(O)OH, C(O)O—C₁₋₄ alkyl, C(O)NH₂, C(O)N(H)—C₁₋₄ alkyl, N(H)C(O)—C₁₋₄ alkyl, N(H)C(O)O—C₁₋₄ alkyl,
   (l) phenyl (which latter group is optionally substituted by one to four substituents selected from F, Cl and Br) and
   (m) Het^{c};
(32) Het⁹ represents 5- or 6-membered monocyclic heterocyclic group containing, as heteroatom(s), one sulfur or oxygen atom and/or one to three nitrogen atoms, which heterocyclic groups may comprise one, two or three rings and may be substituted by one or more substituents selected F, Cl, Br, C₁₋₄ alkyl, ═O and OH;
(33) Het^{c} represents a 5- or 6-membered heterocyclic group containing, as heteroatoms, one oxygen atom and/or one or two nitrogen atoms, which heterocyclic groups may be substituted by one or more substituents selected from F, Cl, Br and methyl.

More preferred definitions of R^{a1} include 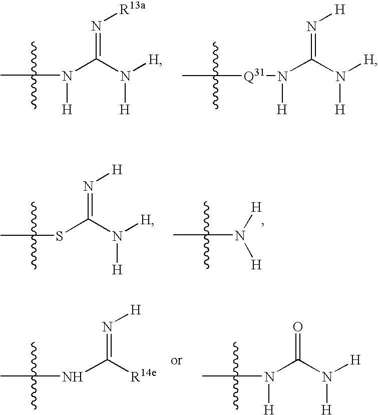
wherein R^{13a} is as defined above, but preferably represents OH, CN or NH₂ and Q³¹ and R^{14e} are as defined above.

More preferred definitions of R^{a2} and R^{a3} include —N(H)R^{14c}, wherein R^{14c} represents C₁₋₂ alkyl or, preferably, H.

Compounds of formula I that are more preferred still include those in which the group G-L takes any of the following definitions. 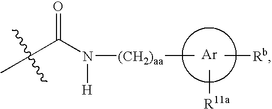
wherein aa represents 0, 1 or 2 (such as 2 or, particularly, 1);
R^{b} is as hereinbefore defined, but particularly represents tetrazol-1-yl, H, 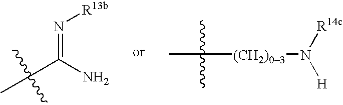
(e.g. one of the latter three groups),
wherein R^{13b} is as hereinbefore defined, but particularly represents NH₂ or, preferably, H;
R^{14c} is as hereinbefore defined, but particularly represents C₁₋₂ alkyl optionally substituted by one to 3 F atoms (e.g. CH₂CF₃), H, cyclopentyl or C(O)O—C₃₋₄ alkyl (e.g. one of the latter three groups);
R^{11a} is as hereinbefore defined, but,
(i) when R^{b} represents H, R^{11a} particularly represents one to three substituents selected from F, Cl, OH, methyl (which latter group is optionally substituted by OH or, particularly, C(O)N(R^{12b})R^{12c}) and methoxy (which latter group is substituted by C(O)N(H)R^{12b}),
(ii) when R^{b} represents —C(═NR^{13b})NH₂, R^{11a} particularly represents one or two substituents selected from F and OH or, preferably, R^{11a} represents H,
(iii) when R^{b} represents —(CH₂)₀₋₃—N(H)R^{14c}, R^{11a} particularly represents H or one or two substituents selected from F, Cl, OH, methyl, methoxy and CF₃ (e.g. H or one or two substituents selected from Cl, OH and methyl or, preferably, a single Cl substituent). 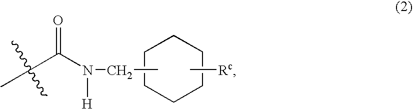
wherein R^{c} represents —C(═NR^{13b})NH₂ or, particularly, —N(H)R^{14c}, which groups are preferably attached in the 4-position relative to the point of attachment of the CH₂ group;
R^{13b} and R^{14c} are as hereinbefore defined, but preferably represent H. 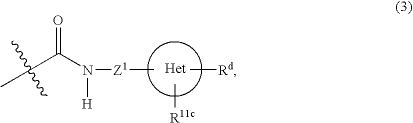
wherein Z¹ represents —CH₂C≡C—, —CH═CH—, C(O)CH₂ or, preferably, C(O) or —(CH₂)_{ab}—;
when Z¹ represents —CH₂C≡C—, —CH═CH—, Het represents a 5-membered, aromatic heterocyclic group containing one or, particularly, two nitrogen atoms;
when Z¹ represents C(O)CH₂, Het represents a 6-membered, fully saturated heterocyclic group containing one or, particularly, two nitrogen atoms;
when Z¹ represents C(O), Het represents a 6-membered, aromatic heterocyclic group containing two nitrogen atoms or, particularly, one nitrogen atom;
when Z¹ represents —(CH₂)_{ab}— Het represents a 5- or 6-membered monocyclic or 9- or 10-membered ring-fused bicyclic heterocyclic group containing, as heteroatom(s)
(a) a sulfur atom, or
(b) a nitrogen atom and, optionally, one or two further heteroatoms selected from nitrogen, oxygen and sulfur,
which heterocyclic group
(i) when 5- or 6-membered, is fully aromatic or fully saturated,
(ii) when 9- or 10-membered, is fully aromatic or part-aromatic;
ab represents 0 to 3, but preferably represents 1 or 2 or, when Het is 5-membered, also preferably represents 3;
R^{d} represents H, —C(═NR^{13b})NH₂ or —N(H)R^{14c}, but R^{d}, when Het is 5 or 10-membered, particularly represents —N(H)R^{14c};
R^{11c} is as hereinbefore defined, but preferably represents H or
(I) when Het is 6-membered and aromatic (e.g. a pyridinyl group), one or two substituents selected from F, Cl, methyl and CH₂OH,
(II) when Het is 6-membered and fully saturated, a methyl or a ═NH substituent;
R^{13b} is as hereinbefore defined, but preferably represents H;
R^{14c} is as hereinbefore defined, but preferably represents H or, when Het is 6-membered, methyl. 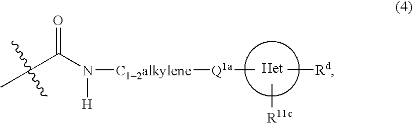
wherein Q^{1a} represents O or NR^{10a};
R^{10a} represents H, methyl or —CH₂CH₂OH;
Het represents a 6-membered or 10-membered, aromatic heterocyclic group containing two nitrogen atoms or, preferably, one nitrogen atom;
R^{d} represents H or —N(H)R^{14c};
R^{14c} is as hereinbefore defined, but preferably represents H;
R^{11c} is as hereinbefore defined, but preferably represents H or, when Het contains two nitrogen atoms, represents Cl. 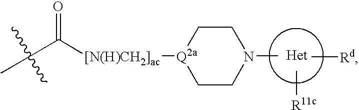
wherein Q^{2a} represents N or CH;
ac represents 0 or 1, but, when Q^{2a} represents CH, preferably represents 1;
Het represents a 6-membered, aromatic heterocyclic group containing two nitrogen atoms or, preferably, one nitrogen atom (e.g. a pyridinyl group, such as a pyridin-4-yl group);
R^{d} and R^{11c} are as hereinbefore defined, but preferably represent H; 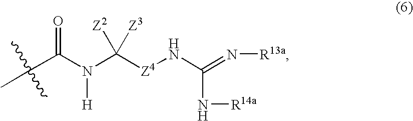
wherein Z² and Z³ independently represent H or F, but, preferably, Z² and Z³ both represent H or both represent F;
Z⁴ represents —(CH₂)₂C(O)— or, preferably, —CH₂C(O)—, —CH₂O—, —CH₂—C(H)═N— or —C(H)═N—;
R^{13a} and R^{14a} are as hereinbefore defined, but preferably represent H.

Particularly preferred compounds of the invention are compounds of formula Ia 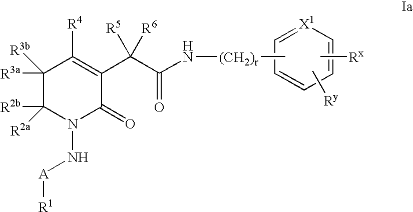
wherein X¹ represents CH or N;
when X¹ represents CH
(a) R^{x} takes the same definitions as R^{b} above, and
(b) R^{y} takes the same definitions as R^{11a} above;
   when X¹ represents N
(a) R^{x} takes the same definitions as R^{d} above, and
(b) R¹ takes the same definitions as R^{11c} above;
   r represents 1 to 3; and
   R¹, R^{2a}, R^{2b}, R^{3a}, R^{3b}, R⁴, R⁵, R⁶, R^{11a}, R^{11c}, R^{b}, R^{d} and A are as defined above,
   which compounds are also referred to hereinafter as “the compounds of the invention”.

Preferred compounds of formula Ia include those in which:
when X¹ represents CH, R^{x} represents tetrazol-1-yl, H, (CH₂)₁₋₂N(H)R^{14c} (e.g. CH₂N(H)R^{14c}) or 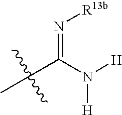
(e.g. any one of the latter three groups);
when X¹ represents N, R^{x} represents H or —N(H)R^{14c};
when X¹ represents CH, R¹ represents H or one to three substituents selected from halo, C₁₋₂ alkyl, C₁₋₂ alkoxy (which latter two groups are optionally substituted by one or more F atoms), OH, CH₂OH and OCH₂C(O)N(H)R^{12b} (e.g. H or one to three halo atoms);
when X¹ represents N, R^{y} represents H or one to three substituents selected from halo and C₁₋₂ alkyl;
R^{12b} represents H or, preferably, C₁₋₃ alkyl optionally substituted by N(CH₃)₂ (e.g. ethyl or (CH₂)₂₋₃N(CH₃)₂, particularly (CH₂)₃N(CH₃)₂);
r represents 2 or, particularly, 1.

Particularly preferred compounds of formula Ia include those in which:

A represents C(O), S(O)₂, C(O)NH (in which latter group the NH moiety is attached to R¹) or C₁₋₂ alkylene (which latter group is optionally substituted by one or more F atoms, but is preferably unsubstituted);

R¹ represents

(a) C₁₋₃ alkyl substituted by phenyl (which latter group is optionally substituted by one or more substituents selected from halo, C₁₋₄ alkyl and C₁₋₄ alkoxy (which latter two groups are optionally substituted by one or more F atoms)),
(b) phenyl or naphthyl (which latter two groups are optionally substituted by one or more substituents selected from CN, halo, C₁₋₄ alkyl and C₁₋₄ alkoxy (which latter two groups are optionally substituted by one or more F atoms) (e.g. one or more substituents selected from halo, C₁₋₄ alkyl and C₁₋₄ alkoxy (which latter two groups are optionally substituted by one or more F atoms)),
(c) a 5- or 6-membered monocyclic (preferably aromatic) heterocyclic group containing, as heteroatom(s), an oxygen or sulfur atom and/or one to three nitrogen atoms, which heterocyclic group is optionally substituted by one to four substituents selected from F, Cl, Br, ═O, OH, C₁₋₄ alkyl, C₁₋₄ alkoxy and Het^{c} (e.g. one to four substituents selected from F, Cl, Br and C₁₋₄ alkyl), or
(d) a 9- or 10-membered bicyclic (preferably part-aromatic) heterocyclic group containing one to three heteroatoms selected from oxygen, nitrogen and/or sulfur (e.g. two oxygen atoms), which heterocyclic group is optionally substituted by one to four substituents selected from F, Cl, Br, C₁₋₄ alkyl and C₁₋₄ alkoxy
(e.g. R¹ represents a group as defined at (a) to (c) above);
   Het^{c} represents a 5- or 6-membered monocyclic aromatic heterocyclic group containing, as heteroatom(s), an oxygen or sulfur atom and/or or two nitrogen atoms, which heterocyclic group is optionally substituted by one to four substituents selected from F, Cl, Br, C₁₋₄ alkyl and C₁₋₄ alkoxy;
   R^{2a}, R^{2b}, R^{3a}, R^{3b} all represent H;
   R⁴ represents H, methyl or halo (such as Cl);
   R⁵ and R⁶ both represent H;
   when X¹ represents CH and R^{x} represents H, then R^{y} represents one to three substituents selected from OH, methyl, CH₂OH, OCH₂C(O)N(H)R^{12b} and halo (particularly one to three halo atoms (e.g. one to three Cl atoms, such as two Cl atoms attached in the 2- and 5-positions relative to the point of attachment of the (CH₂)ᵣ group));
   when X¹ represents CH and R^{x} represents (CH₂)₁₋₂N(H)R^{14c}, then R^{y} represents H or, preferably, one or two substituents selected from halo, C₁₋₂ alkyl and C₁₋₂ alkoxy (which latter two groups are optionally substituted by one or more F atoms) (and particularly R^{y} represents one or two halo atoms (e.g. one or two Cl atoms, such as a Cl atom attached in the 3-position relative to the point of attachment of the (CH₂)ᵣ group));
   when X¹ represents CH and R^{x} represents tetrazol-1-yl, then R^{y} represents one or two halo (e.g. Cl atoms) or, preferably, H;
   when X¹ represents CH and R^{x} represents 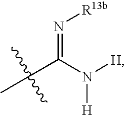
   then R^{y} represents one or two F atoms or, preferably, H;
   when X¹ represents CH, the group 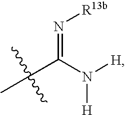
   if present, is attached at the 3- or, preferably, the 4-position relative to the point of attachment of the (CH₂)ᵣ group;
   when X¹ represents CH, the group (CH₂)₁₋₂N(H)R^{14c}, if present, is attached at the 5- or, preferably, the 6-position relative to the point of attachment of the (CH₂)ᵣ group;
   when X¹ represents CH, the tetrazol-1-yl group, if present, is attached at the 5- or, preferably, the 6-position relative to the point of attachment of the (CH₂)ᵣ group;
   R^{13b} represents OH, OCH₃ or preferably, C(O)OCH₂-phenyl or H (e.g. OH, C(O)OCH₂-phenyl or H);
   when X¹ represents N and R^{x} represents H, R^{y} represents H or, preferably, one or two substituents selected from halo (e.g. F) and methyl;
   when X¹ represents N and R^{x} represents —N(H)R^{14c}, R^{y} represents H or one or two methyl groups (e.g. H or methyl);
   R^{14c} represents CH₂CF₃, H, cyclopentyl or C(O)O—C₄ alkyl (e.g. one of the latter three groups, such as C(O)O—C₄ alkyl (e.g. C(O)O-tert-butyl) or, preferably, H).

Compounds of formula Ia that are more preferred still include those in which:

A represents C(O), C(O)NH (in which latter group the NH moiety is attached to R¹) or, particularly, S(O)₂ or C₁₋₂ alkylene (which latter group is optionally gem-disubstituted by two F atoms, but is preferably unsubstituted);

R¹ represents

(a) C₁₋₂ alkyl substituted by phenyl (which latter group is optionally substituted by one or more substituents selected from F, Cl and Br),
(b) phenyl (which latter group is optionally substituted by one or more substituents selected from F, Cl, Br, C₁₋₃ alkyl and C₁₋₃ alkoxy (which latter two groups are optionally substituted by one or more F atoms, but are preferably unsubstituted)),
(c) naphthyl (e.g. 1-naphthyl),
(d) pyridinyl (e.g. pyridin-2-yl or pyridin-3-yl) optionally substituted by one or two substituents selected from F, Cl, OH, C₁₋₄ alkyl (e.g. methyl) or, particularly, C₁₋₄ alkoxy (e.g. tert-butoxy or methoxy),
(e) pyridonyl (e.g. 2-pyridon-3-yl) optionally substituted by one or two substituents selected from F, Cl, and C₁₋₄ alkyl (e.g. methyl);
(f) a 5-membered aromatic heterocyclic group containing, as heteroatom(s), an oxygen or sulfur atom and/or one or two nitrogen atoms (e.g. pyrazolyl or thienyl), which heterocyclic group is optionally substituted by one to four (e.g. one to three) substituents selected from F, Cl, C₁₋₄ alkyl (e.g. methyl), C₁₋₄ alkoxy (e.g. methoxy) and pyridinyl (e.g. pyridin-2-yl) or
(f) quinolinyl, benzomorpholinyl, benzodioxanyl, benzo[c]oxa-1,2,5-diazolyl, 2,3-dihydrobenzofuranyl or, particularly, benzodioxolyl, all of which groups are optionally substituted by one or more (e.g. one to three) substituents selected from F, Cl, C₁₋₂ alkyl and C₁₋₂ alkoxy
(e.g. R¹ represents a group as defined at (a) to (c) above);
   R⁴ represents methyl;
   X¹ represents CH or N (e.g. CH);
   R^{x} represents (e.g. when X¹ represents CH) 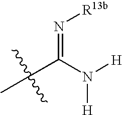
   attached at the 4-position relative to the point of attachment of the (CH₂)ᵣ group, or R^{x} may also represent tetrazol-1-yl or, particularly, CH₂N(H)R^{14c} (which latter two groups are attached, for example, in the 6-position relative to the point of attachment of the (CH₂)ᵣ group);
   R^{x} may alternatively represent H when X¹ represents CH and R^{y} represents one to three substituents selected from OH, methyl, CH₂OH, OCH₂C(O)N(H)R^{12b} and halo;
   R^{13b} represents C(O)OCH₂-phenyl or, preferably, H;
   R^{14c} represents C(O)O-tert-butyl or, particularly, H, ethyl, CH₂CF₃ or cyclopentyl (e.g. H or cyclopentyl).

In one embodiment of compounds of formula Ia that are more preferred still, R^{x} represents 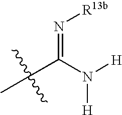
attached at the 4-position relative to the point of attachment of the (CH₂)ᵣ group.

Other preferred compounds of formula Ia include those in which:

A represents CH₂, (CH₂)₂ or CF₂CH₂ (in which latter group the CF₂ unit is attached to R¹);

R¹ represents

(a) phenyl optionally substituted by one or two substituents selected from halo (e.g. F or Cl), methyl, CF₃ and methoxy,
(b) pyrazolyl (e.g. pyrazol-4-yl) optionally substituted by one to three substituents selected from Cl and methyl,
(c) thienyl (e.g. thien-2-yl) optionally substituted by Cl or pyridinyl (e.g. pyridin-2-yl),
(d) pyridinyl (e.g. pyridin-2yl or pyridin-3-yl) optionally substituted by OH or methoxy,
(e) pyridonyl (e.g. 2-pyridon-3-yl) or
(f) benzodioxolyl (e.g. 5-benzodioxolyl) optionally substituted by halo (e.g. Cl);
   the group 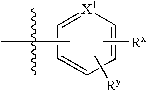
   represents 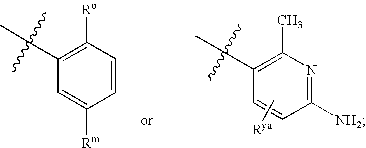
   R^{o} represents H, F, Cl, OH, methyl or, particularly, tetrazol-1-yl, OCH₂C(O)N(H)R^{12b} or CH₂N(H)R^{14c};
   R^{m} represents H, methyl, CF₃, methoxy, F or, particularly, Cl (for example:
(a) when R^{o} represents H or Cl, then R^{m} represents Cl;
(b) when R^{o} represents OH or methyl, then R^{m} represents F or, particularly Cl; and
(c) when R^{o} represents tetrazol-1-yl, OCH₂C(O)N(H)R^{12b} or CH₂N(H)R^{14c} then R^{m} represents H, methyl, CF₃, methoxy, F or, most preferably, Cl);
   R^{ya} represents H or, particularly, methyl.

Particularly preferred compounds of the invention are also compounds of formula Ib and Ic 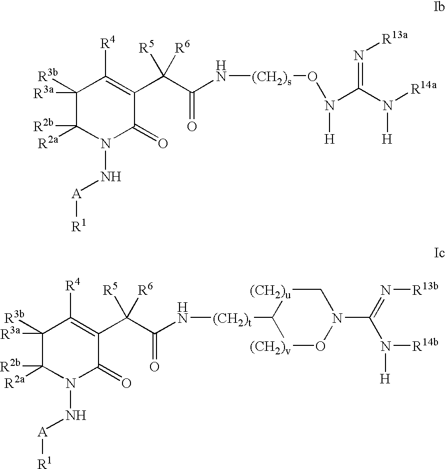
wherein s represents 2 to 4;
t represents 1 to 3;
u and v independently represent 0 to 2, the sum of u and v being 1 or 2; and
R¹, R^{2a}, R^{2b}, R^{3a}, R^{3b}, R⁴, R⁵, R⁶, R^{13a}, R^{13b}, R^{14a}, R^{14b} and A are as defined above,
which compounds are also referred to hereinafter as “the compounds of the invention”.

Preferred compounds of formula Ib include those in which:

s represents 3 or, particularly, 2;

R^{13a} and R^{14a} both represent H.

Preferred compounds of formula Ic include those in which:

t represents 2 or, particularly, 1;

u and v both represent 1;

R^{13b} and R^{14b} both represent H.

For the avoidance of doubt, the preferred definitions of groups given above in relation to compounds of formula Ia are also, where relevant, preferred definitions of the equivalent groups in compounds of formulae I, Ib and Ic. Moreover, references herein to compounds of formula I also include, where relevant, references to compounds of formula Ia, formula Ib and/or formula Ic.

Preferred compounds of the invention include the compounds of the Examples disclosed hereinafter.

Preparation

Compounds of formula I (including compounds of formulae Ia, Ib and Ic) may be made in accordance with techniques well known to those skilled in the art, for example as described hereinafter.

According to a further aspect of the invention there is provided a process for the preparation of a compound of formula I, which comprises:
(a) for compounds of formula I in which the group G represents
   (i) C(O)N(R^{8a})—[CH(C(O)R⁹)]₀₋₁—C₀₋₃ alkylene-(Q¹)ₐ-,
   (ii) C(O)N(R^{8b})—C₂₋₃ alkenylene-(Q¹)ₐ-,
   (iii) C(O)N(R^{8b})—C₂₋₃ alkynylene-(Q¹)ₐ-, 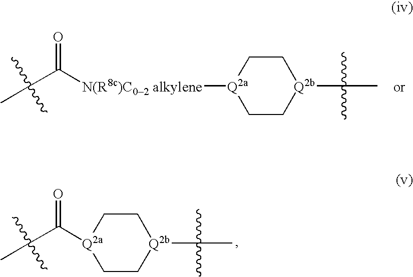
      wherein Q^{2a} represents N or NHCH,
         coupling of a compound of formula II, 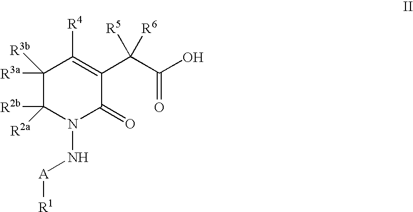
         wherein R¹, R^{2a}, R^{2b}, R^{3a}, R^{3b}, R⁴, R⁵, R⁶ and A are as hereinbefore defined, with a compound of formula III,
         H-G^{a}-L III
         wherein L is as hereinbefore defined and G^{a} represents
   (i) —N(R^{8a})—[CH(C(O)R⁹)]₀₋₁—C₀₋₃ alkylene-(Q¹)ₐ-,
   (ii) —N(R^{8b})—C₂₋₃ alkenylene-(Q¹)ₐ-,
   (iii) —N(R^{8b})—C₂₋₃ alkynylene-(Q¹)ₐ-, 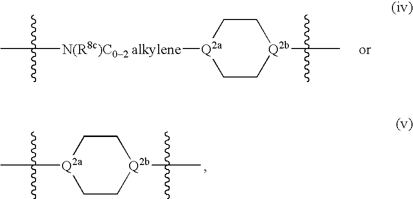
      wherein Q², represents N or NHCH and R^{8a}, R^{8b}, R^{8c}, R⁹, Q¹, Q^{2b} and a are as hereinbefore defined,
         for example in the presence of a coupling agent (e.g. oxalyl chloride in DMF, EDC, DCC, HBTU, HATU, PyBOP or TBTU), an appropriate base (e.g. pyridine, DMAP, TEA, 2,4,6-collidine or DIPEA) and a suitable organic solvent (e.g. dichloromethane, acetonitrile, EtOAc or DMF);
         (b) for compounds of formula I in which G represents 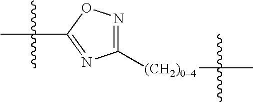
         and L represents L^{a}, which latter group represents L as hereinbefore defined, except that it does not represent C₀ alkylene-R^{a}, cyclisation of a compound of formula IV, 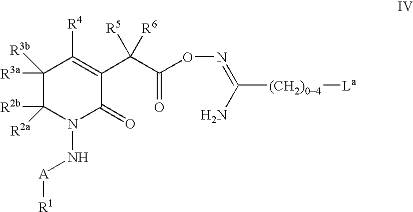
         wherein R¹, R^{2a}, R^{2b}, R^{3a}, R^{3b}, R⁴, R⁵, R⁶, L^{a} and A are as hereinbefore defined, for example at elevated temperature (e.g. 60° C. to reflux) in the presence of a suitable solvent (e.g. pyridine, toluene, 1,4-dioxane or THF) and optionally in the presence of a suitable catalyst (e.g. (n-Bu)₄NF, which may particularly be employed when the reaction solvent is THF);
         (c) for compounds of formula I in which R^{a}, R^{b}, R^{c} or R^{d} represents —C(═NH)NH₂, —C(═NHNH₂)NH₂ or —C(═NOH)NH₂, reaction of a compound of formula V, 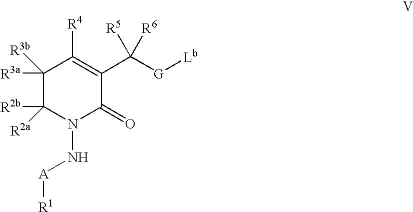
         wherein L^{b} represents L as hereinbefore defined, except that R^{a}, R^{b}, R^{c} or R^{d} (as appropriate) is replaced by a cyano or —C(═NH)O—C₁₋₄ alkyl group, and R¹, R^{2a}, R^{2b}, R^{3a}, R^{3b}, R⁴, R⁵, R⁶, G and A are as hereinbefore defined, with a suitable source of ammonia, hydrazine or hydroxylamine (e.g. ammonia gas, ammonium acetate, hydrazine, hydrazine monohydrochloride, hydroxylamine or hydroxylamine hydrochloride) under conditions known to those skilled in the art (e.g. conditions such as those described in Tetrahedron Lett. 40, 7067 (1999)), for example from ambient (e.g. 15 to 25° C.) to elevated temperature (e.g. 60° C. to reflux) in the presence of a suitable solvent (e.g. ethanol);
         (d) for compounds of formula I in which R^{13a}, R^{13b} or R^{13c} represents H, deprotection of a corresponding compound of formula I in which R^{13a}, R^{13b} or R^{13c} (as appropriate) represents C(O)O—CH₂aryl (e.g. C(O)O-benzyl), for example under conditions known to those skilled in the art (such as hydrogenation in the presence of an appropriate catalyst (e.g. Pt/C or, particularly, Pd/C), a suitable solvent (e.g. an alcohol such as ethanol or, particularly, methanol) and, optionally, an acid (e.g. HCl));
         (e) for compounds of formula I in which R^{14c} represents H, deprotection of a corresponding compound of formula I in which R^{14c} represents C(O)O—C₁₋₆ alkyl (e.g. C(O)O-tert-butyl), for example under conditions known to those skilled in the art (e.g. acid or base hydrolysis, such as, for deprotection of compounds in which R^{14c} represents C(O)O-tert-butyl, reaction with HCl gas in the presence of a suitable solvent (e.g. an alcohol such as ethanol or, particularly, methanol), or reaction with trifluoroacetic acid at sub-ambient temperature (e.g. 0 to 4° C.), optionally in the presence of a suitable solvent such as DCM);
         (f) reaction of a compound of formula VI, 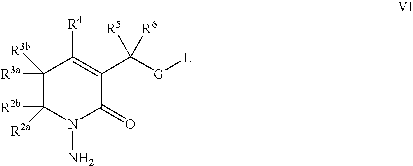
         wherein R^{2a}, R^{2b}, R^{3a}, R^{3b}, R⁴, R⁵, R⁶, G and L are as hereinbefore defined, with a compound of formula VII,
         R¹-A-Lg² VII
         wherein Lg² represents a suitable leaving group (e.g. halo, trifluoromethanesulfonate or OH) and R¹ and A are as hereinbefore defined, for example under conditions known to those skilled in the art (such as at sub-ambient temperature (e.g. 0° C.) in the presence of an appropriate base (e.g. K₂CO₃ or pyridine) and a suitable solvent (e.g. DCM));
         (g) for compounds of formula I in which A represents C(O)NH, reaction of a compound of formula VI, as hereinbefore defined, with a compound of formula VIII,
         R¹—N═C═O VIII
         wherein R¹ is as hereinbefore defined, for example under conditions known to those skilled in the art (such as at ambient temperature (e.g. 15 to 25° C.) in the presence of a suitable solvent (e.g. DCM));
         (h) for compounds of formula I in which A represents C₁₋₆ alkylene, reaction of a compound of formula VI, as hereinbefore defined, with a compound of formula IX,
         R¹—C₀₋₅ allylene-CHO IX
         wherein R¹ is as hereinbefore defined, for example under conditions known to those skilled in the art (such as at reflux in the presence of a suitable solvent (e.g. ethanol), followed by reduction in the presence of a reducing agent (e.g. NaBH₃CN), for example under conditions known to those skilled in the art (e.g. at ambient temperature (such as 15 to 25° C.) in the presence of a suitable solvent (such as ethanol); or
         (i) for compounds of formula I in which R^{a}, R^{b}, R^{c} or R^{d} represents —C(═NCN)NH₂, reaction of a corresponding compound of formula I in which R^{a}, R^{b}, R^{c} or R^{d}, respectively, represents —C(═NH)NH₂ with cyanogen bromide, for example under conditions known to those skilled in the art (e.g. in the presence of a suitable base (such as an alkali metal alkoxide like sodium ethoxide) and an appropriate solvent (such as a lower alkyl alcohol like ethanol).

Compounds of formula II may be prepared by hydrolysis of a compound of formula X, 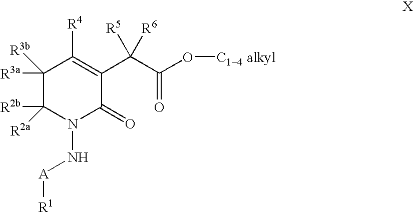
wherein R¹, R^{2a}, R^{2b}, R^{3a}, R^{3b}, R⁴, R⁵, R⁶ and A are as hereinbefore defined, for example under conditions known to those skilled in the art (e.g. by basic hydrolysis in the presence of an alkali metal hydroxide (e.g. NaOH or, particularly, LiOH) and a suitable solvent (e.g. water, THF or a mixture thereof)).

Compounds of formula IV may be prepared by the coupling of a compound of formula II, as hereinbefore defined, with a compound of formula XI, 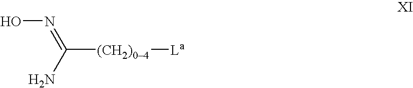
wherein L^{a} is as hereinbefore defined, for example under conditions well know to those skilled in the art (e.g. those described in WO 01/19262, such as at ambient temperature (e.g. 15 to 25° C.) in the presence of a coupling agent (e.g. EDC) and a suitable solvent (e.g. DMF)).

As the skilled person will appreciate, in some instances, compounds of formula V are identical to certain compounds of formula I (e.g. compounds in which R^{b}, R^{c} or R^{d} represents H and R^{11a}, R^{11b} or R^{11c}, respectively, represents CN). In this respect, compounds of formula V may be prepared by analogy with the procedures described herein for the preparation of compounds of formula I.

Compounds of formula VI may be prepared by reduction of a compound of formula XII, 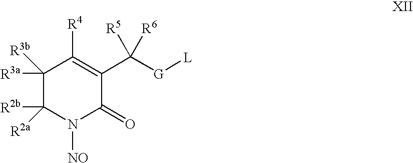
wherein R^{2a}, R^{2b}, R^{3a}, R^{3b}, R⁴, R⁵, R⁶, G and L are as hereinbefore defined, for example under conditions that are well known to those skilled in the art (such as by reaction with zinc metal (e.g. zinc powder or iron metal powder) in the presence of an appropriate acid (e.g. acetic acid or hydrochloric acid) and optionally in the presence of a suitable solvent (e.g. methanol)).

Compounds of formula IX may be prepared by oxidation of an alcohol of formula XIII,
R¹—C₀₋₅ alkylene-CH₂OH XIII
wherein R¹ is as hereinbefore defined, for example under conditions known to those skilled in the art, such as reaction with PCC, oxalyl chloride and DMSO (Swern oxidation) or, particularly, Dess-Martin periodinane in the presence of a suitable solvent (such as DCM).

Compounds of formula X may be prepared by reaction of a compound of formula XIV, 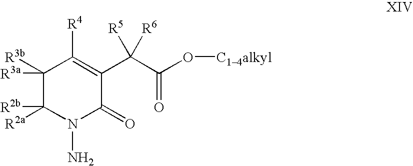
wherein R^{2a}, R^{2b}, R^{3a}, R^{3b}, R⁴, R⁵ and R⁶ are as hereinbefore defined, with a compound of formula VII, of formula VIII, or of formula IX, as hereinbefore defined, for example under conditions known to those skilled in the art (e.g. conditions described at process steps (f), (g) and (h) above in respect of compounds of formula I).

Compounds of formula XI may be prepared by methods well known to those skilled in the art. For example, compounds of formula XI may be prepared by reaction of a compound of formula XV or XVI, 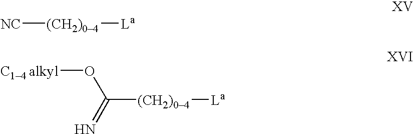
wherein L^{a} is as hereinbefore defined, with hydroxylamine or an acid addition salt thereof, for example under conditions described at process step (d) above in respect of compounds of formula I.

Compounds of formula XII may be prepared by analogy with compounds of formulae I and XVIII.

Compounds of formula XIII may be prepared by reduction of a carboxylic acid of formula XVII,
R¹—C₀₋₅ alkylene-C(O)OH XVII
wherein R¹ is as hereinbefore defined, for example under conditions known to those skilled in the art, such as reaction with LiAlH₄ or, particularly, borane in the presence of a suitable solvent (such as THF).

Compounds of formula XIV may be prepared by reduction of a compound of formula XVIII, 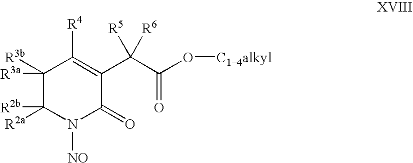
wherein R^{2a}, R^{2b}, R^{3a}, R^{3b}, R⁴, R⁵ and R⁶ are as hereinbefore defined, for example under conditions described hereinbefore in respect of the preparation of compounds of formula VI.

Compounds of formula XVIII may be prepared by nitrosation of a corresponding compound of formula XIX, 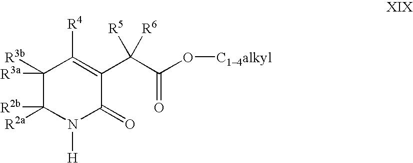
wherein R^{2a}, R^{2b}, R^{3a}, R^{3b}, R⁴, R⁵ and R⁶ are as hereinbefore defined, for example under conditions well known to those skilled in the art, e.g. reaction at with a nitrosating agent (such as nitrous acid, NOCl, N₂O₃, N₂O₄ or, particularly, a C₁₋₆ alkyl nitrite (e.g. tert-butyl nitrite)) in the presence of a suitable solvent (e.g. diethyl ether) and optionally in the presence of an appropriate base (e.g. pyridine).

Compounds of formula XIX may be prepared by α,β-elimination (relative to the oxo group of the piperidinone ring) of H-Lg³ from a piperidinone of formula XX, 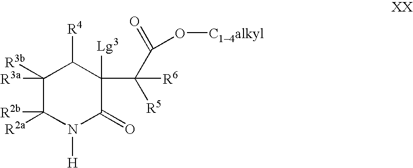
or a protected derivative thereof, wherein Lg³ represents a leaving group capable of undergoing thermal 1,2-elimination (e.g. —Se(O)-phenyl) and R^{2a}, R^{2b}, R^{3a}, R^{3b}, R⁴, R⁵ and R⁶ are as hereinbefore defined, for example under conditions that are well known to those skilled in the art (e.g. when Lg³ represents —Se(O)-phenyl, thermal elimination of Ph—Se—OH at ambient temperature (such as 15 to 25° C.) in the presence of a suitable solvent (such as DCM, water or a mixture thereof)).

Compounds of formula XX in which Lg³ represents —Se(O)-phenyl may be prepared by oxidation of a compound of formula XX, 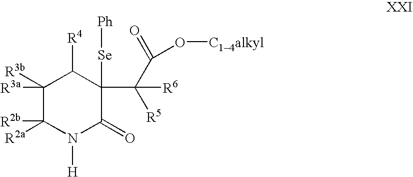
or a protected derivative thereof, wherein R^{2a}, R^{2b}, R^{3a}, R^{3b}, R⁴, R⁵ and R⁶ are as hereinbefore defined, for example under conditions well know to those skilled in the art (e.g. reaction at sub-ambient temperature (such as 0° C.) with an appropriate oxidising agent (such as mCPBA or, particularly, hydrogen peroxide) in the presence of a suitable solvent (such as DCM, water or a mixture thereof)).

As the skilled person will appreciate, the conversion of compounds of formula XXI to corresponding compounds of formula XIX may conveniently take place in a “one-pot” procedure, where the oxidised intermediate (the compound of formula XX in which Lg³ represents —Se(O)-phenyl) is not isolated and thermal elimination of Ph—Se—OH takes place during the “work-up” of the oxidation reaction.

Compounds of formula XX may be prepared by reaction of a compound of formula XXI, 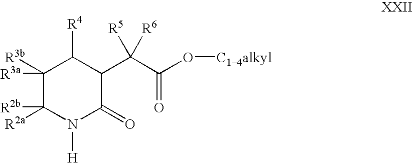
or a protected derivative thereof, wherein R^{2a}, R^{2b}, R^{3a}, R^{3b}, R⁴, R⁵ and R⁶ are as hereinbefore defined, with a compound of formula XXIII,
Phenyl-Se-Lg⁴ XXIII
wherein Lg⁴ represents a suitable leaving group (e.g. halo, such as Br, or —SePh), in the presence of an appropriate base (e.g. a metal hydride or, particularly, a metal amide (such as lithium bis(trimethylsilyl)amide)), for example under conditions known to those skilled in the art (e.g. at low temperature (such as −78° C.)) in the presence of a suitable solvent (such as THF).

Compounds of formula XXI may be prepared by reaction of a compound of formula XXIV, 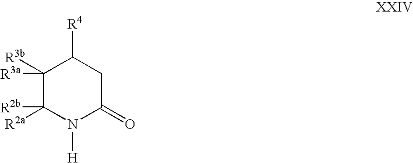
or a protected derivative thereof, wherein R^{2a}, R^{2b}, R^{3a}, R^{3b} and R⁴ are as hereinbefore defined, with a compound of formula XXV, 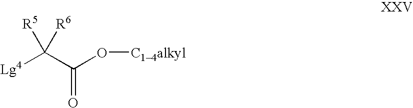
wherein Lg⁴, R⁵ and R⁶ are as hereinbefore defined, in the presence of an appropriate base (e.g. a metal hydride or, particularly, a metal amide (such as lithium bis(trimethylsilyl)amide)), for example under conditions known to those skilled in the art (e.g. at low temperature (such as −78 to −10° C.)) in the presence of a suitable solvent (such as THF).

Compounds of formula XXIV may be prepared by oxidation of a compound of formula XXVI, 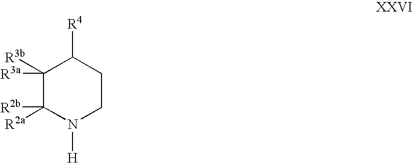
or a protected derivative thereof, wherein R^{2a}, R^{2b}, R^{3a}, R^{3b} and R⁴ are as hereinbefore defined, with a suitable oxidising agent (e.g. H₂O₂, (PhIO)ₙ, Hg(OAc)₂ or, particularly, RuO₄, which latter reagent may be formed in situ by oxidation of RuO₂ (e.g. by an excess of NaIO₄)), for example under conditions known to those skilled in the art (e.g. at ambient temperature (such as 15 to 25° C.) in the presence of a suitable solvent (such as ethyl acetate, water or a mixture thereof)).

As the skilled person will appreciate, the conversion of compounds of formula XXVI to corresponding compounds of formula XIX may require, at any or all of the reaction steps, protection of the N—H group of the piperidone ring system. Suitable protective groups for this purpose include benzyloxycarbonyl and, particularly, tert-butyloxycarbonyl. The protective group may be introduced and removed under conditions that are well known to those skilled in the art. The protective group may be conveniently introduced before the compound of formula XXVI is converted to the compound of XXV (e.g. by reaction, under conditions that are well known to those skilled in the art, of a compound of XXVI with di-tert-butyldicarbonate). Further, the protective group may be conveniently removed, again under conditions that are well known to those skilled in the art (e.g. by reaction with trifluoroacetic acid), once the compound of formula XIX has been formed.

Compounds of formulae III, VII, VIII, XV, XVI, XVII, XXIII and XXV are either commercially available, are known in the literature, or may be obtained by analogy with the processes described herein, or by conventional synthetic procedures, in accordance with standard techniques, from readily available starting materials using appropriate reagents and reaction conditions. In this respect, compounds described herein may also be obtained by analogy with synthetic procedures described in the prior art documents mentioned above (and WO 94/20467, WO 94/29336, WO 95/23609, WO 96/06832, WO 96/06849, WO 97/11693, WO 97/24135, WO 98/01422, WO 01/68605, WO 99/26920, WO 01/79155, WO 01/68605, WO 96/18644, WO 97/01338, WO 97/30708, WO 98/16547, WO 99/26926, WO 00/73302, WO 01/04117, WO 01/79262, WO 02/057225, WO 02/064140, WO 03/29224, U.S. Pat. No. 5,668,289, U.S. Pat. No. 5,792,779 and WO 95/35313 in particular).

Substituents on alkyl, alkenyl, cycloalkyl, cycloalkenyl, aryl and heterocyclic groups in compounds of formulae I, II, IV, V, VI, X, XII, XIV, XVIII, XIX, XX, XXI, XXII, XXIV and XXVI may be introduced and/or interconverted using techniques well known to those skilled in the art by way of standard functional groups interconversions, in accordance with standard techniques, from readily available starting materials using appropriate reagents and reaction conditions. For example, hydroxy may be converted to alkoxy, phenyl may be halogenated to give halophenyl, halo may be displaced by cyano, etc.

The skilled person will also appreciate that various standard substituent or functional group interconversions and transformations within certain compounds of formula I will provide other compounds of formula I. For example, hydroxyamidino may be reduced to amidino.

Compounds of formula I may be isolated from their reaction mixtures using conventional techniques.

In accordance with the present invention, pharmaceutically acceptable derivatives of compounds of formula I also include “protected” derivatives, and/or compounds that act as prodrugs, of compounds of formula I.

Compounds that may act as prodrugs of compounds of formula I that may be mentioned include compounds of formula I in which R^{13a}, R^{13b} or R^{13c} is other than H or R^{14c} represents C(O)O—C₁₋₆ alkyl, the alkyl part of which group is optionally substituted by aryl and/or one or more halo atoms (e.g. compounds in which R^{14c} represents C(O)O-tert-butyl).

The compounds of the invention may exhibit tautomerism. All tautomeric forms and mixtures thereof are included within the scope of the invention. Particular tautomeric forms that may be mentioned include those connected with the position of the double bond in the amidine or guanidine functionalities that the groups R^{a} to R^{d} may represent.

Compounds of the invention may also contain one or more asymmetric carbon atoms and may therefore exhibit optical and/or diastereoisomerism. Diastereoisomers may be separated using conventional techniques, e.g. chromatography. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the invention.

It will be appreciated by those skilled in the art that in the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkylsilyl or diarylalkylsilyl groups (e.g. t-butyldimethylsilyl, t-butyldiphenylsilyl or trimethylsilyl) and tetrahydropyranyl. Suitable protecting groups for carboxylic acid include C₁₋₆ alkyl or benzyl esters. Suitable protecting groups for amino and amidino include t-butyloxycarbonyl, benzyloxycarbonyl or 2-trimethylsilylethoxycarbonyl (Teoc). Amidino nitrogens may also be protected by hydroxy or alkoxy groups, and may be either mono- or diprotected.

The protection and deprotection of functional groups may take place before or after coupling, or before or after any other reaction in the above-mentioned schemes.

Protecting groups may be removed in accordance with techniques that are well known to those skilled in the art and as described hereinafter.

Persons skilled in the art will appreciate that, in order to obtain compounds of the invention in an alternative, and, on some occasions, more convenient, manner, the individual process steps mentioned hereinbefore may be performed in a different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

Protected derivatives of compounds of the invention may be converted chemically to compounds of the invention using standard deprotection techniques (e.g. hydrogenation). The skilled person will also appreciate that certain compounds of formula I (e.g. compounds in which R^{13a}, R^{13b} or R^{13c} is other than H) may also be referred to as being “protected derivatives” of other compounds of formula I (e.g. those in which R^{13a}, R^{13b} or R^{13c} represents H).

Those skilled in the art will also appreciate that certain compounds of formula I will be useful as intermediates in the synthesis of certain other compounds of formula I.

Some of the intermediates referred to hereinbefore are novel. According to a further aspect of the invention there is thus provided: (a) a compound of formula II, or a protected derivative thereof; (b) a compound of formula IV, or a protected derivative thereof; (c) a compound of formula V, or a protected derivative thereof; and (d) a compound of formula VI, or a protected derivative thereof.

Medical and Pharmaceutical Use

Compounds of the invention may possess pharmacological activity as such. However, other compounds of the invention (including compounds of formula I in which R^{13a}, R^{13b} or R^{13c} is other than H or R^{14c} represents C(O)O-tert-butyl) may not possess such activity, but may be administered parenterally or orally, and may thereafter be metabolised in the body to form compounds that are pharmacologically active (including, but not limited to, corresponding compounds of formula I in which R^{13a}, R^{13b}, R^{13c} or R^{14c} represents H). Such compounds (which also includes compounds that may possess some pharmacological activity, but that activity is appreciably lower than that of the “active” compounds to which they are metabolised), may therefore be described as “prodrugs” of the active compounds.

Thus, the compounds of the invention are useful because they possess pharmacological activity, and/or are metabolised in the body following oral or parenteral administration to form compounds which possess pharmacological activity. The compounds of the invention are therefore indicated as pharmaceuticals.

According to a further aspect of the invention there is thus provided the compounds of the invention for use as pharmaceuticals.

In particular, compounds of the invention are potent inhibitors of thrombin either as such and/or (e.g. in the case of prodrugs), are metabolised following administration to form potent inhibitors of thrombin, for example as may be demonstrated in the tests described below.

By “prodrug of a thrombin inhibitor”, we include compounds that form a thrombin inhibitor, in an experimentally-detectable amount, and within a predetermined time (e.g. about 1 hour), following oral or parenteral administration (see, for example, Test E below) or, alternatively, following incubation in the presence of liver microsomes (see, for example, Test F below).

The compounds of the invention are thus expected to be useful in those conditions where inhibition of thrombin is beneficial (as determined by reference to a clinically relevant end-point, e.g. conditions, such as thrombo-embolisms, where inhibition of thrombin is required or desired, and/or conditions where anticoagulant therapy is indicated), including the following:

The treatment and/or prophylaxis of thrombosis and hypercoagulability in blood and/or tissues of animals including man. It is known that hypercoagulability may lead to thrombo-embolic diseases. Conditions associated with hypercoagulability and thrombo-embolic diseases are usually designated as thrombophilia conditions. These conditions include, but are not limited to, inherited or acquired activated protein C resistance, such as the factor V-mutation (factor V Leiden), inherited or acquired deficiencies in antithrombin III, protein C, protein S, heparin cofactor II, and conditions with increased plasma levels of the coagulation factors such as caused by the prothrombin G20210A mutation. Other conditions known to be associated with hypercoagulability and thrombo-embolic disease include circulating antiphospholipid antibodies (Lupus anticoagulant), homocysteinemi, heparin induced thrombocytopenia and defects in fibrinolysis, as well as coagulation syndromes (e.g. disseminated intravascular coagulation (DIC)) and vascular injury in general (e.g. due to trauma or surgery). Furthermore, low physical activity, low cardiac output or high age are known to increase the risk of thrombosis and hypercoagulability may be just one of several factors underlying the increased risk. These conditions include, but are not limited to, prolonged bed rest, prolonged air travelling, hospitalisation for an acute medical disorder such as cardiac insufficiency or respiratory insufficiency. Further conditions with increased risk of thrombosis with hypercoagulability as one component are pregnancy and hormone treatment (e.g. oestrogen).

The treatment of conditions where there is an undesirable excess of thrombin without signs of hypercoagulability, for example in neurodegenerative diseases such as Alzheimer's disease.

Particular disease states which may be mentioned include the therapeutic and/or prophylactic treatment of venous thrombosis (e.g. deep venous thrombosis, DVT) and pulmonary embolism, arterial thrombosis (e.g. in myocardial infarction, unstable angina, thrombosis-based stroke and peripheral arterial thrombosis), and systemic embolism usually from the atrium during atrial fibrillation (e.g. non-valvular or valvular atrial fibrillation) or from the left ventricle after transmural myocardial infarction, or caused by congestive heart failure; prophylaxis of re-occlusion (i.e. thrombosis) after thrombolysis, percutaneous trans-luminal angioplasty (PTA) and coronary bypass operations; the prevention of thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, chronic obstructive lung disease, septic shock, septicemia, inflammatory responses, which include, but are not limited to, edema, acute or chronic atherosclerosis such as coronary arterial disease and the formation of atherosclerotic plaques, cardiac insufficiency, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

Compounds of the invention that inhibit trypsin and/or thrombin may also be useful in the treatment of pancreatitis.

The compounds of the invention are thus indicated both in the therapeutic and/or prophylactic treatment of these conditions.

According to a further aspect of the present invention, there is provided a method of treatment of a condition where inhibition of thrombin is required which method comprises administration of a therapeutically effective amount of a compound of the invention to a person suffering from, or susceptible to, such a condition.

The compounds of the invention will normally be administered orally, intravenously, subcutaneously, buccally, rectally, dermally, nasally; tracheally, bronchially, by any other parenteral route or via inhalation, in the form of pharmaceutical preparations comprising compound of the invention either as a free base, or a pharmaceutically acceptable non-toxic organic or inorganic acid addition salt, in a pharmaceutically acceptable dosage form.

Preferred route of administration of compounds of the invention are oral.

Depending upon the disorder and patient to be treated and the route of administration, the compositions may be administered at varying doses.

The compounds of the invention may also be combined and/or co-administered with any antithrombotic agent(s) with a different mechanism of action, such as one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than thrombin (e.g. synthetic FXa, FVIIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X₁, P2Y₁, P2Y₁₂ [P2T]) antagonists; and inhibitors of carboxypeptidase U (CPU or TAFIa) and inhibitors of plasminogen activator inhibitor-1 (PAI-1).

The compounds of the invention may further be combined and/or co-administered with thrombolytics such as one or more of tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

According to a further aspect of the invention there is thus provided a pharmaceutical formulation including a compound of the invention, in admixture with a pharmaceutically acceptable adjuvant, diluent or carrier.

Suitable daily doses of the compounds of the invention in therapeutic treatment of humans are about 0.001-100 mg/kg body weight at peroral administration and 0.001-50 mg/kg body weight at parenteral administration.

For the avoidance of doubt, as used herein, the term “treatment” includes therapeutic and/or prophylactic treatment.

Compounds of the invention have the advantage that they may be more efficacious, be less toxic, be longer acting, have a broader range of activity, be more selective (e.g. for inhibiting thrombin over other serine proteases, in particular trypsin and those involved in haemostasis), be more potent, produce fewer side effects, be more easily absorbed, and/or have a better pharmacokinetic profile (e.g. higher oral bioavailability and/or lower clearance), than, and/or have other useful pharmacological, physical, or chemical, properties over, compounds known in the prior art.

Biological Tests

The following test procedures may be employed.

Test A

Determination of Thrombin Clotting Time (TT)

The inhibitor solution (25 μL) is incubated with plasma (25 μL) for three minutes. Human thrombin (T 6769; Sigma Chem. Co or Hematologic Technologies) in buffer solution, pH 7.4 (25 μL, 4.0 NIH units/mL), is then added and the clotting time measured in an automatic device (KC 10; Amelung).

The thrombin clotting time (TT) is expressed as absolute values (seconds) as well as the ratio of TT without inhibitor (TT₀) to TT with inhibitor (TTᵢ). The latter ratios (range 1-0) are plotted against the concentration of inhibitor (log transformed) and fitted to sigmoidal dose-response curves according to the equation
y=a/[1+(x/IC₅₀)^{s}]
where: a=maximum range, i.e. 1; s=slope of the dose-response curve; and IC₅₀=the concentration of inhibitor that doubles the clotting time. The calculations are processed on a PC using the software program GraFit Version 3, setting equation equal to: Start at 0, define end=1 (Erithacus Software, Robin Leatherbarrow, Imperial College of Science, London, UK).
Test B
Determination of Thrombin Inhibition with a Chromogenic, Robotic Assay

The thrombin inhibitor potency is measured with a chromogenic substrate method, in a Plato 3300 robotic microplate processor (Rosys AG, CH-8634 Hombrechtikon, Switzerland), using 96-well, half volume microtitre plates (Costar, Cambridge, Mass., USA; Cat No 3690). Stock solutions of test substance in DMSO (72 μL), 0.1-1 mmol/L, are diluted serially 1:3 (24+48 μL) with DMSO to obtain ten different concentrations, which are analysed as samples in the assay. 2 μL of test sample is diluted with 124 μL assay buffer, 12 μL of chromogenic substrate solution (S-2366, Chromogenix, Mölndal, Sweden) in assay buffer and finally 12 μL of α-thrombin solution (Human α-thrombin, Sigma Chemical Co. or Hematologic Technologies) in assay buffer, are added, and the samples mixed. The final assay concentrations are: test substance 0.00068-133 μmol/L, S-2366 0.30 mmol/L, α-thrombin 0.020 NIHU/mL. The linear absorbance increment during 40 minutes incubation at 37° C. is used for calculation of percentage inhibition for the test samples, as compared to blanks without inhibitor. The IC₅₀-robotic value, corresponding to the inhibitor concentration which causes 50% inhibition of the thrombin activity, is calculated from a log concentration vs. % inhibition curve.

Test C

Determination of the Inhibition Constant Kᵢ for Human Thrombin

Kᵢ-determinations are made using a chromogenic substrate method, performed at 37° C. on a Cobas Bio centrifugal analyser (Roche, Basel, Switzerland). Residual enzyme activity after incubation of human α-thrombin with various concentrations of test compound is determined at three different substrate concentrations, and is measured as the change in optical absorbance at 405 nm.

Test compound solutions (100 μL; normally in buffer or saline containing BSA 10 g/L) are mixed with 200 μL of human α-thrombin (Sigma Chemical Co) in assay buffer (0.05 mol/L Tris-HCl pH 7.4, ionic strength 0.15 adjusted with NaCl) containing BSA (10 g/L), and analysed as samples in the Cobas Bio. A 60 μL sample, together with 20 μL of water, is added to 320 μL of the substrate S-2238 (Chromogenix AB, Mölndal, Sweden) in assay buffer, and the absorbance change (ΔA/min) is monitored. The final concentrations of S-2238 are 16, 24 and 50 μmol/L and of thrombin 0.125 NIHU/mL.

The steady state reaction rate is used to construct Dixon plots, i.e. diagrams of inhibitor concentration vs. 1/(ΔA/min). For reversible, competitive inhibitors, the data points for the different substrate concentrations typically form straight lines which intercept at x=−Kᵢ.

Test D

Determination of Activated Partial Thromboplastin Time (APTT)

APTT is determined in pooled normal human citrated plasma with the reagent PTT Automated 5 manufactured by Stago. The inhibitors are added to the plasma (10 μL inhibitor solution to 90 μL plasma) and incubated with the APTT reagent for 3 minutes followed by the addition of 100 μL of calcium chloride solution (0.025 M) and APTT is determined by use of the coagulation analyser KC10 (Amelung) according to the instructions of the reagent producer.

The clotting time is expressed as absolute values (seconds) as well as the ratio of APTT without inhibitor (APTT₀) to APTT with inhibitor (APTTᵢ). The latter ratios (range 1-0) are plotted against the concentration of inhibitor (log transformed) and fitted to sigmoidal dose-response curves according to the equation
y=a/[1+(x/IC₅₀)^{s}]
where: a=maximum range, i.e. 1; s=slope of the dose-response curve; and IC₅₀=the concentration of inhibitor that doubles the clotting time. The calculations are processed on a PC using the software program GraFit Version 3, setting equation equal to: Start at 0, define end=1 (Erithacus Software, Robin Leatherbarrow, Imperial College of Science, London, UK). IC₅₀APTT is defined as the concentration of inhibitor in human plasma that doubled the Activated Partial Thromboplastin Time.
Test E
Determination of Plasma Clearance and Oral Bioavailability in Rat

Plasma clearance and oral bioavailability are estimated in female Sprague Dawley rats. The compound is dissolved in water or another appropriate vehicle. For determination of plasma clearance the compound is administered as a subcutaneous (sc) or an intravenous (iv) bolus injection at a dose of 1-4 μmol/kg. Blood samples are collected at frequent intervals up to 24 hours after drug administration. For bioavailability estimates, the compound is administered orally at 10 μmol/kg via gavage and blood samples are collected frequently up to 24 hours after dosing. The blood samples are collected in heparinized tubes and centrifuged within 30 minutes, in order to separate the plasma from the blood cells. The plasma is transferred to plastic vials with screw caps and stored at −20° C. until analysis. Prior to the analysis, the plasma is thawed and 50 μL of plasma samples are precipitated with 150 μL of cold acetonitrile. The samples are centrifuged for 20 minutes at 4000 rpm. 75 μL of the supernatant is diluted with 75 μL of 0.2% formic acid. 10 μL volumes of the resulting solutions are analysed by LC-MS/MS and the concentrations of thrombin inhibitor are determined using standard curves. All pharmacokinetic calculations are performed with the computer program WinNonlin™Professional (Pharsight Corporation, California, USA), or an equivalent program. Area under the plasma concentration-time profiles (AUC) is estimated using the log/linear trapezoidal rule and extrapolated to infinite time. Plasma clearance (CL) of the compound is then determined as
CL=Dose(iv/sc)/AUC(iv/sc).

The oral bioavailability is calculated as
F=CL×AUC(po)/Dose(po).

Plasma clearance is reported as mL/min/kg and oral bioavailability as percentage (%).

Test F

Determination of In Vitro Stability

Liver microsomes are prepared from Sprague-Dawley rats and human liver samples according to internal SOPs. The compounds are incubated at 37° C. at a total microsome protein concentration of 0.5 mg/mL in a 0.1 mol/L potassium phosphate buffer at pH 7.4, in the presence of the cofactor, NADPH (1.0 mmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding an equal volume of acetonitrile containing 0.8% formic acid. The concentration of compound remaining in each of the collected samples is determined by means of LC-MS/MS. The elimination rate constant (k) of the thrombin inhibitor is calculated as the slope of the plot of ln[Thrombin inhibitor] against incubation time (minutes). The elimination rate constant is then used to calculate the half-life (T_{1/2}) of the thrombin inhibitor, which is subsequently used to calculate the intrinsic clearance (CLint) of the thrombin inhibitor in liver microsomes as:

$$CLint ( in µl \text{/} min \text{/} mg ) = \frac{( ln 2 \times incubation volume )}{( {T}_{1 / 2} \times protein concentration )}$$

Test G
Venous Thrombosis Model

The thrombogenic stimuli are vessel damage and blood flow stasis. Rats are anaesthetised and the abdomen is opened. A partial occlusion on the caval vein, caudal to the left kidney-vein, is obtained with a snare around the vein and a cannula, which is than removed. A filter-paper soaked with FeCl₃ is placed on the external surface of the distal part of the caval vein. The abdomen is filled with saline and closed. At the end of the experiment the rat is sacrificed, the caval vein is extirpated, the thrombus harvested and its wet weight determined.

General Experimental Details

Where Prep-HPLC is stated, either a Waters Fraction Lynx Purification System with a ACE C8 5 μm 21×100 mm column or a Gilson HPLC System with a kromasil C8 10 μm 21.2×250 mm column was used. The mobile phase used with the Waters system was a gradient starting at 5% acetonitrile up to 100% in 100 mM ammonium acetate buffer. The mobile phase used with the Gilson system was a gradient starting at 0% acetonitrile up to 95% in 100 mM ammonium acetate buffer. The flow rate was 25 mL/minute. With the Waters system, MS triggered fraction collection was used. With the Gilson HPLC system, UV triggered fraction collection was used.

Mass spectra were recorded on either a Micromass ZQ single quadrupole or a Micromass quattro micro, both equipped with a pneumatically assisted electrospay interface (LC-MS).

Reagents

The following lists of reagents were used in the Preparations and Examples below. Unless otherwise stated, each of these reagents is commercially available.

List 1

(a) Phenylmethanesulfonyl chloride
(b) Benzenesulfonyl chloride
(c) 4-Methoxybenzenesulfonyl chloride
(d) 2-Methoxy-4-methylbenzenesulfonyl chloride
(e) 3,4-Dichlorobenzenesulfonyl chloride
(f) 3-Methoxybenzenesulfonyl chloride
(g) 2,5-Dimethylbenzenesulfonyl chloride
(h) Naphthalene-1-sulfonyl chloride
(i) 2,4-Dimethoxybenzenesulfonyl chloride
(j) (4-Chlorophenyl)methanesulfonyl chloride
(k) 4-Ethylbenzenesulfonyl chloride
(l) 2,5-Dimethylthiophene-3-sulfonyl chloride
(m) 2,5-Dichlorobenzenesulfonyl chloride
(n) 2-Chloro-6-methylbenzenesulfonyl chloride
(o) 4-Chloro-2-fluorobenzenesulfonyl chloride
(p) Phenylacetaldehyde
(q) Benzaldehyde
(r) 2-Methoxynicotinaldehyde (obtainable as described in J. Org. Chem. 55, 69 (1990))
(s) 2,2-Difluoro-2-pyridin-2-ylethyl trifluoromethanesulfonate (obtainable as described in J. Med. Chem. 46, 461 (2003))
(t) 3-Methoxybenzaldehyde
(u) 6-Chloro-1,3-benzodioxole-5-carbaldehyde
(v) 5-Chloro-1,3-dimethyl-1H-pyrazole-4-carbaldehyde
(w) Nicotinaldehyde
(x) 2-(Aminomethyl)-4-chlorophenol
   (obtainable as described in J. Med. Chem. 23(12), 1414 (1980))
(y) 1-(5-Chloro-2-methylphenyl)methanamine
   List 2
(a) [(4-Aminomethylphenyl)iminomethyl]carbamic acid benzyl ester (obtainable as described in WO 94/29336)
(b) (5-Aminomethyl-6-methylpyridin-2-yl)carbamic acid tert-butyl ester (obtainable as described in WO 97/01338)
(c) (4-Aminomethylpyridin-2-yl)carbamic acid tert-butyl ester (obtainable as described in Preparation 3 below)
(d) (4-Bromomethylpyridin-2-yl)carbamic acid tert-butyl ester (obtainable as described in WO 00/66557)
(e) C-(3-Fluoro-4-methylpyridin-2-yl)methylamine (obtainable as described in WO 00/075134)
(f) (5-Aminomethylpyridin-2-yl)carbamic acid tert-butyl ester (obtainable as described in WO 97/01338)
(g) (2-Aminomethyl-4-chlorobenzyl)carbamic acid tert-butyl ester (obtainable as described in WO 02/050056)
(h) [N,N′-Di-(tert-butoxycarbonyl)]-2-aminoethoxyguanidine (obtainable as described in WO 99/55355)
(i) (5-Aminomethyl-6-methylpyridin-2-yl)carbamic acid tert-butyl ester (obtainable as described in WO 97/01338)
(j) [2-(1H-Tetrazol-1-yl)benzyl]amine (obtainable as described in WO 02/064211)
(k) tert-Butyl [2-(aminomethyl)benzyl]carbamate (obtainable as described in WO 02/057225)
(l) [5-Chloro-2-(1H-tetrazol-1-yl)benzyl]amine (obtainable as described in WO 02/064559)
(m) 2-[2-(Aminomethyl)-4-chlorophenoxy]-N-ethylacetamide (obtainable as described in WO 97/30708)
(n) tert-Butyl {2-[2-(aminomethyl)-4-chlorophenyl]ethyl}carbamate (obtainable as described in Bioorg. Med. Chem. Lett., 13, 34773 (2003))
   Preparations
   Preparation 1

### (4-Methyl-1-nitroso-2-oxo-1,2,5,6-tetrahydropyridin-3-yl)acetic acid ethyl ester

### (a) 4-Methylpiperidine-1-carboxylic acid tert-butyl ester

4-Methylpiperidine (5.0 g, 50 mmol) and di-tert-butyl dicarbonate (13 g, 60 mmol) were dissolved in DCM (50 mL). TEA (7.65 mL, 1.1 mol equiv.) was added and the reaction mixture was stirred at 35° C. for 3 hours. The solvent was removed in vacuo and the residue was purified by flash chromatography (SiO₂, hexane) to give the sub-title compound (7.29 g, 73%).

¹H NMR (400 MHz, CDCl₃) δ 0.81 (d, 3H), 0.86-1.00 (m, 2H), 1.33 (s, 9H), 1.13-1.49 (m, 3H), 2.55 (m, 2H), 3.93 (m, 2H)

### (b) 4-Methyl-2-oxopiperidine-1-carboxylic acid tert-butyl ester

4-Methyl-piperidine-1-carboxylic acid tert-butyl ester (1.1 g, 5.5 mmol; see step (a) above) was dissolved in ethyl acetate (70 mL) and was added to a solution of ruthenium oxide (0.020 g, 0.15 mmol) and sodium periodate (4.5 g, 21 mmol) dissolved in water (215 mL). The reaction was stirred vigorously under air for 18 hours. The layers were separated and the aqueous phase was extracted with ethyl acetate. The combined organic extracts were dried and filtered through Celite®. The solvent was removed in vacuo and the residue (the sub-title compound—0.98 g, 83%) was used without further purification.

¹H NMR (400 MHz, CDCl₃) δ 1.02 (d, 3H), 1.43-1.57 (m, 1H), 1.53 (s, 9H), 1.90-2.03 (m, 2H), 2.04-2.30 (m, 1H), 2.56-2.62 (m, 1H), 3.46-3.53 (m, 1H), 3.78-3.82 (m, 1H)

### (c) 3-Ethoxycarbonylmethyl-4-methyl-2-oxopiperidine-1-carboxylic acid tert-butyl ester

Lithium bis(trimethylsilyl)amide (2.1 mL, 1 M in THF, 2.1 mmol) was added slowly to a solution of 4-methyl-2-oxopiperidine-1-carboxylic acid tert-butyl ester (0.40 g, 1.87 mmol; see step (b) above) in THF (7 mL) at −78° C. The solution was stirred for 40 minutes. Ethyl bromoacetate (0.31 mL, 2.8 mmol, 1.5 mol equiv.) was added at −78° C. and the reaction mixture was warmed to −20° C. over a period of 2 hours. The reaction was quenched by addition of ammonium chloride (sat., 10 mL). The mixture was diluted with ethyl acetate (30 mL) and the layers were separated. The aqueous phase was extracted with ethyl acetate (3×25 mL). The combined organic layers were dried (Na₂SO₄), filtered and concentrated under reduced pressure. Purification by flash chromatography (SiO₂, 10-20% ethyl acetate in hexane) gave the subtitle compound (0.387 g, 69%) as a colourless oil.

¹H NMR (400 MHz, CDCl₃) δ 0.95 (d, 3H) 1.15 (t, 3H), 1.33-1.47 (m, 1H), 1.41 (s, 9H), 1.79-1.93 (m, 2H), 2.29-2.34 (m, 1H), 2.59 (dd, 1H), 2.69 (dd, 1H), 3.51-3.56 (m, 1H), 3.57-3.67 (m, 1H), 4.03 (q, 2H)

### (d) 5-Ethoxycarbonylmethyl-4-methyl-6-oxo-3,6-dihydro-2H-pyridine-1-carboxylic acid tert-butyl ester

Lithium bis(trimethylsilyl)amide (3.1 mL, 1 M in THF, 3.1 mmol) was added slowly to a solution of 3-ethoxycarbonylmethyl-4-methyl-2-oxo-piperidine-1-carboxylic acid tert-butyl ester (0.77 g, 2.6 mmol; see step (c) above) in THF (26 mL) at −78° C. The solution was stirred for 90 minutes and then phenylselenium bromide (0.80 g, 3.4 mmol) in THF (2×3 mL) was added at −78° C. The reaction mixture was stirred at −78° C. for 90 minutes and was then warmed to −20° C. over a period of 2 hours and quenched by addition of ammonium chloride (sat., 60 mL). The mixture was diluted with ethyl acetate (50 mL) and the layers were separated. The aqueous phase was extracted with ethyl acetate (3×25 mL). The combined organic layers were dried (Na₂SO₄), filtered and concentrated under reduced pressure.

The residue was dissolved in DCM (10 mL) and cooled to 0° C. Hydrogen peroxide (30%, 10 mL) was added and the pH was adjusted to ˜7 with pyridine. The reaction mixture was allowed to warm to room temperature. The reaction mixture was quenched after 10 minutes at 0° C. with ammonium chloride (sat., 60 mL) and the mixture was extracted with DCM (50 mL). The organic phase was washed with brine, dried and the solvent was removed in vacuo. Purification and separation by flash chromatography (SiO₂, 20-60% ethyl acetate/hexane) gave the endocyclic compound (the sub-title compound—0.387 g, 69%) and the exocyclic compound as colourless oils.

The endocyclic compound was used in the next step.

Endocyclic Compound:

¹H NMR (400 MHz, CDCl₃) δ 1.24 (t, 3H), 1.52 (s, 9H), 1.93 (s, 3H), 2.41 (t, 2H), 3.40 (br s, 2H), 3.81 (t, 2H), 4.12 (q, 2H)

### (e) (4-Methyl-2-oxo-1,2,5,6-tetrahydropyridin-3-yl)acetic acid ethyl ester

TFA (0.1 mL, 0.1 volume equiv.) was added to a solution of 5-ethoxy-carbonylmethyl-4-methyl-6-oxo-3,6-dihydro-2H-pyridine-1-carboxylic acid tert-butyl ester (0.025 g, 0.084 mmol; see step (d) above) in DCM (1 mL) and the reaction was stirred for 4 hours at room temperature. The TFA was removed under reduced pressure azeotropically with benzene (3×20 mL) to give the sub-title compound (deprotected amine), which was used in the next step without further purification.

### (f) (4-Methyl-1-nitroso-2-oxo-12,5,6-tetrahydropyridin-3-yl)acetic acid ethyl ester

The title compound was prepared from the compound of step (e) above by one of the following two methods.

Method A

tert-Butyl nitrite (0.015 mL, 0.13 mmol, 1.5 mol equiv.) and pyridine (0.020 mL, 0.25 mmol, 3 mol equiv.) were added to the solution of the crude amine (from step (e) above) in dry diethyl ether (1 mL). The reaction mixture was heated to reflux for 16 hours. An additional aliquot of tert-butyl nitrite (0.010 mL, 0.084 mmol, 1 mol equiv.) was added and reflux was continued for 16 hours. The solvent was removed under reduced pressure and purification by flash chromatography (SiO₂, 50% ethyl acetate in hexane) gave the title compound (0.0174 g, 91%) as a yellow oil.

Method B

The crude amine (738 mg, 3.74 mmol; from step (e) above) was dissolved in water (7 mL) and dimethoxyethane (3.5 mL). Hydrochloric acid (0.7 mL, conc.) was added and the mixture was cooled to 0° C. Sodium nitrite (309 mg, 4.49 mmol) dissolved in water (3.5 mL) was added in portions of 600 mL, and the reaction mixture was stirred whilst gradually warming to room temperature. After 2.5 hours, another portion of sodium nitrite (36 mg) in water (1 mL) was added and stirring was continued for 45 minutes. The reaction mixture was extracted with DCM and the organic phase was dried through a phase separator. The solvent was evaporated under reduced pressure and purification by flash chromatography (SiO₂, hexane:ethyl acetate 2.1) gave the title (535 mg, 63%)

¹H NMR (400 MHz, CDCl₃) δ 1.30 (t, 3H), 2.08 (s, 3H), 2.57 (t, 2H), 3.59 (s, 2H), 3.89 (t, 2H), 4.20 (q, 2H)

Preparation 2

The compounds (i) to (ix) listed below were prepared from the compound of Preparation 1 by step (i) method A and then step (ii) method A.

The compound (x) listed below was prepared from the compound of Preparation 1 by step (i) method B and then step (ii) method B.

The compound (xi) listed below was prepared from the compound of Preparation 1 by step (i) method A and then step (ii) method B.

The compounds (xii) to (xviii) below were prepared from the compound of Preparation 1 by the methods described below.

Step (i)

Method A

Zinc powder (0.014 g, 0.21 mmol, 3 mol equiv.) was added to a solution of (4-Methyl-1-nitroso-2-oxo-1,2,5,6-tetrahydropyridin-3-yl)acetic acid ethyl ester (0.016 g, 0.071 mmol; see Preparation 1 above) in a mixture of methanol and acetic acid (2 mL, 1:1) at 0° C. The ice bath was removed and after approximately 5 to 10 minutes the yellow colour had disappeared. The reaction mixture was filtered through Celite® and the filter cake was washed with methanol (3×5 mL). The solvent was removed under reduced pressure and the excess acetic acid was removed azeotropically with benzene (3×5 mL) to give the reduced intermediate which was used without further purification in step (ii) below.

Method B

(4-Methyl-1-nitroso-2-oxo-1,2,5,6-tetrahydropyridin-3-yl)acetic acid ethyl ester (200 mg, 0.884 mmol; see Preparation 1 above) was divided equally into four vials which were treated exactly the same. Water (2.5 mL), ammonium acetate buffer (7.5 mL, 4 M) and acetonitrile (1.5 mL) were added and the mixture was cooled to 0° C. A solution of TiCl₃ (2.0 mL of a 13% solution in 20% HCl (aq.)) was added and the reaction mixture was stirred at 0° C. for 30 minutes. The four mixtures were collected and the acetonitrile was evaporated under reduced pressure. The residue was extracted with DCM (3×) and the organic phase was dried through a phase separator. The solvent was evaporated under reduced pressure to give the crude product (187 mg, 65%), which was used directly without purification.

Step (ii)

Method A

The specific sulfonyl chloride (0.11 mmol, 1.2 mol equiv; see List 1 above) and pyridine (0.018 mL, 0.22 mmol, 3 mol equiv.) was added to a solution of (1-amino-4-methyl-2-oxo-1,2,5,6-tetrahydropyridin-3-yl)acetic acid ethyl ester (0.071 mmol; see step (i) above) in DCM (2 mL) at 0° C. The reaction mixture was stirred at room temperature for 16 hours. Pyridine and the solvent were evaporated under reduced pressure. Purification by flash chromatography (SiO₂, 50-70% ethyl acetate in hexane) gave the sulfonamides listed at (i) to (ix) below (52-91%, over two steps).

Method B

The crude 1-amino-4-methyl-2-oxo-1,2,5,6-tetrahydropyridin-3-yl)acetic acid ethyl (187 mg, 0.57 mmol; see step (i) above) was dissolved in methanol (1.5 mL) and the specific aldehyde (0.57 mmol, 1 eq.; see List 1 above) dissolved in methanol (1 mL) was added. Zinc chloride (195 mg, 1.43 mmol) in methanol (2 mL) and sodium cyanoborohydride (63 mg, 2.85 mmol) were added. The reaction mixture was stirred at room temperature overnight. The reaction mixture was then partitioned between sodium hydrogencarbonate (sat.) and DCM. The mixture was extracted with DCM (3×) and the organic phase was dried through a phase separator and the solvent was evaporated under reduced pressure. Purification by flash chromatography (SiO₂, ethyl acetate:heptane, 1:1) gave the compounds listed at (x) and (xi) below (55%).

### (i) (4-Methyl-2-oxo 1-phenylmethanesulfonylamino-1,2,5,6-tetraydropyridin-3-yl)acetic acid ethyl ester

¹H NMR (400 MHz, CDCl₃) δ 1.27 (t, 3H), 1.97 (s, 3H), 2.58 (t, 2H), 3.43 (s, 2H), 3.79 (t, 2H), 4.16 (q, 2H), 4.32 (s, 2H), 7.27-7.37 (m, 3H), 7.39-7.44 (m, 2H), 7.61 (br s, 1H)

### (ii) [1-(3-Methoxybenzenesulfonylamino)-4-methyl-2-oxo-1,2,5,6-tetrahydropyridin-3-yl]acetic acid ethyl ester

¹H NMR (400 MHz, CDCl₃) δ 1.18 (t, 3H), 1.90 (s, 3H), 2.59 (t, 2H), 3.17 (s, 2H), 3.83 (s, 5H), 4.03 (q, 2H), 7.08 (dd, 1H), 7.37 (t, 2H), 7.48 (dd, 1H), 7.75 (s, 1H)

### (iii) [4-Methyl-1-(naphthalene-1-sulfonylamino)-2-oxo-1,2,5,6-tetrahydropyridin-3-yl]acetic acid ethyl ester

¹H NMR (400 MHz, CDCl₃) δ δ 1.10 (t, 3H), 1.84 (s, 3H), 2.53 (t, 2H), 3.02 (s, 2H), 3.75 (t, 2H), 3.94 (q, 2H), 7.52 (t, 1H), 7.60 (t, 1H), 7.68 (t, 1H), 7.89 (m, 2H), 8.07 (d, 1H), 8.27 (d, 1H), 8.32 (d, 1H)

### (iv) [1-(2,5-Dimethylbenzenesulfonylamino)-4-methyl-2-oxo-1,2,5,6-tetrahydropyridin-3-yl]acetic acid ethyl ester

¹H NMR (400 MHz, CDCl₃) δ 1.17 (t, 3H), 1.88 (s, 3H), 2.33 (s, 3H), 2.52 (t, 2H), 2.70 (s, 3H), 3.19 (s, 2H), 3.76 (t, 2H), 4.04 (q, 2H), 7.15 (d, 1H), 7.76 (d, 2H)

### (v) [1-(2,5-Dichlorobenzenesulfonylamino)-4-methyl-2-oxo-1,2,5,6-tetrahydropyridin-3-yl]acetic acid ethyl ester

¹H NMR (400 MHz, CDCl₃) δ 1.17 (t, 3H), 1.88 (s, 3H), 2.57 (t, 2H), 3.20 (s, 2H), 3.81 (t, 2H), 4.01 (q, 2H), 7.44 (s, 2H), 7.98 (s, 1H), 8.25 (s, 1H)

### (vi) [1-(2-Methoxy-4-methylbenzenesulfonylamino)-4-methyl-2-oxo-1,2,5,6-tetrahydropyridin-3-yl]acetic acid ethyl ester

¹H NMR (400 MHz, CDCl₃) δ 1.14 (t, 3H), 1.83 (s, 3H), 2.36 (s, 3H), 2.48 (t, 2H), 3.17 (s, 2H), 3.78 (t, 2H), 3.97-4.02 (m, 5H), 6.76 (s, 2H), 7.67 (d, 1H), 8.43 (s, 1H)

### (vii) [1-(2-Chloro-6-methylbenzenesulfonylamino)-4-methyl-2-oxo-1,2,5,6-tetrahydropyridin-3-yl]acetic acid ethyl ester

¹H NMR (400 MHz, CDCl₃) δ 1.14 (t, 3H), 1.87 (s, 3H), 2.56 (t, 2H), 2.70 (s, 3H), 3.19 (s, 2H), 3.83 (t, 2H), 4.00 (q, 2H), 7.13 (d, 1H), 7.29 (d, 1H), 7.38 (d, 1H), 8.46 (s, 1H)

### (viii) [1-(4-Chloro-2-fluorobenzenesulfonylamino)-4-methyl-2-oxo-1,2,5,6-tetrahydropyridin-3-yl]acetic acid ethyl ester

¹H NMR (400 MHz, CDCl₃) δ 1.18 (t, 3H), 1.88 (s, 3H), 2.57 (t, 2H), 3.18 (s, 2H), 3.80 (t, 2H), 4.02 (q, 2H), 7.20 (d, 2H), 7.77 (d, 1H), 7.99 (s, 1H)

### (ix) (1-Benzenesulfonylamino-4-methyl-2-oxo-1,2,5,6-tetrahydro-pyridin-3-yl)acetic acid ethyl ester

¹H NMR (400 MHz, CDCl₃) δ 1.16 (t, 3H), 1.83 (s, 3H), 2.59 (t, 2H), 3.14 (s, 2H), 3.82 (t, 2H), 4.01 (q, 2H), 7.47 (t, 2H), 7.57 (t, 1H), 7.73 (s, 1H), 7.88 (d, 2H)

### (x) Ethyl {4-methyl-2-oxo-1-[(2-phenylethyl)amino]-1,2,5,6-tetrahydropyridin-3-yl}acetate

¹H NMR (500 MHz, CDCl₃) d 1.27 (t, 3H), 1.91 (s, 3H), 2.45 (t, 2H), 2.83 (t, 2H), 3.14 (t, 2H), 3.42 (s, 2H), 3.52 (t, 2H), 4.16 (t, 2H), 7.19-7.33 (m, 5H)

MS m/z 317 (M+H)⁺

### (xi) Ethyl [1-(benzylamino)-4-methyl-2-oxo-1,2,5,6-tetrahydropyridin-3-yl]acetate

The compound was prepared following Preparation 2, step (i), Method A, except that the reduced crude material was partitioned between sodium hydrogen carbonate (sat.) and DCM. The mixture was extracted with DCM (3×) and the organic phase was dried through a phase separator. The solvent was evaporated under reduced pressure to give the reduced intermediate, which was used without further purification in step (ii), Method B above. After stirring overnight, acetic acid (6 eq.) was added to facilitate reduction of the intermediate imine.

¹H NMR (500 MHz, CDCl₃) d 1.26 (t, 3H), 1.86 (s, 3H), 2.30 (t, 2H), 3.34 (t, 2H), 3.40 (s, 2H), 3.97 (s, 2H), 4.14 (q, 2H), 5.51 (br s, 1H), 7.25-7.40 (m, 5H)

### (xii) Ethyl (1-{[(2-methoxypyridin-3-yl)methyl]amino}-4-methyl-2-oxo-1,2,5,6-tetrahydropyridin-3-yl)acetate

Reduction performed according to step (i) method A, except that excess acetic acid was removed by washing with basic water. Reductive amination performed according to step (ii) method B, except that NaBH₃CN and acetic acid were added after stirring for one night. Further aldehyde (0.25 equiv.), NaBH₃CN (6 equiv.) and acetic acid (12 droplets) were then added and the reaction mixture was stirred for another 2 hours. Purification by flash chromatography (SiO₂, 0.25% methanol in DCM+1% TEA) and Prep-HPLC gave the title compound in 45% yield.

¹H NMR (500 MHz, CDCl₃) δ 1.27 (t, 3H), 1.89 (s, 3H), 2.41 (t, 2H), 3.41 (s, 2H), 3.46 (t, 2H), 3.99 (s, 2H), 4.00 (s, 3H), 4.15 (q, 2H), 6.85 (dd, 1H), 7.57 (dd, 1H), 8.11 (dd, 1H)

MS m/z 334 (M+H)⁺

### (xiii) Ethyl {1-[(3-methoxybenzyl)amino]-4-methyl-2-oxo-1,2,5,6-tetrahydropyridin-3-yl}acetate

Prepared according to the procedure outlined in Preparation 2 (xii) above.

¹H NMR (500 MHz, CDCl₃) δ 1.24 (t, 3H), 1.85 (s, 3H), 2.30 (t, 2H), 3.35 (t, 2H), 3.39 (s, 2H), 3.78 (s, 3H), 3.94 (s, 2H), 4.13 (q, 2H), 6.80 (d, 1H), 6.93 (s, 1H), 6.95 (d, 1H), 7.21 (t, 1H)

MS m/z 333.31 (M+H)⁺

### (xiv) Ethyl (1-{[(6-chloro-1,3-benzodioxol-5-yl)methyl]amino}-4-methyl-2-oxo-1,2,5,6-tetrahydropyridin-3-yl)acetate

Prepared according to the procedure outlined in Preparation 2 (xii) above.

¹H NMR (500 MHz, CDCl₃) δ 1.23 (t, 3H), 1.87 (s, 3H), 2.39 (t, 2H), 3.38 (s, 2H), 3.42 (t, 2H), 4.00 (s, 2H), 4.12 (q, 2H), 5.94 (s, 2H), 6.81 (s, 1H), 6.88 (s, 1H)

MS m/z 380.96 (M+H)⁺

### (xv) Ethyl (1-{[(5-chloro-1,3-dimethyl-1H-pyrazol-4-yl)methyl]amino}-4-methyl-2-oxo-1,2,5,6-tetrahydropyridin-3-yl)acetate

Prepared according to the procedure outlined in Preparation 2 (xii) above.

¹H NMR (500 MHz, CDCl₃) δ 1.26 (t, 3H), 1.89 (s, 3H), 2.29 (s, 3H), 2.38 (t, 2H), 3.38-3.44 (m, 4H), 3.78 (s, 3H), 3.83 (s, 2H), 4.15 (q, 2H), 5.34 (br. s, 1H)

### (xvi) Ethyl {4-methyl-2-oxo-1-[(pyridin-3-ylmethyl)amino]-1,2,5,6-tetrahydropyridin-3-yl}acetate

Prepared according to the procedure outlined in Preparation 2 (xii) above.

¹H NMR (500 MHz, CDCl₃) δ 1.27 (t, 3H), 1.88 (s, 3H), 2.33 (t, 2H), 3.34 (t, 2H), 3.40 (s, 2H), 4.01 (d, 2H), 4.16 (q, 2H), 5.52 (t, 1H), 7.25-7.30 (m, 1H), 7.71-7.78 (m, 1H), 8.52-8.56 (m, 1H), 8.60-8.63 (m, 1H)

### (xvii) Ethyl {1-[(2,2-difluoro-2-pyridin-2-ylethyl)amino]-4-methyl-2-oxo-1,2,5,6-tetrahydropyridin-3-yl}acetate

Reduction performed according to step (i) method A. Coupling performed is by stirring a solution of the amine produced in step (i) and 2,2-difluoro-2-pyridin-2-ylethyl trifluoromethanesulfonate (0.88 equiv.; see List 1 above) in 1,2-dichloroethane for 3 days at 50° C. Solvent was then removed and the product purified by flash chromatography (SiO₂, ethyl acetate) to give the title compound.

¹H NMR (500 MHz, CDCl₃) δ 8.67 (d, 1H), 7.82 (dt, 1H), 7.69 (d, 1H), 7.38 (dd, 1H), 5.51 (t, 1H), 4.14 (q, 2H), 3.71-3.81 (m, 2H), 3.47 (t, 2H), 3.38 (s, 2H), 2.41 (t, 2H), 1.88 (s, 3H), 1.26 (t, 3H)

### (xviii) Ethyl (1-{[(5-chloro-2-thienyl)methyl]amino}-4-methyl-2-oxo-1,2,5,6-tetrahydropyridin-3-yl)acetate

Prepared according to the procedure outlined in Preparation 2 (xii) above.

¹H NMR (500 MHz, CDCl₃) δ 1.26 (t, 3H), 1.89 (s, 3H), 2.38 (t, 2H), 3.38-3.45 (m, 4H), 4.09 (s, 2H), 4.15 (q, 2H), 5.50 (br s, 1H), 6.73-6.78 (m, 2H)

MS m/z 343 (M+H)⁺

Preparation 3

### (4-Aminomethylpyridin-2-yl)carbamic acid tert-butyl ester

### (a) (4-Azidomethyl-pyridin-2-yl)-carbamic acid tert-butyl ester

A mixture of (4-bromomethylpyridin-2-yl)carbamic acid tert-butyl ester (3.0 g, 0.010 mol; obtainable as described in WO 00/66557) and sodium azide (1.36 g, 0.0209 mol) in water (20 mL) and DMF (40 mL) was stirred overnight. The reaction mixture was poured into water (300 mL) and extracted with ethyl acetate (3×). The combined organic phases were washed with water, dried (Na₂SO₄), filtered and the solvent was evaporated under reduced pressure. The crude product crystallised (2.6 g, 100%) and was used without further purification.

¹H NMR (300 MHz, CDCl₃) δ 10.14 (bs, 1H), 8.36 (d, 1H), 7.99 (bs, 1H), 6.91 (m, 1H), 4.37 (bs, 2H), 1.54 (s, 9H)

### (b) (4-Aminomethylpyridin-2-yl)carbamic acid tert-butyl ester

A solution of sodium borohydride (0.92 g, 24 mmol) in water (25 mL) was added to a slurry of Pd/C (10%, 50 mg) in water (25 mL) under stirring. Next, (4-azidomethylpyridin-2-yl)carbamic acid tert-butyl ester (0.40 g, 6.1 mmol; see step (a) above) in THF (75 mL) was added dropwise rather rapidly under ice-cooling. The reaction was stirred at room temperature for 4 hours. An aqueous solution of sodium hydrogensulfate was added slowly to give an acidic pH. The reaction mixture was suction filtered through a Celite® pad which was further washed with water. The combined aqueous layer was washed with ethyl acetate, made alkaline by addition of NaOH (aq.) and extracted with ethyl acetate (3×). The combined organic phases were washed with water, dried (Na₂SO₄), filtered and the solvent was evaporated under reduced pressure. The crude product (1.1 g, 85%) crystallised and was used without further purification.

¹H NMR (300 MHz, CDCl₃) δ 10.06 (m, 1H), 8.25 (m, 1H), 7.94 (m, 1H), 6.88 (m, 1H), 3.83 (bs, 2H), 1.50 (s, 9H).

Preparation 4

### [2-(Aminomethyl)-4-fluorophenyl]methanol

### (a) Methyl 2-bromo-4-fluorobenzoate

2-Bromo-4-fluorobenzoic acid (1.0 g, 4.6 mmol) was dissolved in methanol (3 mL) before methanol saturated with HCl (10 mL) was added. The reaction mixture was stirred overnight and then concentrated. The excess of HCl was removed by co-evaporation from methanol to give the sub-title compound (in 94% yield), which was used in the next step without further purification.

¹H NMR (500 MHz, CDCl₃) δ 3.92 (s, 3H), 7.04-7.12 (m, 1H), 7.38-7.44 (m, 1H), 7.83-7.91 (m, 1H)

### (b) Methyl 2-cyano-4-fluorobenzoate

Methyl 2-bromo-4-fluorobenzoate (1.0 g, 4.29 mmol; see step (a) above) was dissolved in dry DMF (5 mL) and degassed with N₂-gas for 5 minutes. CuCN (769 mg, 8.58 mmol) was added and mixture was degassed again before the temperature was raised. The reaction mixture was refluxed for 75 minutes. NaCN (aq, 10%) was added and the mixture was extracted with DCM. The DCM phase was dried through a phase separator and the solvent was removed in vacuo. The crude product was dissolved in toluene and washed once with water. The organic phase was dried over MgSO₄ and filtered. The solvent was removed in vacuo to give the product (in 94% yield), which was used without ether purification.

¹H NMR (500 MHz, CDCl₃) δ 4.01 (s, 3H), 7.35-7.42 (m, 1H), 7.52 (dd, 1H), 8.15-8.23 (m, 1H)

### (c) [2-(Aminomethyl)-4-fluorophenyl]methanol

LiAlH₄ (357 g, 9.41 mmol) was suspended in dry THF (5 mL) and the resulting mixture cooled with an ice bath. Methyl 2-cyano-4-fluorobenzoate (562 mg, 3.14 mmol; see step (b) above) was dissolved in THF (5+3 mL) and added to the reducing agent. The reaction mixture was stirred for 10 minutes and then the ice bath was removed. After 2 hours, the reaction was quenched with water (2 mL), NaOH (2 M, 2 mL) and more water (2 mL) and the resulting mixture was stirred for 10 minutes. The mixture was diluted with diethyl ether (30 mL) and filtered. The organic phase was dried over MgSO₄ and filtered. The solvent was removed in vacuo and the residue was then purified by Prep-HPLC, which provided the title compound in 16% yield.

¹H NMR (500 MHz, CDCl₃) δ 4.25 (s, 2H), 4.74 (s, 2H), 7.14-7.20 (m, 1H), 7.28 (dd, 1H), 7.43-7.48 (m, 1H)

Preparation 5

### [2-(Aminomethyl)-4-chlorophenyl]methanol

The title compound was prepared by analogy with the methods described in Preparation 4 above, with 2-bromo-4-chlorobenzoic acid being used in place of 2-bromo-4-fluorobenzoic acid.

¹H NMR (500 MHz, MeOD) δ 4.23 (s, 2H), 4.77 (s, 2H), 7.40-7.47 (m, 2H), 7.53-7.57 (m, 1H)

### EXAMPLES

### Example 1

The compounds (i) to (ix) listed below were prepared from corresponding compounds of Preparation 2 by the hydrolysis of step (i), followed by amide coupling as in step (ii), Method A.

Unless otherwise stated, the compounds (x) to (xxiii) listed below were prepared from corresponding compounds of Preparation 2 by the hydrolysis of step (i), followed by amide coupling as in step (ii), Method B.

Unless otherwise stated, the compounds (xxiv) to (l) listed below were prepared from corresponding compounds of Preparation 2 by the hydrolysis described in Example 1(xxvi), followed by amide coupling as described in Example 1(xxiii).

Step (i)

Lithium hydroxide (1.5 mol equiv.) was added to the specific ester (0.041 mmol; see Preparation 2 above) dissolved in THF (1 mL) and water (3 drops). The reaction mixture was stirred at room temperature for 11 hours and then quenched with water (10 mL). The mixture was acidified (HCl, 1 M) to pH ˜3 and the solution was extracted with ethyl acetate (3×10 mL). The combined organic phases were dried and the solvent was removed under reduced pressure. The residue (the carboxylic acid) was used without further purification in the next reaction.

Step (ii)

Method A

The specific carboxylic acid (see step (i) above), the specific amine (2 mol equiv.; see List 2 above) and HOBT (2 mol equiv.) were dissolved in DMF (0.7 mL) and the solution was cooled to 0° C. DIPEA (4 mol equiv.) and EDC (2 mol equiv.) were added and the reaction mixture was stirred at room temperature under an atmosphere of argon for 72 h. DMF was removed under reduced pressure and purification by flash chromatography (SiO₂, ethyl acetate) gave the amides listed at (i) to (ix) below as oils (57-85%).

Method B

The crude specific carboxylic acid (see step (i) above) was dissolved in DCM (5 mL) and TEA (2 equiv.), and the specific amine (1 equiv.; see List 2 above) was added. The mixture was cooled to 0° C. and PyBOP (1 equiv.) was added. The reaction mixture was stirred at 0° C. for 30 minutes and then allowed to warm to room temperature and was further stirred overnight. The solvent was removed under reduced pressure and the residue was purified by chromatography (SiO₂, hexane:ethyl acetate 1:2) to give the product (68%).

### (i) [Amino-(4-{[2-(4-methyl-2-oxo-1-phenylmethanesulfonylamino-1,2,5,6-tetrahydropyridin-3-yl)acetylamino]methyl}phenyl)methylene]-carbamic acid benzyl ester

¹H NMR (400 MHz, CDCl₃) δ 2.06 (br d, 3H), 2.51 (br t, 2H), 3.34 (br s, 2H), 3.58 (br t, 2H), 4.23 (s, 2H), 4.40 (br s, 2H), 5.21 (s, 2H), 7.08-7.45 (m, 14H), 7.65 (d, 2H), 7.37 (br s, 1H), 7.39 (br s, 1H)

### (ii) {Amino-[4-({2-[1-(3-methoxybenzenesulfonylamino)-4-methyl-2-oxo-1,2,5,6-tetrahydropyridin-3-yl]acetylamino}methyl)phenyl]-methylene}carbamic acid benzyl ester

¹H NMR (400 MHz, CDCl₃) δ 2.00 (s, 3H), 2.53 (t, 2H), 3.11 (s, 2H), 3.73 (s, 5H), 4.21 (d, 2H), 5.20 (s, 2H), 6.69 (t, 1H), 6.99 (dd, 1H), 7.12 (d, 2H), 7.23-7.36 (m, 6H), 7.42 (t, 3H), 7.76 (d, 2H)

### (iii) {Amino-[4-({2-[4-methyl-1-(naphthalene-1-sulfonylamino)-2-oxo-1,2,5,6-tetrahydropyridin-3-yl]acetylamino}methyl)phenyl]methylene}-carbamic acid benzyl ester

¹H NMR (400 MHz, CDCl₃) δ 1.95 (s, 3H), 2.44 (t, 2H), 3.01 (s, 2H), 3.57 (t, 2H), 4.06-4.15 (m, 2H), 5.22 (s, 2H), 6.54 (t, 1H), 7.07 (d, 2H), 7.30-7.38 (m, 3H), 7.41-7.46 (m, 3H), 7.54 (t, 1H), 7.64 (t, 1H), 7.71 (d, 2H), 7.80 (d, 1H), 7.96 (d, 1H), 8.23 (d, 1H), 8.77 (d, 1H)

### (iv) {Amino-[4-({2-[1-(2,5-dimethylbenzenesulfonylamino)-4-methyl-2-oxo-1,2,5,6-tetrahydropyridin-3-yl]acetylamino}methyl)phenyl]-methylene}carbamic acid benzyl ester

¹H NMR (400 MHz, CDCl₃) δ 2.00 (s, 3H), 2.29 (s, 3H), 2.47 (t, 2H), 2.66 (s, 3H), 3.17 (s, 2H), 3.62 (t, 2H), 4.23 (d, 2H), 5.22 (s, 2H), 6.77 (t, 1H), 7.07 (d, 1H), 7.19 (d, 3H), 7.27-7.38 (m, 3H), 7.44 (d, 2H), 7.77 (d, 3H)

### (v) {Amino-[4-({2-[1-(2,5-dichlorobenzenesulfonylamino)-4-methyl-2-oxo-1,2,5,6-tetrahydropyridin-3-yl]acetylamino}methyl)phenyl]-methylene}carbamic acid benzyl ester

¹H NMR (400 MHz, CDCl₃) δ 1.92 (s, 3H), 2.50 (t, 2H), 3.09 (s, 2H), 3.69 (t, 2H), 4.20 (t, 2H), 5.15 (s, 2H), 6.96 (t, 1H), 7.14 (d, 2H), 7.22-7.32 (m, 5H), 7.38 (d, 2H), 7.70 (d, 2H), 7.91 (s, 1H)

### (vi) Amino-[4-({2-[1-(2-methoxy-4-methylbenzenesulfonylamino)-4-methyl-2-oxo-1,2,5,6-tetrahydropyridin-3-yl]acetylamino}methyl)phenyl]-methylene}carbamic acid benzyl ester

¹H NMR-(400 MHz, CDCl₃) δ 1.94 (s, 3H), 2.23 (s, 3H), 2.48 (s, 2H), 3.12 (s, 2H), 3.75 (t, 2H), 3.94 (s, 3H), 4.13 (d, 2H), 5.19 (s, 2H), 6.64 (t, 2H), 6.69 (s, 1H), 7.08 (d, 2H), 7.29-7.36 (m, 2H), 7.42 (d, 2H), 7.61 (d, 2H), 7.80 (d, 2H), 8.39 (br, 1H), 9.21 (br, 1H)

### (vii) {Amino-[4-({2-[1-(2-chloro-6-methylbenzenesulfonylamino)-4-methyl-2-oxo-1,2,5,6-tetrahydropyridin-3-yl]acetylamino}methyl)phenyl]-methylene}carbamic acid benzyl ester

¹H NMR (400 MHz, CDCl₃) δ 1.93 (s, 3H), 2.52 (t, 2H), 2.62 (s, 3H), 3.13 (s, 2H), 3.77 (t, 2H), 4.16 (t, 2H), 5.20 (s, 2H), 6.56 (t, 1H), 6.96 (d, 1H), 7.09 (d, 2H), 7.15 (t, 1H), 7.26-7.31 (m, 3H), 7.34 (t, 2H), 7.42 (d, 2H), 7.78 (d, 2H)

### (viii) {Amino-[4-({2-[1-(4-chloro-2-fluorobenzenesulfonylamino)-4-methyl-2-oxo-1,2,5,6-tetrahydropyridin-3-yl]acetylamino}methyl)phenyl]-methylene}carbamic acid benzyl ester

¹H NMR (400 M, CDCl₃) δ 1.94 (s, 3H), 2.59 (m, 2H), 3.20 (s, 2H), 3.73 (t, 2H), 4.40 (s, 2H), 5.42 (s, 2H), 7.28 (dd, 1H), 7.35 (dd, 1H), 7.39-7.44 (m, 3H), 7.53 (d, 4H), 7.77 (d, 2H), 7.82 (t, 1H)

### (ix) [Amino-(4-{[2-(1-benzenesulfonylamino-4-methyl-2-oxo-1,2,5,6-tetrahydropyridin-3-yl)acetylamino]methyl}phenyl)methylene]carbamic acid benzyl ester

¹H NMR (400 MHz, CDCl₃) δ 2.00 (s, 3H), 2.54 (t, 2H), 3.10 (s, 2H), 3.72 (t, 2H), 4.20 (d, 2H), 5.21 (s, 2H), 6.67-6.68 (m, 1H), 7.13 (d, 2H), 7.29-7.39 (m, 7H), 7.43-7.48 (m, 3H), 7.75 (d, 2H), 7.84 (d, 2H)

### (x) tert-Butyl (2-{[({1-[(benzylsulfonyl)amino]-4-methyl-2-oxo-1,2,5,6-tetrahydropyridin-3-yl}acetyl)amino]methyl}-4-chlorobenzyl)carbamate

The ester was hydrolysed according to step (i) above, except that 3 equivalents of lithium hydroxide was added, the volume of solvent was 2 mL, and the reaction mixture was stirred overnight.

¹H NMR (500 MHz, CD₃OD) δ 1.40 (s, 9H), 2.02 (s, 3H), 2.64 (t, 2H), 3.42 (s, 2H), 3.73 (t, 2H), 4.26 (s, 2H), 4.35 (s, 2H), 4.41 (s, 2H), 7.13 (d, 1H), 7.20 (d, 1H), 7.32-7.37 (m, 4H), 7.37-7.42 (m, 2H)

MS m/z 591.8 (M+H)⁺

### (xi) tert-Butyl (5-{[({1-[(benzylsulfonyl)amino]-4-methyl-2-oxo-1,2,5,6-tetrahydropyridin-3-yl}acetyl)amino]methyl}-6-methylpyridin-2-yl)-carbamate

¹H NMR (500 MHz, CDCl₃) δ 1.52 (s, 9H), 2.13 (s, 3H), 2.37 (s, 3H), 2.56 (t, 2H), 3.33 (s, 2H), 3.73 (t, 2H), 4.21 (s, 2H), 4.33 (d, 2H), 6.63 (t, 1H), 7.13 (s, 1H), 7.33-7.40 (m, 5H), 7.43 (d, 1H), 7.61 (d, 1H), 7.70 (s, 1H)

MS m/z 558.1 (M+H)⁺

### (xii) tert-Butyl (6-methyl-5-{[({4-methyl-1-[(1-naphthylsulfonyl)amino]-2-oxo-1,2,5,6-tetrahydropyridin-3-yl}acetyl)amino]methyl}pyridin-2-yl)-carbamate

¹H NMR (500 MHz, CDCl₃) δ 8.76 (d, 1H), 8.25 (d, 1H), 8.16 (br s, 1H), 7.98 (d, 1H), 7.83 (d, 1H), 7.61-7.68 (m, 2H), 7.58 (t, 1H), 7.46 (t, 1H), 7.14 (t, 1H), 6.12 (t, 1H), 4.03 (d, 2H), 3.75 (t, 2H), 2.98 (s, 2H), 2.55 (t, 2H), 2.32 (s, 3H), 2.03 (s, 3H), 1.52 (s, 9H)

### (xiii) 2-{4-Methyl-1-[(1-naphthylsulfonyl)amino]-2-oxo-1,2,5,6-tetrahydropyridin-3-yl}-N-[2-(1H-tetrazol-1-yl)benzyl]acetamide

Synthesised via step (ii), Method A above, except that HOAT and TEA were used and that the product was purified with reverse phase HPLC.

¹H NMR (500 MHz, CD₃OD) δ 9.43 (s, 1H), 8.83 (d, 1H), 8.25 (d, 1H), 8.11 (d, 1H), 7.94 (d, 1H), 7.43-7.72 (m, 8H), 4.04 (s, 2H), 3.57 (t, 2H), 2.94 (s, 2H), 2.49 (t, 2H), 1.87 (s, 3H)

MS m/z 532 (M+H)⁺

### (xiv) tert-Butyl (2-{[({4-methyl-1-[(1-naphthylsulfonyl)amino]-2-oxo-1,2,5,6-tetrahydropyridin-3-yl}acetyl)amino]methyl}benzyl)carbamate

Synthesised via step (ii), Method A above, except that HOAT and TEA were used.

¹H NMR (500 MHz, CDCl₃) δ 8.82 (d, 1H), 8.26 (d, 1H), 7.98-8.14 (d, 2H), 7.85 (d, 1H), 7.68 (t, 1H), 7.58 (t, 1H), 7.46 (t, 1H), 7.31 (t, 1H), 7.22 (t, 1H), 7.01 (br s, 1H), 6.25 (br s, 1H), 4.27 (br s, 2H), 4.16 (s, 3H), 3.69 (t, 2H), 2.98 (s, 2H), 2.51 (t, 2H), 2.00 (s, 3H), 1.62 (br s, 2H), 1.49 (s, 9H)

### (xv) tert-Butyl (4-chloro-2-{[({4-methyl-2-oxo-1-[(phenylsulfonyl)amino]-1,2,5,6-tetrahydropyridin-3-yl}acetyl)amino]methyl}benzyl)carbamate

The ester hydrolysis was performed as described in respect of Example 1(x) above.

¹H NMR (500 MHz, CDCl₃) δ 1.45 (s, 9H), 2.04 (s, 3H), 2.61 (t, 2H), 3.10 (s, 2H), 3.81 (t, 2H), 4.19 (d, 2H), 4.22 (d, 2H), 5.09 (br s, 1H), 6.44 (br s, 1H), 6.94 (s, 1H), 7.20-7.25 (m, 2H), 7.34 (t, 2H), 7.46 (t, 1H), 7.87 (d, 2H)

### (xvi) Di-tert-butyl [(E)-({2-[({4-methyl-2-oxo-1-[(phenylsulfonyl)amino]-1,2,5,6-tetrahydropyridin-3-yl}acetyl)amino]ethoxy}amino)methylylidene]-biscarbamate

The ester hydrolysis was performed as described in respect of Example 1(x) above.

¹H NMR (500 MHz, CDCl₃) δ 1.46 (s, 9H), 1.52 (s, 9H), 1.98 (s, 3H), 2.56 (t, 2H), 3.10 (s, 2H), 3.36 (q, 2H), 3.81 (t, 2H), 3.97 (t, 2H), 6.98 (br s, 1H), 7.48 (t, 2H), 7.60 (t, 1H), 7.66 (br s, 1H), 7.81 (br s, 1H), 7.88 (d, 2H), 9.04 (s, 1H)

### (xvii) tert-Butyl (6-methyl-5-{[({4-methyl-2-oxo-1-[(phenylsulfonyl)-amino]-1,2,5,6-tetrahydropyridin-3-yl}acetyl)amino]methyl}pyridin-2-yl)-carbamate

The ester hydrolysis was performed as described in respect of Example 1(x) above.

¹H NMR (500 MHz, CDCl₃) δ 1.51 (s, 9H), 2.07 (s, 3H), 2.33 (s, 3H), 2.59 (t, 2H), 3.08 (s, 2H), 3.81 (t, 2H), 4.14 (d, 2H), 6.23 (br s, 1H), 7.27 (d, 1H), 7.40 (t, 2H), 7.49 (t, 1H), 7.66 (d, 1H), 7.85 (d, 2H), 7.91 (br s, 1H)

### (xviii) Di-tert-butyl [(E)-({2-[({4-methyl-2-oxo-1-[(2-phenylethyl)amino]-1,2,5,6-tetrahydropyridin-3-yl}acetyl)amino]ethoxy}amino)methylylidene]-biscarbamate

The ester was hydrolysed according to step (i) above, except that 9 equivalents of lithium hydroxide was added and the reaction mixture was stirred overnight. The resulting carboxylic acid was treated as described in step (ii), Method B above, but was further purified by preparative HPLC.

¹H NMR (500 MHz, CDCl₃) δ 1.49 (s, 9H), 1.52 (s, 9H), 2.00 (s, 3H), 2.42 (t, 2H), 3.14 (t, 2H), 3.35 (s, 2H), 3.48-3.56 (m, 4H), 4.10 (t, 2H), 7.19-7.26 (m, 3H), 7.29-7.33 (m, 2H), 7.53 (t, 1H), 7.83 (s, 1H), 9.10 (s, 1H)

MS m/z 589 (M+H)⁺

### (xix) tert-Butyl (6-methyl-5-{[({4-methyl-2-oxo-1-[(2-phenylethyl)amino]-1,2,5,6-tetrahydropyridin-3-yl}acetyl)amino]methyl}pyridin-2-yl)carbamate

The compound was prepared according to the same procedure as described with respect to Example 1(xviii) above.

¹H NMR (500 MHz, CDCl₃) δ 1.53 (s, 9H), 2.06 (s, 3H), 2.38 (s, 3H), 2.44 (t, 2H), 2.80 (t, 2H), 3.09 (t, 2H), 3.32 (s, 2H), 3.50 (t, 2H), 4.31 (d, 2H), 7.14 (s, 1H), 7.19-7.25 (m, 3H), 7.27-7.33 (m, 2H), 7.43 (d, 1H), 7.67 (d, 1H)

MS m/z 508 (M+H)⁺

### (xx) tert-Butyl {2-[({[1-(benzylamino)-4-methyl-2-oxo-1,2,5,6-tetrahydropyridin-3-yl]acetyl}amino)methyl]-4-chlorobenzyl}carbamate

The ester was hydrolysed according to step (i) above, except that 2 equivalents of lithium hydroxide was added and the reaction mixture was stirred for 64 hours.

¹H NMR (500 MHz, CDCl₃) δ 1.44 (s, 9H), 2.02 (s, 3H), 2.34 (t, 2H), 3.32 (s, 2H), 3.35 (t, 2H), 3.93-3.97 (m, 2H), 4.28 (d, 2H), 4.37 (d, 2H), 5.22 (br s, 1H), 5.55 (br s, 1H), 7.12-7.42 (m, 9H)

### (xxi) Di-tert-butyl ((E)-{[2-({[1-(benzylamino)-4-methyl-2-oxo-1,2,5,6-tetrahydropyridin-3-yl]acetyl}amino)ethoxy]amino}methylylidene)-biscarbamate

The ester was hydrolysed according to step (i) above, except that 2 equivalents of lithium hydroxide was added and the reaction mixture was stirred for 64 hours.

¹H NMR (500 MHz, CDCl₃) δ 1.48 (s, 18H), 1.97 (s, 3H), 2.29 (t, 2H), 3.30-3.36 (m, 4H), 3.51 (q, 2H), 3.97 (s, 2H), 4.09 (t, 2H), 5.60 (br s, 1H), 7.27-7.40 (m, 4H), 7.49 (t, 1H), 7.87 (s, 1H), 9.10 (s, 1H)

### (xxii) tert-Butyl {5-[({[1-(benzylamino)-4-methyl-2-oxo-1,2,5,6-tetrahydropyridin-3-yl]acetyl}amino)methyl]-6-methylpyridin-2-yl}carbamate

The ester was hydrolysed according to step (i) above, except that 2 equivalents of lithium hydroxide was added and the reaction mixture was stirred for 64 hours.

¹H NMR (500 MHz, CDCl₃) δ 1.50 (s, 9H), 2.02 (s, 3H), 2.29 (t, 2H), 2.39 (s, 3H), 3.30 (s, 2H), 3.33 (t, 2H) 3.91 (s, 2H), 4.31 (d, 2H), 5.50 (br s, 1H), 7.11 (br s, 1H), 7.21 (t, 1H), 7.26-7.34 (m, 5H), 7.43 (d, 1H), 7.68 (d, 1H)

### (xxiii) 2-[1-(Benzylamino)-4-methyl-2-oxo-1,2,5,6-tetrahydropyridin-3-yl]-N-[2-(1H-tetrazol-1-yl)benzyl]acetamide

The ester was hydrolysed according to step (i) above, except that 2 equivalents of lithium hydroxide was added and the reaction mixture was stirred for 48 hours. The crude carboxylic acid was dissolved in DMF and the specific amine (1.5 eq.), HOAT (1.5 eq.), EDC (2 eq.) and TEA (3 eq.) were added. The reaction mixture was stirred overnight at room temperature. The solution was diluted with water and extracted with DCM. The organic layers were washed with water, dried through a phase separator and then concentrated. The residue was chromatographed (SiO₂, DCM:MeOH, 97:3) to yield the title compound.

¹H NMR (500 MHz, CDCl₃) δ 1.96 (s, 3H), 2.33 (t, 2H), 3.25 (s, 2H), 3.36 (t, 2H), 3.97 (s, 2H), 4.19 (d, 2H), 5.55 (br s, 1H), 7.27-7.59 (m, 10H), 8.98 (s, 1H)

MS m/z 432 (M+H)⁺

### (xxiv) 2-[1-(Benzylamino)-4-methyl-2-oxo-1,2,5,6-tetrahydropyridin-3-yl]-N-(2-fluorobenzyl)acetamide

Ester hydrolysis was performed as described in Example 1(xxiii) above, except that 2.7 equivalents of lithium hydroxide was added in two portions and the reaction mixture was stirred for 43 hours. Amide coupling was then performed as described in Example 1(xxiii) above, after which the residue was purified by chromatography (SiO₂, heptane:ethyl acetate, 1:3) to give the title compound.

¹H NMR (500 MHz, CDCl₃) δ 2.01 (s, 3H), 2.28 (t, 2H), 3.30-3.34 (m, 4H), 3.94 (s, 2H), 4.43 (d, 2H), 5.52 (br s, 1H), 7.01 (t, 1H), 7.08 (t, 1H), 7.19-7.37 (m, 7H)

MS m/z 382 (M+H)⁺

### (xxv) tert-Butyl (4-chloro-2-{[({4-methyl-2-oxo-1-[(2-phenylethyl)amino]-1,2,5,6-tetrahydropyridin-3-yl}acetyl)amino]methyl}benzyl)carbamate

Amide coupling was performed as described in step (ii) method B above.

¹H NMR (500 MHz, CDCl₃) δ 1.46 (t, 9H), 2.06 (s, 3H), 2.47 (t, 2H), 2.79 (t, 2H), 3.11 (t, 2H), 3.33 (s, 2H), 3.52 (t, 2H), 4.27 (d, 2H), 4.38 (d, 2H), 5.22 (br s, 1H), 5.38 (br s, 1H), 7.13-7.17 (m, 2H), 7.18-7.24 (m, 4H), 7.27-7.32 (m, 2H), 7.42(br s, 1H)

MS m/z 541 (M+H)⁺

### (xxvi) tert-Butyl [5-({[(1-{[(2-methoxypyridin-3-yl)methyl]amino}-4-methyl-2-oxo-1,2,5,6-tetrahydropyridin-3-yl)acetyl]amino}methyl)-4,6-dimethylpyridin-2-yl]carbamate

The specific ester was hydrolysed with aqueous lithium hydroxide (1 M, 1.5 equiv.), stirring at room temperature overnight in THF:MeOH (1:1).

¹H NMR (500 MHz, CDCl₃) δ 1.52 (s, 9H), 2.05 (s, 3H), 2.34 (s, 3H), 2.38 (t, 2H), 2.45 (s, 3H), 3.25 (s, 2H), 3.42 (t, 2H), 3.90 (s, 2H), 4.00 (s, 3H), 4.34 (d, 2H), 5.71 (br s, 1H), 6.84 (dd, 1H), 6.98 (br s, 1H), 7.06 (s, 1H), 7.46 (dd, 1H), 7.60 (s, 1H), 8.11 (dd, 1H)

MS m/z 539 (M+H)⁺

### (xxvii) 2-[4-Chloro-2-({[(1-{[(2-methoxypyridin-3-yl)methyl]amino}-4-methyl-2-oxo-1,2,5,6-tetrahydropyridin-3-yl)acetyl]amino}methyl)-phenoxy]-N-ethylacetamide

¹H NMR (500 MHz, CD₃OD) δ 1.24 (t, 3H), 2.02 (s, 3H), 3.23 (s, 2H), 3.38-3.48 (m, 4H) 3.92 (d, 2H), 4.01 (s, 3H), 4.37(d, 2H), 4.46 (s, 2H), 5.82 (br s, 1H), 6.75 (d, 1H), 6.80 (dd, 1H), 7.19-7.26 (m, 3H), 7.45 (dd, 1H), 7.73 (br s, 1H), 8.10 (dd, 1H)

MS m/z 532 (M+H)⁺

### (xxvi) tert-Butyl (4-chloro-2-{[({1-[(2,2-difluoro-2-pyridin-2-ylethyl)-amino]-4-methyl-2-oxo-1,2,5,6-tetrahydropyridin-3-yl}acetyl)amino]-methyl}benzyl)carbamate

¹H NMR (500 MHz, CDCl₃) δ 8.67 (d, 1H), 7.81 (dt, 1H), 7.66 (d, 1H), δ 7.39 (dd, 1H), 7.31 (bs, 1H), 1.23 (d, 1H), 7.13-7.20 (m, 2H), 5.55 (t, 1H), 5.29 (bs, 1H), 4.37 (d, 2H), 4.27 d, 2H), 3.68-3.79 (m, 2H), 3.48 (t, 2H), 3.29 (s, 2H), 2.43 (t, 2H), 2.03 (s, 3H), 1.44 (s, 9H)

### (xxix) tert-Butyl (5-{[({1-[(2,2-difluoro-2-pyridin-2-ylethyl)amino]-4-methyl-2-oxo-1,2,5,6-tetrahydropyridin-3-yl}acetyl)amino]methyl}-4,6-dimethylpyridin-2-yl)carbamate

¹H NMR (500 MHz, CDCl₃) δ 8.67 (d, 1H), 7.83 (t, 1H), 7.66 (d, 1H), 7.58 (s, 1H), 7.37-7.42 (m, 1H), 7.12 (d, 1H), 6.84 (t, 1H), 5.50 (t, 1H), 4.34 (d, 2H), 3.63-3.74 (m, 2H), 3.44 (t, 2H), 3.24 (s, 2H), 2.37-2.44 (m, 5H), 2.32 (s, 3H), 2.04 (s, 3H), 1.52 (s, 9H)

### (xxx) N-[5-Chloro-2-(1H-tetrazol-1-yl)benzyl]-2-(1-{[(2-methoxypyridin-3-yl)methyl]amino}-4-methyl-2-oxo-1,2,5,6-tetrahydropyridin-3-yl)acetamide

¹H NMR (500 MHz, CD₃OD) δ 9.54 (s, 1H), 8.00 (dd, 1H), 7.60-7.65 (m, 2H), 7.48 (d, 1H), 7.53 (dd, 1H), 6.84 (dd, 1H), 4.16 (s, 2H), 3.93-3.99 (m, 5H), 3.48 (t, 2H), 3.22 (s, 2H), 2.46 (t, 2H), 1.91 (s, 3H)

MS m/z 499 (M+H)⁺

### (xxxi) N-[5-Chloro-2-(1H-tetrazol-1-yl)benzyl]-2-{1-[(2,2-difluoro-2-pyridin-2-ylethyl)amino]-4-methyl-2-oxo-1,2,5,6-tetrahydropyridin-3-yl}-acetamide

¹H NMR (500 MHz, CDCl₃) δ 9.02 (s, 1H), 8.67 (d, 1H), 7.83 (dt, 1H), 7.68 (d, 1H), 7.50 (d, 1H), 7.38-7.45 (m, 3H), 7.26 (d, 1H), 5.58 (t, 1H), 4.16 (d, 2H), 3.71-3.82 (m, 2H), 3.49 (t, 2H), 3.25 (s, 2H), 2.45 (t, 2H), 2.00 (s, 3H)

MS m/z 518 (M+H)⁺

### (xxxii) tert-Butyl [2-(4-chloro-2-{[({1-[(3-methoxybenzyl)amino]-4-methyl-2-oxo-1,2,5,6-tetrahydropyridin-3-yl}acetyl)amino]methyl}phenyl)-ethyl]carbamate

¹H NMR (500 MHz, CDCl₃) δ 1.41 (s, 9H), 2.01 (s, 3H), 2.34 (t, 2H), 2.79 (t, 2H), 3.21-3.32 (m, 4H), 3.37 (t, 2H), 3.79 (s, 3H), 3.91 (s, 2H), 4.37 (d, 2H), 5.00 (br s, 1H), 6.80 (d, 1H), 6.91 (d, 2H), 7.09 (d, 1H), 7.14 (s, 2H), 7.22 (t, 1H), 7.39 (br s, 1H)

MS m/z 571.27 (M+H)⁺

### (xxxiii) tert-Butyl (4-chloro-2-{[({1-[(3-methoxybenzyl)amino]-4-methyl-2-oxo-1,2,5,6-tetrahydropyridin-3-yl}acetyl)amino]methyl}benzyl)-carbamate

¹H NMR (500 MHz, CDCl₃) δ 1.44 (s, 9H), 2.02 (s, 3H), 2.36 (t, 2H), 3.32 (s, 2H), 3.38 (t, 2H), 3.80 (s, 3H), 3.94 (s, 2H), 4.27 (d, 2H), 4.37 (d, 2H), 5.00 (br s, 1H), 6.81 (d, 1H), 6.91 (d, 2H), 7.14 (s, 1H), 7.17-7.25 (m, 4H), 7.42 (br s, 1H)

MS m/z 557.22 (M+H)⁺

### (xxxiv) 2-(1-{[(6-Chloro-1,3-benzodioxol-5-yl)methyl]amino}-4-methyl-2-oxo-1,2,5,6-tetrahydropyridin-3-yl)-N-{5-chloro-2-[2-(ethylamino)-2-oxo-ethoxy]benzyl}acetamide

¹H NMR (500 MHz, CDCl₃) δ 1.24 (t, 3H), 2.02 (s, 3H), 2.38 (t, 2H), 3.27 (s, 2H), 3.37-3.45 (m, 4H), 3.96 (s, 2H), 4.40 (d, 2H), 4.46 (s, 2H), 5.98 (s, 2H), 6.74 (d, 1H), 6.83 (s, 1H), 6.84 (s, 1H), 7.18 (s, 1H), 7.21 (d, 1H), 7.28 (br s, 1H), 7.74 (br s, 1H)

MS m/z 577.06 (M+H)⁺

### (xxxv) tert-Butyl [4-chloro-2-({[(1-{[(6-chloro-1,3-benzodioxol-5-yl)-methyl]amino}-4-methyl-2-oxo-1,2,5,6-tetrahydropyridin-3-yl)acetyl]-amino}methyl)benzyl]carbamate

¹H NMR (500 MHz, CDCl₃) δ 1.44 (s, 9H), 2.03 (s, 3H), 2.43 (t, 2H), 3.31 (s, 2H), 3.45 (t, 2H), 4.00 (s, 2H), 4.28 (d, 2H), 4.35 (d, 2H), 5.30 (br s, 1H), 5.93 (s, 2H), 6.79 (s, 1H), 6.87 (s, 1H), 7.11 (s, 1H), 7.18 (d, 1H), 7.25 (d, 1H), 7.36 (br s, 1H)

MS m/z 605.47 (M+H)⁺

### (xxxvi) tert-Butyl [4-chloro-2-({[(1-{[(5-chloro-1,3-dimethyl-1H-pyrazol-4-yl)methyl]amino}-4-methyl-2-oxo-1,2,5,6-tetrahydropyridin-3-yl)acetyl]-amino}methyl)benzyl]carbamate

¹H NMR (500 MHz, CDCl₃) δ 1.45 (s, 9H), 2.04 (s, 3H), 2.25 (s, 3H), 2.40 (t, 2H), 2.97 (s, 2H), 3.33 (s, 2H), 3.41 (t, 2H), 3.74 (s, 3H), 3.80 (d, 2H), 4.38 (d, 2H), 7.14-7.29 (m, 3H), 7.37 (br. s, 1H), 8.02 (br. s, 1H)

### (xxxvii) tert-Butyl [5-({[(1-{[(5-chloro-1,3-dimethyl-1H-pyrazol-4-yl)-methyl]amino}-4-methyl-2-oxo-1,2,5,6-tetrahydropyridin-3-yl)acetyl]-amino}methyl)-4,6-dimethylpyridin-2-yl]carbamate

1H NMR (500 MHz, CDCl₃) δ 1.52 (s, 9H), 1.53 (s, 6H), 2.05 (s, 3H), 2.25 (s, 3H), 2.45 (t, 2H), 3.27 (s, 2H), 3.37 (t, 2H), 3.73 (d, 2H), 3.77 (s, 3H), 4.35 (d, 2H), 7.10 (br. s, 1H), 7.59 (br. s, 1H), 7.63 (br. s, 1H)

### (xxxviii) 2-[4-Chloro-2-({[(1-{[(5-chloro-1,3-dimethyl-1H-pyrazol-4-yl)-methyl]amino}-4-methyl-2-oxo-1,2,5,6-tetrahydropyridin-3-yl)acetyl]-amino}methyl)phenoxy]-N-ethylacetamide

¹H NMR (500 MHz, CDCl₃) δ 1.22 (t, 3H), 2.00 (s, 3H), 2.22 (s, 3H), 2.34 (t, 2H), 3.25 (s, 2H), 3.32-3.45 (m, 4H), 3.74 (s, 5H), 4.40 (d, 2H), 4.43 (s, 2H), 5.26 (br s, 1H), 6.72 (d, 1H), 7.16-7.28 (m, 2H), 7.74 (br s, 1H)

MS m/z 551 (M+H)⁺

### (xxxix) tert-Butyl (4-chloro-2-{[({4-methyl-2-oxo-1-[(pyridin-3-ylmethyl)-amino]-1,2,5,6-tetrahydropyridin-3-yl}acetyl)amino]methyl}benzyl)-carbamate

MS m/z 528 (M+H)⁺

### (xl) tert-Butyl (4,6-dimethyl-5-{[({4-methyl-2-oxo-1-[(pyridin-3-ylmethyl)-amino]-1,2,5,6-tetrahydropyridin-3-yl}acetyl)amino]methyl}pyridin-2-yl)-carbamate

MS m/z 509 (M+H)⁺

### (xli) 2-(1-{[(5-Chloro-1,3-dimethyl-1H-pyrazol-4-yl)methyl]amino}-4-methyl-2-oxo-1,2,5,6-tetrahydropyridin-3-yl)-N-[5-chloro-2-(1H-tetrazol-1-yl)benzyl]acetamide

The compound was purified by prep-HPLC.

¹H NMR (500 MHz, CDCl₃) δ 2.01 (s, 3H), 2.27 (s, 3H), 2.42 (t, 2H), 3.29 (s, 2H), 3.44 (t, 2H), 3.76 (s, 3H), 3.85 (s, 2H), 4.16 (d, 2H), 5.36 (br. s, 1H), 7.26-7.30 (m, 2H), 7.41-7.46 (m, 1H), 7.48-7.54 (m, 2H)

MS m/z 519 (M+H)⁺

### (xlii) 2-(1-{[(5-Chloro-1,3-dimethyl-1H-pyrazol-4-yl)methyl]amino}-4-methyl-2-oxo-1,2,5,6-tetrahydropyridin-3-yl)-N-[5-fluoro-2-(hydroxymethyl)benzyl]acetamide

¹H NMR (500 MHz, CDCl₃) δ 2.02 (s, 3H), 2.25 (s, 3H), 2.38 (t, 2H), 3.28 (s, 2H), 3.41 (t, 2H), 3.71 (br. s, 1H), 3.75 (s, 3H), 3.80 (s, 2H), 4.43 (d, 2H), 4.66 (s, 2H), 5.38 (br. s, 1H), 6.91-7.01 (m, 2H), 7.31-7.36 (m, 1H), 7.39-7.45 (m, 1H)

MS m/z 465 (M+H)⁺

### (xliii) 2-(1-{[(5-Chloro-1,3-dimethyl-1H-pyrazol-4-yl)methyl]amino}-4-methyl-2-oxo-1,2,5,6-tetrahydropyridin-3-yl)-N-[5-chloro-2-(hydroxymethyl)benzyl]acetamide

¹H NMR (500 MHz, CDCl₃) δ 2.02 (s, 3H), 2.25 (s, 3H), 2.39 (t, 2H), 3.28 (s, 2H), 3.41 (t, 2H), 3.75 (s, 3H), 3.81 (s, 2H), 4.41 (d, 2H), 4.66 (s, 2H), 7.21-7.25 (m, 2H), 7.29-7.33 (m, 1H), 7.37-7.43 (m, 1H)

MS m/z 481 (M+H)⁺

### (xliv) 2-(4-Chloro-2-{[({4-methyl-2-oxo-1-[(pyridin-3-ylmethyl)amino]-1,2,5,6-tetrahydropyridin-3-yl}acetyl)amino]methyl}phenoxy)-N-ethylacetamide

¹H NMR (500 MHz, CDCl₃) δ 1.25 (t, 3H), 2.01 (s, 3H), 2.33 (t, 2H), 3.27 (s, 2H), 3.34 (t, 2H), 3.42 (p, 2H), 3.94 (s, 2H), 4.44 (d, 2H), 4.47 (s, 2H), 6.76 (d, 1H), 7.14-7.25 (m, 3H), 7.26-7.33 (m, 2H), 7.66-7.72 (m, 1H), 7.75 (br. s, 1H), 8.53-8.58 (m, 2H)

MS m/z 500 (M+H)⁺

### (xlv) N-[5-Chloro-2-(1H-tetrazol-1-yl)benzyl]-2-{4-methyl-2-oxo-1-[(pyridin-3-ylmethyl)amino]-1,2,5,6-tetrahydropyridin-3-yl}acetamide

¹H NMR (500 MHz, CDCl₃) δ 2.00 (s, 3H), 2.39 (t, 2H), 3.28 (s, 2H), 3.40 (t, 2H), 4.05 (s, 2H), 4.17 (d, 2H), 5.57 (br. s, 1H), 7.26-7.31 (m, 2H), 7.37-7.43 (m, 2H), 7.43-7.47 (m, 1H), 7.51-7.54 (m, 1H), 7.73-7.79 (m, 1H), 8.51-8.56 (m, 1H), 8.62 (br. s, 1H)

MS m/z 467 (M+H)⁺

### (xlvi) 2-[1-(Benzylamino)-4-methyl-2-oxo-1,2,5,6-tetrahydropyridin-3-yl]-N-(5-chloro-2-methylbenzyl)acetamide

The specific ester was hydrolysed as described in step (i) and the amide coupling was performed as in step (ii), Method B.

¹H NMR (500 MHz, CD₃OD) δ 1.96 (s, 3H), 2.30 (s, 3H), 2.38 (t, 2H), 3.38 (s, 2H), 3.39 (t, 2H), 3.98 (s, 2H), 4.34 (s, 2H), 4.87 (s, 2H), 7.13-7.41 (m, 8H)

HRMS (ESI) calculated for C₂₃H₂₆ClN₃O₂ 412.1792 (M+H)⁺, found 412.1786.

### (xlvii) 2-(1-{[(5-Chloro-1,3-dimethyl-1H-pyrazol-4-yl)methyl]amino}-4-methyl-2-oxo-1,2,5,6-tetrahydropyridin-3-yl)-N-(5-chloro-2-hydroxybenzyl)acetamide

¹H NMR-(500 MHz, CDCl₃) δ 2.07 (s, 3H), 2.26 (s, 3H), 2.39 (t, 2H), 3.35 (s, 2H), 3.41 (t, 2H), 3.76 (s, 3H), 3.81 (s, 2H), 4.38 (d, 2H), 7.11-7.14 (m, 1H), 7.19-7.21 (m, 1H), 7.19-7.21 (m, 1H), 7.21-7.25 (m, 1H), 7.34 (br s, 1H)

MS m/z 446 (M+H)⁺

### (xlviii) N-(3-Chlorobenzyl)-2-(1-{[(5-chloro-1,3-dimethyl-1H-pyrazol-4-yl)methyl]amino}-4-methyl-2-oxo-1,2,5,6-tetrahydropyridin-3-yl)acetamide

¹H NMR (500 MHz, CDCl₃) δ 2.01 (s, 3H), 2.26 (s, 3H), 2.33 (t, 2H), 3.30 (s, 2H), 3.37 (t, 2H), 3.78 (s, 3H), 3.83 (s, 2H), 4.21 (d, 2H), 6.86 (d, 1H), 7.08 (d, 1H), 7.12-7.16 (m, 1H), 7.94 (br s, 1H)

MS m/z 450(M+H)⁺

### (xlix) tert-Butyl [4-chloro-2-({[(1-{[(5-chloro-2-thienyl)methyl]amino}-4-methyl-2-oxo-1,2,5,6-tetrahydropyridin-3-yl)acetyl]amino}methyl)benzyl]-carbamate

MS m/z 567 (M+H)⁺

### (l) tert-Butyl [2-({[(1-{[(5-chloro-2-thienyl)methyl]amino}-4-methyl-2-oxo-1,2,5,6-tetrahydropyridin-3-yl)acetyl]amino}methyl)-4-methylbenzyl]-carbamate

MS m/z 547 (M+H)⁺

### Example 2

Unless otherwise stated, the compounds (i) to (ix) listed below were prepared from the corresponding compounds of Example 1 by Method A below. The compounds (x) to (xxxiv) listed below were prepared from the corresponding compounds of Example 1 by Method B below.

Method A

Palladium on carbon (10%, 1 mass equiv.) and HCl (conc., 2-3 drops) were added to a solution of the specific benzyloxycarbonyl-protected compound is (0.03 mmol; see Example 1 above) in methanol (3 mL). The suspension was hydrogenated under atmospheric pressure at room temperature for 90 minutes. The suspension was filtered through Celite®, washed with methanol (3×5 mL) and the solvent was removed under reduced pressure. The residue was dissolved in a minimum volume of methanol and the deprotected product was precipitated from ethyl acetate. Yields were nearly quantitative.

Method B

The specific amide (0.04 mmol; see Example 1 above) was dissolved in ethyl acetate saturated with HCl (2 mL), and stirred at room temperature for 2 hours. The solvent and excess of reagents were evaporated under reduced pressure and to give the product.

### (i) N-(4-Carbamimidoylbenzyl)-2-(4-methyl-2-oxo-1-phenylmethanesulfonylamino-1,2,5,6-tetrahydropyridin-3-yl)acetamide

¹H NMR (400 MHz, CD₃OD) δ 2.06 (s, 3H), 2.55 (br t, 2H), 3.36 (s, 2H), 3.58 (br t, 2H), 4.28 (s, 2H), 4.41 (s, 2H), 7.06-7.31 (m, 5H), 7.45 (d, 2H), 7.58 (d, 2H)

MS m/z 470.4 (M+H)⁺

### (ii) N-(4-Carbamimidoylbenzyl)-2-[1-(3-methoxybenzenesulfonylamino)-4-methyl-2-oxo-1,2,5,6-tetrahydropyridin-3-yl]acetamide

¹H NMR (400 MHz, CD₃OD) δ 1.96 (s, 3H), 2.59 (t, 2H), 3.22 (s, 2H), 3.68 (t, 2H), 3.82 (s, 3H), 4.40 (s, 2H), 7.12 (dd, 1H), 7.36-7.48 (m, 5H), 7.76 (d, 2H), 8.76 (br s, 2H), 9.23 (br s, 2H)

MS m/z 484.0 (M+H)⁺

### (iii) N-(4-Carbamimidoylbenzyl)-2-[4-methyl-1-(naphthalene-1-sulfonylamino)-2-oxo-1,2,5,6-tetrahydropyridin-3-yl]acetamide

¹H NMR (400 MHz, CD₃OD) δ 1.90 (s, 3H), 2.47 (s, 2H), 3.09 (s, 2H), 3.50 (s, 2H), 4.33 (s, 2H), 7.41 (d, 2H), 7.55 (d, 1H), 7.54-7.63 (m, 1H), 7.68 (d, 1H), 7.72 (d, 2H), 7.97 (d, 1H), 8.14 (d, 1H), 8.26 (d, 1H), 8.74 (br s, 2H), 8.82 (d, 1H), 9.23 (br s, 2H)

MS m/z 506.4 (M+H)⁺

### (iv) N-(4-Carbamimidoylbenzyl)-2-[1-(2,5-dimethylbenzenesulfonylamino)-4-methyl-2-oxo-1,2,5,6-tetrahydropyridin-3-yl]acetamide

¹H NMR (400 MHz, CD₃OD) δ 1.93 (s, 3H), 2.32 (s, 3H), 2.50 (t, 2H), 2.69 (s, 3H), 3.21 (s, 2H), 3.58 (t, 2H), 4.39 (s, 2H), 7.20 (d, 1H), 7.29 (d, 1H), 7.48 (d, 2H), 7.76 (m, 3H), 8.75 (br s, 2H), 9.24 (br s, 2H)

MS m/z 484.5 (M+H)⁺

### (v) N-(4-Carbamimidoylbenzyl)-2-[1-(2,5-dichlorobenzenesulfonylamino)-4-methyl-2-oxo-1,2,5,6-tetrahydropyridin-3-yl]acetamide

¹H NMR (400 MHz, CD₃OD) δ 1.94 (s, 3H), 2.59 (t, 2H), 3.23 (s, 2H), 3.72 (t, 2H), 4.40 (s, 2H), 7.48 (d, 2H), 7.55 (s, 2H), 7.77 (d, 2H), 7.97 (s, 1H), 8.77 (br s, 2H), 9.24 (br s, 2H)

MS m/z 524.1 (M+H)⁺

### (vi) N-(4-Carbamimidoylbenzyl)-2-[1-(2-methoxy-4-methylbenzenesulfonylamino)-4-methyl-2-oxo-1,2,5,6-tetrahydropyridin-3-yl]acetamide

¹H NMR (400 MHz, CD₃OD) δ 1.91 (s, 3H), 2.36 (s, 3H), 2.51 (s, 2H), 3.22 (s, 2H), 3.69 (s, 2H), 4.01 (s, 3H), 4.38 (s, 2H), 6.79 (d, 2H), 6.99 (s, 1H), 7.46 (d, 2H), 7.65 (d, 1H), 7.77 (d, 2H), 8.78 (br s, 2H), 9.26 (br s, 2H)

MS m/z 500.4 (M+H)⁺.

### (vii) N-(4-Carbamimidoylbenzyl)-2-[1-(2-chloro-6-methylbenzenesulfonylamino)-4-methyl-2-oxo-1,2,5,6-tetrahydropyridin-3-yl]acetamide

¹H NMR (400 MHz, CD₃OD) δ 1.95 (s, 3H), 2.59 (t, 2H), 2.68 (s, 3H), 3.22 (s, 2H), 3.72 (t, 2H), 4.39 (s, 2H), 7.22 (d, 2H), 7.32-7.41 (m, 2H), 7.48 (d, 2H), 7.78 (d, 2H), 8.78 (br s, 2H), 9.25 (br s, 2H)

MS m/z 504.2 (M+H)⁺

### (viii) N-(4-Carbamimidoylbenzyl)-2-[1-(4-chloro-2-fluorobenzenesulfonylamino)-4-methyl-2-oxo-1,2,5,6-tetrahydropyridin-3-yl]acetamide

¹H NMR (400 MHz, CD₃OD) δ 1.94 (s, 3H), 2.60 (s, 2H), 3.20 (s, 2H), 3.74 (s, 2H), 4.39 (s, 2H), 7.27 (d, 1H), 7.34 (d, 1H), 7.48 (d, 2H), 7.77 (d, 2H), 7.81-7.83 (m 1H), 8.76 (br s, 2H), 9.24 (br s, 2H)

MS m/z 508.2 (M+H)⁺

### (ix) 2-(1-Benzenesulfonylamino-4-methyl-2-oxo-1,2,5,6-tetrahydropyridin-3-yl)-N-(4-carbamimidoylbenzyl)acetamide

¹H NMR (400 MHz, CD₃OD) δ 1.95 (s, 3H), 2.59 (s, 2H), 3.19 (s, 2H), 3.68 (t, 2H), 4.39 (s, 2H), 7.46-7.50 (m, 4H), 7.56-7.58 (m, 1H), 7.76 (d, 2H), 7.90 (d, 2H), 8.76 (br s, 2H), 9.24 (br s, 2H)

MS m/z 456.4 (M+H)⁺

### (x) N-[2-(Aminomethyl)-5-chlorobenzyl]-2-{1-[(benzylsulfonyl)amino]-4-methyl-2-oxo-1,2,5,6-tetrahydropyridin-3-yl}acetamide hydrochloride

The reaction mixture was stirred for 5 hours.

¹H NMR (500 MHz, CD₃OD) δ 1.96 (s, 3H), 2.58 (t, 2H), 3.36 (s, 2H), 3.66 (t, 2H), 4.22 (s, 2H), 4.31 (s, 2H), 4.40 (s, 2H), 7.24 (dd, 1H), 7.32-7.43 (m, 6H), 7.46 (d, 1H)

MS m/z 491.1 (M+H)⁺

### (xi) N-[(6-Amino-2-methylpyridin-3-yl)methyl]-2-{1-[(benzylsulfonyl)-amino]-4-methyl-2-oxo-1,2,5,6-tetrahydropyridin-3-yl}acetamide hydrochloride

The reaction mixture was stirred for 8 hours.

¹H NMR (500 MHz, CD₃OD) δ 1.97 (s, 3H), 2.47 (s, 3H), 2.58 (t, 2H), 3.35 (s, 2H), 3.68 (t, 2H), 4.23 (s, 2H), 4.32 (s, 2H), 6.74 (d, 1H), 7.32-7.37 (m, 3H), 7.38-7.43 (m, 2H), 7.84 (d, 1H)

MS m/z 458.1 (M+H)⁺

### (xii) N-[(6-Amino-2-methylpyridin-3-yl)methyl]-2-{4-methyl-1-[(1-naphthylsulfonyl)amino]-2-oxo-1,2,5,6-tetrahydropyridin-3-yl}acetamide hydrochloride

¹H NMR (500 MHz, CD₃OD) δ 8.81 (d, 1H), 8.26 (d, 1H), 8.18 (d, 1H), 8.00 (d, 1H), 7.55-7.77 (m, 4H), 6.80 (d, 1H), 4.13 (s, 2H), 3.43 (t, 2H), 3.05 (s, 2H), 2.38-2.52 (m, 5H), 1.90 (s, 3H)

MS m/z 494 (M+H)⁺

### (xiii) N-[2-(Aminomethyl)benzyl]-2-{4-methyl-1-[(1-naphthylsulfonyl)-amino]-2-oxo-1,2,5,6-tetrahydropyridin-3-yl}acetamide hydrochloride

¹H NMR (500 MHz, CD₃OD) δ 8.82 (d, 1H), 8.28 (d, 1H), 8.19 (d, 1H), 8.01 (d, 1H), 7.71 (t, 1H), 7.65 (t, 1H), 7.58 (t, 1H), 7.30-7.45 (m, 4H), 4.31 (s, 2H), 4.19 (s, 2H), 3.41 (t, 2H), 3.05 (s, 2H), 2.41 (t, 2H), 1.86 (s, 3H)

MS m/z 594 (M+H)⁺

### (xiv) N-[2-(Aminomethyl)-5-chlorobenzyl]-2-{4-methyl-2-oxo-1-[(phenylsulfonyl)amino]-1,2,5,6-tetrahydropyridin-3-yl}acetamide hydrochloride

The reaction mixture was stirred for 6 hours.

¹H NMR (500 MHz, CD₃OD) δ 1.91 (s, 3H), 2.56 (t, 2H), 3.13 (s, 2H), 3.63 (t, 2H), 4.20 (s, 2H), 4.31 (s, 2H), 7.34-7.43 (m, 3H), 7.50 (t, 2H), 7.60 (t, 1H), 7.90 (d, 2H)

MS m/z 477.03 (M+H)⁺

### (xv) N-[2-({[Amino(imino)methyl]amino}oxy)ethyl]-2-{4-methyl-2-oxo-1-[(phenylsulfonyl)amino]-1,2,5,6-tetrahydropyridin-3-yl}acetamide hydrochloride

The reaction mixture was stirred over night.

¹H NMR (500 MHz, CD₃OD) δ 1.95 (s, 3H), 2.58 (t, 2H), 3.11 (s, 2H), 3.38 (t, 2H), 3.65 (t, 2H), 3.86 (t, 2H), 7.52 (t, 2H), 7.62 (t, 1H), 7.89 (d, 2H)

MS m/z 425.30 (M+H)⁺

### (xvi) N-[(6-Amino-2-methylpyridin-3-yl)methyl]-2-{4-methyl-2-oxo-1-[(phenylsulfonyl)amino]-1,2,5,6-tetrahydropyridin-3-yl}acetamide hydrochloride

The reaction mixture was stirred overnight.

¹H NMR (500 MHz, CD₃OD) δ 1.92 (s, 3H), 2.46 (s, 3H), 2.55 (t, 2H), 3.10 (s, 2H), 3.62 (t, 2H), 4.15 (s, 2H), 6.79 (d, 1H), 7.50 (t, 2H), 7.59 (t, 1H), 7.73 (d, 1H), 7.88 (d, 2H)

MS m/z 444.07 (M+H)⁺

### (xvii) N-[2-({[Amino(imino)methyl]amino}oxy)ethyl]-2-{4-methyl-2-oxo-1-[(2-phenylethyl)amino]-1,2,5,6-tetrahydropyridin-3-yl}acetamide hydrochloride

¹H NMR (500 MHz, CD₃OD) δ 2.00 (s, 3H), 2.68 (t, 2H), 3.05 (t, 2H), 3.40 (s, 2H), 3.51 (t, 2H), 3.60 (t, 2H), 3.81 (t, 2H), 3.96 (t, 2H), 7.24-7.40 (m, 5H)

MS m/z 389 (M+H)⁺

### (xviii) N-[(6-Amino-2-methylpyridin-3-yl)methyl]-2-{4-methyl-2-oxo-1-[(2-phenylethyl)amino]-1,2,5,6-tetrahydropyridin-3-yl}acetamide hydrochloride

¹H NMR (500 MHz, CD₃OD) δ 1.99 (t, 3H), 2.54 (s, 3H), 2.66 (t, 2H), 3.03 (t, 2H), 3.39 (s, 2H), 3.60 (t, 2H), 3.81 (t, 2H), 4.28 (s, 2H), 6.85 (d, 1H), 7.25-7.37 (m, 5H), 7.88 (d, 1H)

MS m/z 408 (M+H)⁺

### (xix) N-[2-(Aminomethyl)-5-chlorobenzyl]-2-[1-(benzylamino)-4-methyl-2-oxo-1,2,5,6-tetrahydropyridin-3-yl]acetamide hydrochloride

The reaction mixture was stirred for 5 hours.

¹H NMR (400 MHz, CD₃OD) δ 1.94 (s, 3H), 2.51-2.59 (m, 2H), 3.37 (s, 2H), 3.55-3.64 (m, 2H), 4.26 (s, 2H), 4.29-4.37 (m, 2H), 4.41 (s, 2H), 7.35-7.52 (m, 8H)

MS m/z 427.1 (M+H)⁺

### (xx) N-[2-({[Amino(imino)methyl]amino}oxy)ethyl]-2-[1-(benzylamino)-4-methyl-2-oxo-1,2,5,6-tetrahydropyridin-3-yl]acetamide hydrochloride

The reaction mixture was stirred for 24 hours.

¹H NMR (400 MHz, CD₃OD) δ 1.98 (s, 3H), 2.64 (t, 2H), 3.36 (s, 2H), 3.49 (t, 2H), 3.66-3.73 (m, 2H), 3.95 (t, 2H), 4.46-4.50 (m, 2H), 7.45-7.50 (m, 3H), 7.51-7.56 (m, 2H)

MS m/z 375 (M+H)⁺

### (xxi) N-[(6-Amino-2-methylpyridin-3-yl)methyl]-2-[1-(benzylamino)-4-methyl-2-oxo-1,2,5,6-tetrahydropyridin-3-yl]acetamide hydrochloride

The reaction mixture was stirred for 96 hours.

¹H NMR (500 MHz, CD₃OD) δ 1.96 (s, 3H), 2.53 (s, 3H), 2.57-2.65 (m, 2H), 3.34 (s, 2H), 3.61-3.73 (m, 2H), 4.26 (s, 2H), 4.38-4.49 (m, 2H), 6.83 (d, 1H), 7.42-7.57 (m, 5H), 7.86 (d, 1H)

MS m/z 394 (M+H)⁺

### (xxii) N-[2-(Aminomethyl)-5-chlorobenzyl]-2-{4-methyl-2-oxo-1-[(2-phenylethyl)amino]-1,2,5,6-tetrahydropyridin-3-yl}acetamide hydrochloride

¹H NMR (500 MHz, CD₃OD) δ 1.95 (t, 3H), 2.62 (t, 2H), 3.00 (t, 2H), 3.03 3.39 (s, 2H), 3.54 (t, 2H), 3.76 (t, 2H), 4.24 (s, 2H), 4.41 (s, 2H), 7.22-7.28 (m, 3H), 7.28-7.35 (m, 3H), 7.39 (d, 1H), 7.47 (d, 1H)

MS m/z 441 (M+H)⁺

### (xxiii) N-[(6-Amino-2,4-dimethylpyridin-3-yl)methyl]-2-(1-{[(2-methoxypyridin-3-yl)methyl]amino}-4-methyl-2-oxo-1,2,5,6-tetrahydropyridin-3-yl)acetamide

After deprotection, the compound was purified by prep-HPLC.

¹H NMR (500 MHz, CDCl₃) δ 1.93 (s, 3H), 2.38 (s, 3H), 2.45 (t, 2H), 2.49 (s, 3H), 3.21 (s, 2H), 3.48 (t, 2H), 3.96 (s, 2H), 3.98 (s, 3H), 4.28 (s, 2H), 6.55 (s, 1H), 6.90 (dd, 1H), 7.63 (dd, 1H), 8.05 (dd, 1H)

MS m/z 439 (M+H)⁺

### (xxiv) N-[(6-Amino-2,4-dimethylpyridin-3-yl)methyl]-2-{1-[(2,2-difluoro-2-pyridin-2-ylethyl)amino]-4-methyl-2-oxo-1,2,5,6-tetrahydropyridin-3-yl}-acetamide acetate

The crude product was purified by Prep-HPLC.

¹H NMR (500 MHz, CD₃OD) δ 8.65 (d, 1H); 7.92 (dt, 1H), 7.74 (d, 1H), 7.52 (dd, 1H), 6.40 (s, 1H), 4.29 (2, 2H), 3.70 (t, 2H), 3.37 (t, 2H), 3.24 (s, 2H), 2.43 (s, 3H), 2.38 (t, 2H), 2.30 (s, 3H), 1.97 (s, 3H), 1.91 (s, 3H)

MS m/z 460 (M+H)⁺

### (xxv) N-[2-(Aminomethyl)-5-chlorobenzyl]-2-{1-[(2,2-difluoro-2-pyridin-2-ylethyl)amino]-4-methyl-2-oxo-1,2,5,6-tetrahydropyridin-3-yl}acetamide acetate

The crude product was purified by Prep-HPLC.

¹H NMR (500 MHz, CD₃OD) δ 8.65 (d, 1H), 7.96 (dt 1H), 7.74 (d, 1H), 7.52 (dd, 1H), 7.42 (d, 1H), 7.40 (d, 1H), 7.34 (dd, 1H), 4.40 (s, 2H), 4.15 (s, 2H), 3.71 (t, 2H), 3.38 (t, 2H), 3.29 (s, 2H), 2.40 (t, 2H), 1.92 (s, 3H), 1.91 (s, 3H)

MS m/z 481 (M+H)⁺

### (xxvi) N-[2-(2-Aminoethyl)-5-chlorobenzyl]-2-{1-[(3-methoxybenzyl)-amino]-4-methyl-2-oxo-1,2,5,6-tetrahydropyridin-3-yl}acetamide acetate

The compound was purified by prep-HPLC.

¹H NMR (500 MHz, CD₃OD) δ 1.93 (s, 3H), 1.94 (s, 3H), 2.39 (t, 2H), 3.01 (t, 2H), 3.17 (t, 2H), 3.35 (s, 2H), 3.41 (t, 2H), 3.79 (s, 3H), 3.96 (s, 2H), 4.40 (s, 2H), 6.84 (dd, 1H), 6.95 (d, 1H), 6.97 (s, 1H), 7.22-7.27 (m, 3H), 7.36 (s, 1H)

MS m/z 473.00 (M+H)⁺

### (xxvii) N-[2-(Aminomethyl)-5-chlorobenzyl]-2-{1-[(3-methoxybenzyl)-amino]-4-methyl-2-oxo-1,2,5,6-tetrahydropyridin-3-yl}acetamide acetate

The compound was purified by prep-HPLC.

¹H NMR (500 MHz, CD₃OD) δ 1.89 (s, 3H), 1.90 (s, 3H), 2.36 (t, 2H), 3.30 (s, 2H), 3.38 (t, 2H), 3.78 (s, 3H), 3.91 (s, 2H), 4.17 (s, 2H), 4.39 (s, 2H), 6.83 (dd, 1H), 6.92 (d, 2H), 6.94 (s, 1H), 7.22 (t, 1H), 7.33 (dd, 1H), 7.39 (d, 1H), 7.42 (s, 1H)

MS m/z 457.07 (M+H)⁺

### (xxviii) N-[2-(Aminomethyl)-5-chlorobenzyl]-2-(1-{[(6-chloro-1,3-benzodioxol-5-yl)methyl]amino}-4-methyl-2-oxo-1,2,5,6-tetrahydropyridin-3-yl)-acetamide hydrochloride

¹H NMR (500 MHz, CD₃OD) δ 1.96 (s, 3H), 2.56 (s, 2H), 3.33 (m, 2H), 3.60 (br s, 2H), 4.27 (s, 2H), 4.29 (br s, 2H), 4.42 (s, 2H), 6.03 (s, 2H), 6.95 (s, 1H), 7.06 (br s, 1H), 7.37 (d, 1H), 7.44 (d, 1H), 7.47 (s, 1H)

MS m/z 505.01 (M+H)⁺

### (xxix) N-[2-(Aminomethyl)-5-chlorobenzyl]-2-(1-{[(5-chloro-1,3-dimethyl-1H-pyrazol-4-yl)methyl]amino}-4-methyl-2-oxo-1,2,5,6-tetrahydropyridin-3-yl)acetamide

¹H NMR (500 MHz, CDCl₃) δ 2.03 (s, 3H), 2.27 (s, 3H), 2.40 (t, 2H), 3.32 (s, 2H), 3.42 (t, 2H), 3.76 (s, 3H), 3.80 (s, 2H), 3.91 (br s, 2H), 4.40 (d, 2H), 7.20-7.24 (m, 2H), 7.25-7.26 (m, 1H), 7.81 (br s, 1H)

MS m/z 479 (M+H)⁺

### (xxx) N-[(6-Amino-2,4-dimethylpyridin-3-yl)methyl]-2-(1-{[(5-chloro-1,3-dimethyl-1H-pyrazol-4-yl)methyl]amino}-4-methyl-2-oxo-1,2,5,6-tetrahydropyridin-3-yl)acetamide

¹H NMR (500 MHz, CDCl₃) δ 2.03 (s, 3H), 2.06 (s, 3H), 2.21-2.25 (m, 6H), 2.34 (t, 2H), 2.40 (s, 2H), 3.24 (s, 2H), 3.35 (t, 2H), 3.73 (s, 2H), 3.76 (s, 3H), 4.26 (d, 2H), 5.27 (br s, 1H), 6.18 (br s, 1H), 6.94 (br s, 1H)

MS m/z 460 (M+H)⁺

### (xxi) N-[(6-Amino-2,4-dimethylpyridin-3-yl)methyl]-2-{4-methyl-2-oxo-1-[(pyridin-3-ylmethyl)amino]-1,2,5,6-tetrahydropyridin-3-yl}acetamide

¹H NMR (500 MHz, CDCl₃) δ 2.04 (s, 3H), 2.26 (s, 3H), 2.32 (t, 2H), 2.42 (s, 3H), 3.26 (s, 2H), 3.33 (t, 2H), 3.92 (s, 2H), 4.30 (d, 2H), 4.99 (br. s, 1H), 5.44 (br. s, 1H), 6.22 (s, 1H), 6.82 (br. s, 1H), 7.26-7.32 (m, 1H), 7.67-7.72 (m, 1H), 8.55-8.59 (1H)

MS m/z 409 (M+H)⁺

### (xxxii) N-[2-(Aminomethyl)-5-chlorobenzyl]-2-{4-methyl-2-oxo-1-[(pyridin-3-ylmethyl)amino]-1,2,5,6-tetrahydropyridin-3-yl}acetamide

¹H NMR (500 MHz, CDCl₃) δ 2.01 (s, 3H), 2.36 (t, 2H), 3.30 (s, 2H), 3.37 (t, 2H), 3.93 (s, 2H), 3.98 (s, 2H), 4.42 (d, 2H), 7.20-7.31 (m, 4H), 7.68-7.74 (m, 1H), 7.89 (br. s, 1H), 8.52-8.56 (m, 1H), 8.59 (br. s, H)

MS m/z 428 (M+H)⁺

### (xxxiii) N-[2-(Aminomethyl)-5-chlorobenzyl]-2-(1-{[(5-chloro-2-thienyl)-methyl]amino}-4-methyl-2-oxo-1,2,5,6-tetrahydropyridin-3-yl)acetamide acetate

The compound was purified by prep-HPLC.

¹H NMR (500 MHz, CDCl₃) δ 2.03 (s, 3H), 2.04 (s, 3H), 2.41 (t, 2H), 3.31 (s, 2H), 3.44 (t, 2H), 3.92 (s, 2H), 4.07 (s, 2H), 4.42 (d, 2H), 6.71-6.76 (m, 2H), 7.21-7.27 (m, 3H), 7.80 (br s, 1H)

MS m/z 469 (M+H)⁺

### (xxxiv) N-[2-(Aminomethyl)-5-methylbenzyl]-2-(1-{[(5-chloro-2-thienyl)-methyl]amino}-4-methyl-2-oxo-1,2,5,6-tetrahydropyridin-3-yl)acetamide acetate

The compound was purified by prep-HPLC.

¹H NMR (500 MHz, CDCl₃) δ 1.95 (s, 3H), 1.98 (s, 3H), 2.32 (s, 3H), 2.38 (t, 2H), 3.27 (s, 2H), 3.40 (t, 2H), 3.97 (s, 2H), 4.03 (s, 2H), 4.40 (br s, 2H), 6.72 (d, 1H), 6.76 (d, 1H), 7.08 (d, 1H), 7.12 (br s, 1H), 7.23 (d, 1H), 8.27 (br s, 1H)

MS m/z 447 (M+H)⁺

### Example 3

### [4-Chloro-2-({2-[4-methyl-1-(naphthalene-1-sulfonylamino)-2-oxo-1,2,5,6-tetrahydropyridin-3-yl]acetylamino}methyl)benzyl]carbamic acid tert-butyl ester

[4-Methyl-1-(naphthalene-1-sulfonylamino)-2-oxo-1,2,5,6-tetrahydropyridin-3-yl]acetic acid ethyl ester (0.12 mmol; see Preparation 2(iii) above) was hydrolysed according to the general procedure described in Example 1 above, except that the volume of solvent was 3 mL and the reaction time was 16 hours. The crude acid obtained thereby was dissolved in DCM (2 mL) and the amide coupling (with (2-Aminomethyl-4-chlorobenzyl)-carbamic acid tert-butyl ester) was performed as described in Example 1 above, except that the reaction mixture was stirred for two nights. The crude product was purified by chromatography (SiO₂, 5% methanol in DCM) and preparative HPLC to give the title compound (41%).

¹H NMR (500 MHz, CDCl₃) δ 1.50 (s, 9H), 2.01 (s, 3H), 2.48-2.63 (m, 2H), 3.02 (s, 2H), 3.72-3.79 (m, 2H), 4.07 (d, 2H), 4.19-4.29 (m, 2H), 5.10 (br s, 1H), 6.20 (br s, 1H), 6.79 (br s, 1H), 7.23-7.26 (m, 2H), 7.35 (t, 1H), 7.60 (t, 1H), 7.71 (t, 1H), 7.85 (d, 1H), 7.93 (d, 1H), 8.26 (d, 1H), 8.81 (d, 1H)

### Example 4

### N-(2-Aminomethyl-5-chlorobenzyl)-2-[4-methyl-1-(naphthalene-1-sulfonylamino)-2-oxo-1,2,5,6-tetrahydropyridin-3-yl]acetamide, hydrochloride salt

[4-Chloro-2-({2-[4-methyl-1-(naphthalene-1-sulfonylamino)-2-oxo-1,2,5,6-tetrahydro-pyridin-3-yl]acetylamino}methyl)benzyl]carbamic acid tert-butyl ester (0.019 mmol; see Example 3 above) was dissolved in ethyl acetate saturated with HCl (1 mL) and stirred at room temperature for 30 minutes. The solvent and excess reagents were evaporated under is reduced pressure to give the title compound (76%).

¹H NMR (500 MHz, CDCl₃) δ 1.86 (s, 3H), 2.40 (t, 2H), 3.03 (s, 2H), 3.39 (t, 2H), 4.18 (s, 2H), 4.26 (s, 2H), 7.30 (d, 1H), 7.35-7.41 (m, 2H), 7.55 (t, 1H), 7.62 (t, 1H), 7.69 (t, 1H), 7.99 (d, 1H), 8.16 (d, 1H), 8.25 (d, 1H), 8.79 (d, 1H).

MS m/z 527 (M+H)⁺

### Example 5

### N-{5-Chloro-2-[(cyclopentylamino)methyl]benzyl}-2-{4-methyl-1-[(1-naphthalsulfonyl)amino]-2-oxo-1,2,5,6-tetrahydrolpyridin-3-yl}acetamide

TEA (0.5 mL of a solution of 0.14 mL in 5 mL of DCM) and cyclopentanone (0.5 mL of a solution of 0.5 mL in 5 mL of DCM) were added to a solution of N-(2-aminomethyl-5-chlorobenzyl)-2-[4-methyl-1-(naphthalene-1-sulfonylamino)-2-oxo-1,2,5,6-tetrahydropyridin-3-yl]-acetamide, hydrochloride salt (0.091 mmol; see Example 4 above) in DCM (4 mL). Sodium triacetoxyborohydride (0.302 mmol) was added, and the resulting suspension was stirred at room temperature for 3.5 hours. The reaction mixture was added to a SCX-2 ion-exchange column that had been pre-washed with THF. After washing with 12 mL methanol, NH₃ in methanol was used to wash out the product. The solvent was removed under reduced pressure to give the title compound.

¹H NMR (500 MHz, CD₃OD) δ 8.85 (d, 1H), 8.24 (d, 1H), 8.10 (d, 1H), 7.95 (d, 1H), 7.67 (t, 1H), 7.60 (t, 1H), 7.48 (t, 1H), 7.34 (d, 1H), 7.26 (dd, 1H), 7.13 (d, 1H), 4.24 (s, 2H), 3.80 (s, 2H), 3.53 (t, 2H), 3.19 (p, 1H), 3.03 (s, 2H), 2.47 (t, 2H), 1.85-2.00 (m, 5H), 1.68-1.79 (m, 2H), 1.53-1.64 (m, 2H), 1.42-1.52 (m, 2H)

MS m/z 597 (M+H)⁺

### Example 6

### N-{2-[(Cyclopentylamino)methyl]benzyl}-2-{4-methyl-1-[(1-naphthylsulfonyl)amino]-2-oxo-1,2,5,6-tetrahydropyridin-3-yl}acetamide acetate

The title compound was prepared from the product of Preparation 2(xiii) by using procedures analogous to those described in Example 5 above.

¹H NMR (500 MHz, CD₃OD) δ 8.82 (d, 1H), 8.26 (d, 1H), 8.19 (d, 1H), 8.01 (d, 1H), 7.62-7.74 (m, 2H), 7.57 (t, 1H), 7.29-7.48 (m, 4H), 4.27 (s, 4H), 3.59-3.67 (m, 1H), 3.43 (t, 2H), 3.04 (s, 2H), 2.42 (t, 2H), 2.11-2.21 (m, 2H), 1.93 (s, 3H), 1.66-1.88 (m, 8H)

MS m/z 562 (M+H)⁺

### Example 7

### 2-(1-{[(5-Chloro-1,3-dimethyl-1H-pyrazol-4-yl)methyl]amino}-4-methyl-2-oxo-1,2,5,6-tetrahydropyridin-3-yl)-N-(5-chloro-2-{[(2,2,2-trifluoroethyl)-amino]methyl}benzyl)acetamide

Crude N-[2-(aminomethyl)-5-chlorobenzyl]-2-(1-{[(5-chloro-1,3-dimethyl-1H-pyrazol-4-yl)methyl]amino}-4-methyl-2-oxo-1,2,5,6-tetrahydropyridin-3-yl)acetamide (10 mg, 0.02 mmol, see Example 2(xxix) above) was dissolved in dry toluene (1.0 mL). 1-Ethoxy-2,2,2-trifluoroethanol (4.2 mg, 0.03 mmol, 1.5 eq.) was added and the mixture was heated for 2 hours at 100° C. before being concentrated. The resulting imine was dissolved in a mixture of methanol (1 mL) and acetic acid (0.25 mL), to which NaBH₃CN (3.7 mg, 2.85 mmol, 3.0 eq.) was then added. The reaction mixture was stirred at room temperature overnight before being partitioned between sodium hydrogencarbonate (sat.) and DCM. The mixture was extracted with DCM (3×) and the organic phase was dried through a phase separator and the solvent was evaporated under reduced pressure. Purification by Prep-HPLC gave the title compound.

¹H NMR (500 MHz, CDCl₃) δ 2.05 (s, 3H), 2.26 (s, 3H), 2.40 (t, 2H), 3.24 (q, 2H), 3.32 (s, 2H), 3.42 (t, 2H), 3.76 (s, 3H), 3.80 (s, 2H), 3.91 (s, 2H), 4.45 (d, 2H), 7.19-7.22 (m, 2H), 7.23-7.25 (m, 1H), 7.45-7.51 (m, 1H)

MS m/z 561 (M+H)⁺

### Example 8

The compounds (i) and (ii) listed below were prepared from the compounds of Example 1(xxx) and Example 2(xxiii), respectively, by prolonged exposure to ethyl acetate saturated with HCl (according to Example 2, Method B above). The title compounds were then isolated by Prep-HPLC.

### (i) N-[5-chloro-2-(1H-tetrazol-1-yl)benzyl]-2-(4-methyl-2-oxo-1-{[(2-oxo-1,2-dihydropyridin-3-yl)methyl]amino}-1,2,5,6-tetrahydropyridin-3-yl)-acetamide

¹H NMR (500 MHz, CD₃OD) δ 9.55(s, 1H), 7.60 (d, 1H), 7.51-7.55 (m, 2H), 7.47 (d, 1H), 7.31 (dd, 1H), 6.25 (t, 1H), 4.16 (s, 2H), 3.86 (s, 2H), 3.53 (t, 2H), 3.21 (s, 2H), 2.50 (t, 2H), 1.91 (s, 3H)

MS m/z 485 (M+H)⁺

### (ii) N-[(6-Amino-2,4-dimethylpyridin-3-yl)methyl]-2-(4-methyl-2-oxo-1-{[(2-oxo-1,2-dihydropyridin-3-yl)methyl]amino}-1,2,5,6-tetrahydropyridin-3-yl)acetamide

¹H NMR (500 MHz, CD₃OD) δ 1.93 (s, 3H), 2.26 (s, 3H), 2.39 (s, 3H), 2.48 (t, 2H), 3.23 (s, 2H), 3.52 (t, 2H), 3.85 (s, 2H), 4.27 (s, 2H), 6.32 (t, 1H), 6.34 (s, 1H), 7.35 (dd, 1H), 7.54 (dd, 1H)

MS m/z 425 (M+H)⁺

### Example 9

Compounds of the Examples were tested in Test B above and were found to exhibit IC₅₀ values of less than 50 μM. Indeed, the compounds of Examples 2(i) and 2(iii) were found to exhibit IC₅₀ values of 0.24 μM and 25.6 nM, respectively.

Abbreviations

aq.=aqueous
AUC=area under the curve
Boc=tert-butyloxycarbonyl
BSA=bovine serum albumin
mCPBA=meta-chloroperbenzoic acid
d=(in relation to NMR) doublet
DCC=dicyclohexyl carbodiimide
DCM=dichloromethane
DIPEA=diisopropylethylamine
DMAP=4-(N,N-dimethyl amino) pyridine
DMF=dimethylformamide
DMSO=dimethylsulfoxide
DVT=deep vein thrombosis
EDC=1-(3-dimethylaminopropyl)-3-ethylcarbodiimide hydrochloride
Et=ethyl
ether=diethyl ether
EtOAc=ethyl acetate
EtOH=ethanol
Et₂O=diethyl ether
h=hour(s)
HATU=O-(azabenzotriazol-1-yl)-N,N,N′,N′-tetramethyluronium hexafluorophosphate
HBTU=[N,N,N′,N′-tetramethyl-O-(benzotriazol-1-yl)uronium hexafluorophosphate]
HCl=hydrochloric acid, hydrogen chloride gas or hydrochloride salt (depending on context)
HOAT=1-hydroxy-7-azabenzotriazole
HOBT=1-hydroxybenzotriazole
HPLC=high performance liquid chromatography
HRMS=high resolution mass spectroscopy
LC=liquid chromatography
Me=methyl
MeOH=methanol
min=minute(s)
MS=mass spectroscopy
NADH=nicotinamide adenine dinucleotide, reduced form
NADPH=nicotinamide adenine dinucleotide phosphate, reduced form
NIH=National Institute of Health (US)
NIHU=National Institute of Health units
OAc=acetate
PCC=pyridinium chlorochromate
Ph=phenyl
Pr=propyl
PyBOP=(benzotriazol-1-yloxy)tripyrrolidinophosphonium hexafluorophosphate
rt/RT=room temperature
SOPs=standard operating procedures
TBTU=[N,N,N′,N′-tetramethyl-O-(benzotriazol-1-yl)uronium tetrafluoroborate]
TEA=triethylamine
Teoc=2-(trimethylsilyl)ethoxycarbonyl
TFA=trifluoroacetic acid
THF=tetrahydrofuran

Prefixes n, s, i and t have their usual meanings: normal, secondary, iso and tertiary. The prefix c means cyclo.

1. A compound of formula I 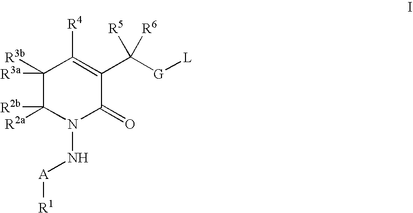
wherein
A represents C(O), S(O)₂, C(O)O (in which latter group the O moiety is attached to R¹), C(O)NH, S(O)₂NH (in which latter two groups the NH moiety is attached to R¹) or C₁₋₆ alkylene;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{7a}, S(O)R^{7b}, S(O)₂N(R^{7c})(R^{7d}), N(R^{7e})S(O)₂R^{7f}, N(R^{7g})(R^{7h}), B¹—C(O)—B²—R⁷ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{7a}, S(O)R^{7b}, S(O)₂N(R^{7c})(R^{7d}), N(R^{7e})S(O)₂R^{7f}, N(R^{7g})(R^{7h}), B³—C(O)—B⁴—R⁷ⁱ, aryl and Het²,
(c) aryl, or
(d) Het³;
R^{7a} to R⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{7b} does not represent H when n is 1 or 2;
R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene;
R⁴ represents
(a) H,
(b) halo,
(c) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl),
(d) together with R⁵, R⁴ represents C₂₋₃ n-alkylene, T¹-(C₁₋₂ n-alkylene) or (C₁₋₂ n-alkylene)-T¹, which latter three groups are optionally substituted by halo, or
(e) together with R⁵ and R⁶, R⁴ represents T²-[C(H)═], wherein T² is bonded to the C-atom to which the group R⁴ is attached;
R⁵ and R⁶ independently represent H, F or methyl (which latter group is optionally substituted by one or more F atoms), or
(a) together with R⁴, R⁵ represents C₂₋₃ n-alkylene, T¹-(C₁₋₂ n-alkylene) or (C₁₋₂ n-alkylene)-T¹, which latter three groups are optionally substituted by halo, or
(b) together with R⁴, R⁵ and R⁶ represents T²-[C(H)═], wherein T² is bonded to the C-atom to which the group R⁴ is attached;
T¹ and T² independently represent O, S, N(H) or N(C₁₋₄ alkyl);
G represents
(a) —C(O)N(R^{8a})—[CH(C(O)R⁹)]₀₋₁—C₀₋₃ alkylene-(Q¹)ₐ-,
(b) —C(O)N(R^{8b})—C₂₋₃ alkenylene-(Q¹)ₐ-, 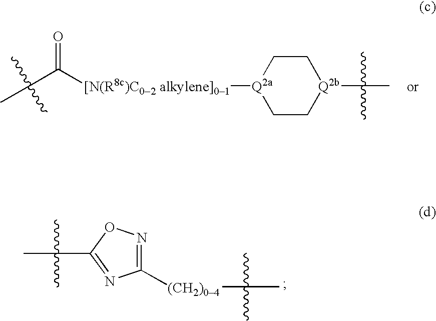
R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl;
Q¹ represents O, NR^{10a}, [N(H)]₀₋₁C(O)—C₀₋₂ alkylene, C(O)NHNHC(O), or —N═C(R^{10b})—;
a represents 0 or 1;
Q^{2a} represents 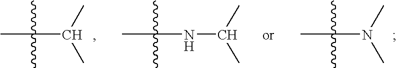
Q^{2b} represents 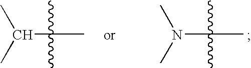
L represents
(a) C₀₋₆ alkylene-R^{a},
(b) C₀₋₂ alkylene-CH═CH—C₀₋₂ alkylene-R^{a},
(c) C₀₋₂ alkylene-C≡C—C₀₋₂ alkylene-R^{a}, 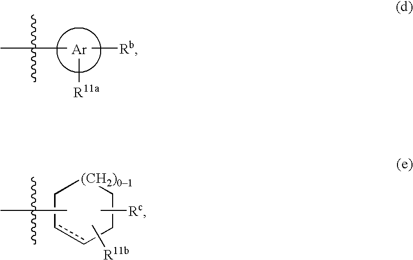
wherein the dashed line represents an optional double bond, or 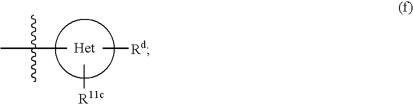
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}) and S(O)₂R^{12d};
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}), S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent 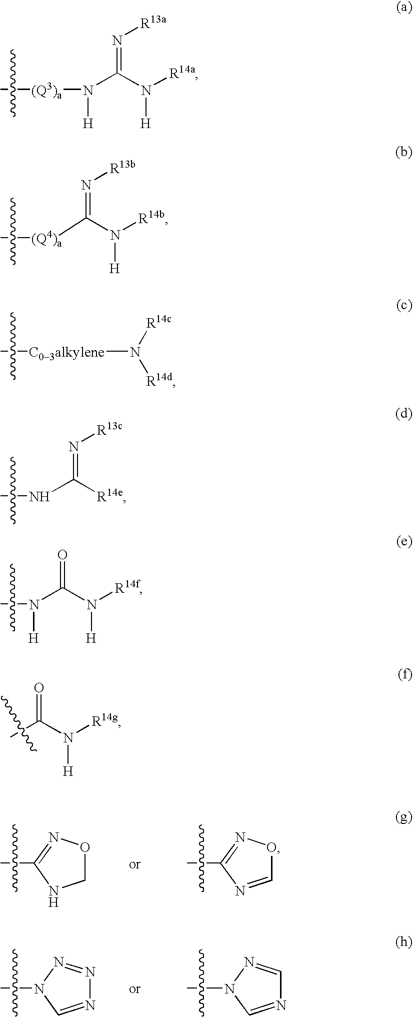
or R^{b} to R^{d} may also represent H;
Q³ represents O, N(R^{10c}), S(O)₂, S(O)₂NH, C(O) or —CH═N—;
Q⁴ represents O, S or CH₂;
a represents 0 or 1;
R^{13a} to R^{13c} independently represent
(a) H,
(b) CN,
(c) NH₂,
(d) OR¹⁵ or
(e) C(O)OR¹⁶;
R¹⁵ represents
(a) H,
(b) C₁₋₁₀ alkyl, C₃₋₁₀ alkenyl, C₃₋₁₀ alkynyl,
(c) C₃₋₁₀ cycloalkyl, C₄₋₁₀ cycloalkenyl, which latter two groups are optionally substituted by one or more substituents selected from halo and C₁₋₆ alkyl, or
(d) C₁₋₃ alkyl, which latter group is optionally interrupted by oxygen and is substituted by aryl or —O-aryl;
R¹⁶ represents
(a) C₁₋₁₀ alkyl, C₃₋₁₀ alkenyl, C₃₋₁₀ alkynyl, which latter three groups are optionally interrupted by one or more oxygen atoms, or
(b) C₃₋₁₀ cycloalkyl, C₄₋₁₀ cycloalkenyl, which latter two groups are optionally substituted by one or more substituents selected from halo and C₁₋₆ alkyl, or
(c) C₁₋₃ alkyl, which latter group is optionally interrupted by oxygen and is substituted by aryl or —O-aryl;
R^{8a} to R^{8c}, R^{10a} to R^{10c} and R^{14a} to R^{14g} independently represent
(a) H or
(b) C₁₋₄ alkyl (which latter group is optionally substituted by one or more substituents selected from halo and OH),
or R^{14a} and R^{14b} independently represent C(O)O—C₁₋₆ alkyl (the alkyl part of which latter group is optionally substituted by aryl and/or one or more halo atoms),
or R^{14c} represents
(a) C₁₋₄ alkyl substituted by C₃₋₇ cycloalkyl or aryl,
(b) C₃₋₇ cycloalkyl,
(c) C(O)O—C₁₋₆ alkyl (the alkyl part of which latter group is optionally substituted by aryl and/or one or more halo atoms),
(d) C(O)C₁₋₆ alkyl,
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
or R^{14c} and R^{14d} together represent C₃₋₆ n-alkylene optionally interrupted by O, S, N(H) or N(C₁₋₄ alkyl) and/or substituted by one or more C₁₋₄ alkyl groups;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c});
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
B¹ to B⁸ independently represent a direct bond, O, S or NH;
n, p and q independently represent 0, 1 or 2;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl);
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a pharmaceutically-acceptable derivative thereof.

2. A compound as claimed in claim 1 which is a compound of formula Ia, Ib or Ic 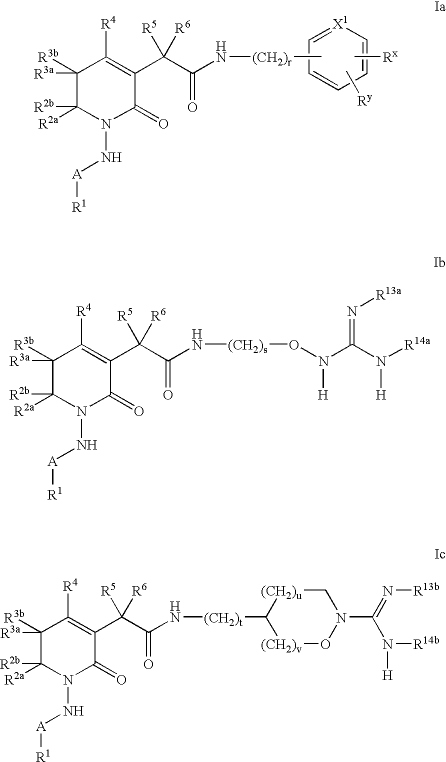
wherein X¹ represents CH or N;
when X¹ represents CH
(a) R^{x} represents R^{b} as defined in claim 1, and
(b) R^{y} represents R^{11a} as defined claim 1;
when X¹ represents N
(a) R^{x} represents R^{d} as defined in claim 1, and
(b) R^{y} represents R^{11c} as defined in claim 1;
r represents 1 to 3;
s represents 2 to 4; and
t represents 1 to 3;
u and v independently represent 0 to 2, the sum of u and v being 1 or 2;
R¹, R^{2a}, R^{2b}, R^{3a}, R^{3b}, R⁴, R⁵, R⁶, R^{11a}, R^{11c}, R^{13a}, R^{14a}, R^{14b}, R^{b}, R^{d} and A are as defined in claim 1.

3. A compound as claimed in claim 2 which is a compound of formula Ia 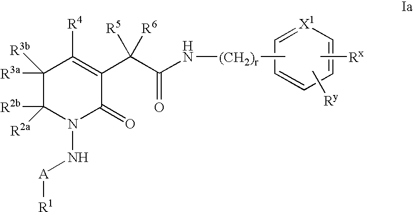
wherein
A represents CH₂, (CH₂)₂ or CF₂CH₂ (in which latter group the CF₂ unit is attached to R¹);
R¹ represents
(a) phenyl optionally substituted by one or two substituents selected from halo, methyl, CF₃ and methoxy,
(b) pyrazolyl optionally substituted by one to three substituents selected from Cl and methyl,
(c) thienyl optionally substituted by Cl or pyridinyl,
(d) pyridinyl optionally substituted by OH or methoxy,
(e) pyridonyl or
(f) benzodioxolyl optionally substituted by halo;
R^{2a}, R^{2b}, R^{3a}, R^{3b} all represent H;
R⁴ represents methyl;
r represents 1;
R⁵ and R⁶ both represent H;
the group 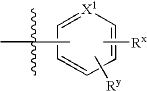
represents 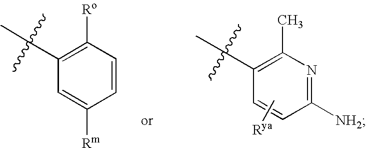
R^{o} represents H, F, Cl, OH, methyl, tetrazol-1-yl, OCH₂C(O)N(H)R^{12b} or CH₂N(H)R^{14c};
R^{12b} represents H or C₁₋₃ alkyl optionally substituted by N(CH₃)₂;
R^{14c} represents C(O)O-tert-butyl, H, ethyl, CH₂CF₃ or cyclopentyl;
R^{m} represents H, methyl, CF₃, methoxy, F or Cl; and
R^{ya} represents H or methyl.

4. A pharmaceutical formulation including a compound as defined in any one of claims 1 to 3, or a pharmaceutically acceptable derivative thereof, in admixture with a pharmaceutically acceptable adjuvant, diluent or carrier.

5. A compound as defined in any one of claims 1 to 3, or a pharmaceutically acceptable derivative thereof, for use as a pharmaceutical.

6. (canceled)

7. A method of treatment of a condition where inhibition of thrombin is beneficial, which method comprises administration of a therapeutically effective amount of a compound as defined in any one of claims 1 to 3, or a pharmaceutically acceptable derivative thereof, to a person suffering from, or susceptible to, such a condition.

8. A process for the preparation of a compound of formula I as defined in claim 1, which comprises:
(a) for compounds of formula I in which the group G represents
(i) C(O)N(R^{8a})—[CH(C(O)R⁹)]₀₋₁—C₀₋₃ alkylene-(Q¹)ₐ-,
(ii) C(O)N(R^{8b})—C₂₋₃ alkenylene-(Q¹)ₐ-,
(iii) C(O)N(R^{8b})—C₂₋₃ alkynylene-(Q¹)ₐ-, 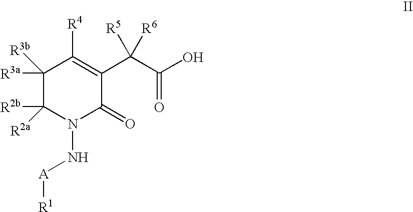
wherein Q^{2a} represents N or NHCH,
coupling of a compound of formula II, 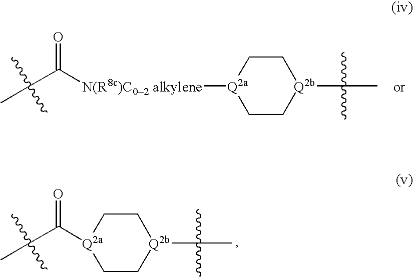
wherein R¹, R^{2a}, R^{2b}, R^{3a}, R^{3b}, R⁴, R⁵, R⁶ and A are as defined in claim 1, with a compound of formula III,
H-G^{a}-L III
wherein L is as defined in claim 1 and G^{a} represents
(i) —N(R^{8a})—[CH(C(O)R⁹)]₀₋₁—C₀₋₃ alkylene-(Q¹)ₐ-,
(ii) —N(R^{8b})—C₂₋₃ alkenylene-(Q¹)ₐ-,
(iii) —N(R^{8b})—C₂₋₃ alkynylene-(Q¹)ₐ-, 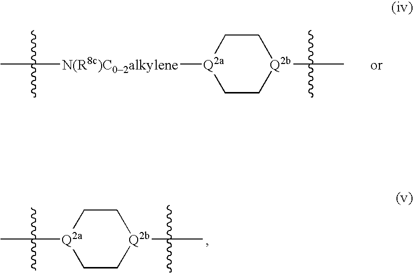
wherein Q^{2a} represents N or NHCH and R^{8a}, R^{8b}, R^{8c}, R⁹, Q¹, Q^{2b} and a are as defined in claim 1;
(b) for compounds of formula I in which G represents 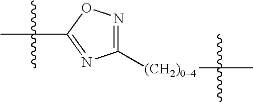
and L represents L^{a}, which latter group represents L as defined in claim 1, except that it does not represent C₀ alkylene-R^{a}, cyclisation of a compound of formula IV, 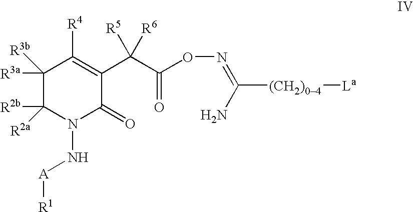
wherein L^{a} is as defined above and R¹, R^{2a}, R^{2b}, R^{3a}, R^{3b}, R⁴, R⁵ and A are as defined in claim 1;
(c) for compounds of formula I in which R^{a}, R^{b}, R^{c} or R^{d} represents —C(═NH)NH₂, —C(═NHNH₂)NH₂ or —C(═NOH)NH₂, reaction of a compound of formula V, 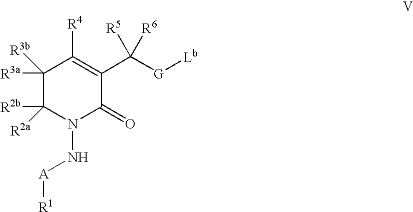
wherein L^{b} represents L as defined in claim 1, except that R^{a}, R^{b}, R^{c} or R^{d} (as appropriate) is replaced by a cyano or —C(═NH)O—C₁₋₄ alkyl group, and R¹, R^{2a}, R^{2b}, R^{3a}, R^{3b}, R⁴, R⁵, R⁶, G and A are as defined in claim 1, with a suitable source of ammonia, hydrazine or hydroxylamine;
(d) for compounds of formula I in which R^{13a}, R^{13b} or R^{13c} represents H, deprotection of a corresponding compound of formula I in which R^{13a}, R^{13b} or R^{13c} (as appropriate) represents C(O)O—CH₂aryl;
(e) for compounds of formula I in which R^{14c} represents H, deprotection of a corresponding compound of formula I in which R^{14c} represents C(O)O—C₁₋₆ alkyl;
(f) reaction of a compound of formula VI, 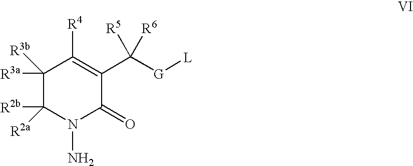
wherein R^{2a}, R^{2b}, R^{3a}, R^{3b}, R⁴, R⁵, R⁶, G and L are as defined in claim 1, with a compound of formula VII,
R¹-A-Lg² VII
wherein Lg² represents a leaving group and R¹ and A are as defined in claim 1;
(g) for compounds of formula I in which A represents C(O)NH, reaction of a compound of formula VI, as defined above, with a compound of formula VIII,
R¹—N═C═O VIII
wherein R¹ is as defined in claim 1;
(h) for compounds of formula I in which A represents C₁₋₆ alkylene, reaction of a compound of formula VI, as defined above, with a compound of formula IX,
R¹—C₀₋₅ alkylene-CHO IX
wherein R¹ is as defined in claim 1, followed by reduction in the presence of a reducing agent; or
(i) for compounds of formula I in which R^{a}, R^{b}, R^{c} or R^{d} represents —C(═NCN)NH₂, reaction of a corresponding compound of formula I in which R^{a}, R^{b}, R^{c} or R^{d}, respectively, represents —C(═NH)NH₂ with cyanogen bromide.

9. A compound of formula II 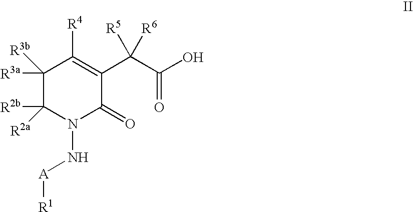
wherein
R¹ represents
(a) C₁₋₁₀ alkyl C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl) OR^{7a}, S(O)ₙR^{7b}, S(O)₂N(R^{7c})(R^{7d}), N(R^{7e})S(O)₂R^{7f}, N(R^{7g})(R^{7h}), B¹—C(O)—B²—R⁷ⁱ, aryl and Het¹),
(b) C₃₋₁₀ cycloalkyl or C₄ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{7a}, S(O)ₙR^{7b}, S(O)₂N(R^{7c})(R^{7d}), N(R^{7e})S(O)R^{7f}, N(R^{7g})(R^{7h}), B³—C(O)—B⁴—R⁷ⁱ, aryl and Het²,
(c) aryl, or
(d) Het³;
R^{7a} to R⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶, provided that R^{7b} does not represent H when n is 1 or 2;
Het¹ to Het⁶ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(h) S(O)₂N(R^{19c})(R^{19d}),
(i) N(R^{19e})S(O)₂R^{19f},
(j) N(R^{19g})(R^{19H}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C_{—}₆ alkyl;
B¹ to B⁴, B⁷, and B⁸ independently represent a direct bond, O, S or NH;
n and q independently represent 0, 1 or 2;
R^{20a}, R^{20b} and R^{20c} independently represent C₁₋₆ alkyl or phenyl (which latter group is optionally substituted by halo or C₁₋₄ alkyl);
R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene,
R⁴ represents
(a) H,
(b) halo,
(c) C₁₋₆ alkyl, C₂₋₆ alkenyl, C₂₋₆ alkynyl, C₂₋₆ alkoxy (which latter four groups are optionally substituted by one or more substituents selected from halo, OH, CN, C₁₋₄ alkoxy C(O)OH, C(O)O—C₁₋₄ alkyl and OC(O)—C₁₋₄ alkyl),
(d) together with R⁵, R⁴ represents C₂₋₃ n-alkylene, T¹-(C₁₋₂ n-alkylene) or (C₁₋₂ n-alkylene)-T¹ which latter three groups are optionally substituted by halo, or
(e) together with R⁵ and R⁶, R⁴ represents T²-[C(H)═], wherein T² is bonded to the C-atom to which the group R⁴ is attached,
R⁵ and R⁶ independently represent H, F or methyl (which latter group is optionally substituted by one or more F atoms), or
(a) together with R⁴, R⁵ represents C_{—-3} n-alkylene, T¹-(C₁₋₂ n-alkylene) or (C₁₋₂ n-alkylene)-T¹ which latter three groups are optionally substituted by halo, or
(b) together with R⁴, R⁵ and R⁶ represents T²-[C(H)═], wherein T² is bonded to the C-atom to which the group R⁴ is attached;
T¹ and T² independently represent O, S, N(H) or N(C₁₋₄ alkyl), and
A represents C(O), S(O)₂, C(O)O (in which latter group the 0 moiety is attached to R¹), C(O)NH, S(O)₂NH (in which latter two groups the NH moiety is attached to R¹) or C₁₋₆ alkylene; unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

10. A compound of formula I, a defined in claim 8, 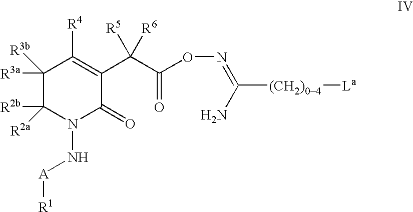
wherein
R¹ represents
(a) C₁₋₁₀ alkyl, C₂₋₁₀ alkenyl, C₂₋₁₀ alkynyl (which latter three groups are optionally substituted by one or more substituents selected from halo, CN, Cam cycloalkyl (optionally substituted by one or more substituents selected from halo, OH, ═O, C₁₋₆ alkyl, C₁₋₆ alkoxy and aryl), OR^{7a}, S(O)₂R^{7b}, S(O)₂N(R^{7c})(R^{7d}), N(R^{7e})S(O)₂R^{7f}, N(R^{7g})(R^{7h}), B¹—C(O)—B²—R⁷ⁱ aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl) OR^{7a}, S(O)ₙR^{7b}, S(O)₂N(R^{7c})(R^{7d}), N(R^{7e})S(O)₂R^{7f}, N(R^{7g})(R^{7h}), B³—C(O)—B⁴—R⁷ⁱ, aryl and Het²,
(c) aryl, or
(d) Het³;
R^{7a} to R⁷ⁱ independently represent, at each occurrence,
(a) H,
(b) C₁₋₁₀ alkyl, C₂₋₁₀ alkenyl C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{7b} does not represent H when n is 1 or 2;
Het¹ to Het⁶ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C_{—}₆ alkyl, B¹ to B⁴, B⁷, and B⁸ independently represent a direct bond, O, S or NH;
n and q independently represent 0, 1 or 2;
R^{20a}, R^{20b} and R^{20c} independently represent C₁₋₆ alkyl or phenyl (which latter group is optionally substituted by halo or C₁₋₄ alkyl);
R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene
R⁴ represents
(a) H,
(b) halo,
(c) C₁₋₆ alkyl C₂₋₆ alkenyl C₂₋₆ alkynyl C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl),
(d) together with R⁵, R⁴ represents C₂₋₃ n-alkylene, T¹-(C₁₋₂ n-alkylene) or (C₁₋₂ n-alkylene)-T¹ which latter three groups are optionally substituted by halo, or
(e) together with R⁵ and R⁶, R⁴ represents T²-[C(H)═], wherein T² is bonded to the C-atom to which the group R⁴ is attached,
R⁵ and R⁶ independently represent H, F or methyl (which latter group is optionally substituted by one or more F atoms), or
(a) together with R⁴, R⁵ represents C₂₋₃ n-alkylene, T¹-(C₁₋₂ n-alkylene) or (C₁₋₂ n-alkylene)-T¹ which latter three groups are optionally substituted by halo, or
(b) together with R⁴, R⁵ and R⁶ represents T²-[C(H)═], wherein T² is bonded to the C-atom to which the group R⁴ is attached;
T¹ and T² independently represent O, S, N(H) or N(C₁₋₄ alkyl), and
A represents C(O), S(O)₂, C(O)O (in which latter group the O moiety is attached to R¹), C(O)NH, S(O)₂NH (in which latter two groups the NH moiety is attached to R¹) or C₁₋₆ alkylene,
L^{a} represents
(a) C₁₋₆ alkylene-R^{a},
(b) C₀₋₂ alkylene-CH═CH—C₀₋₂ alkylene-R^{a},
(c) C₀₋₂ alkylene-C≡C—C₀₋₂ alkylene-R^{a}, 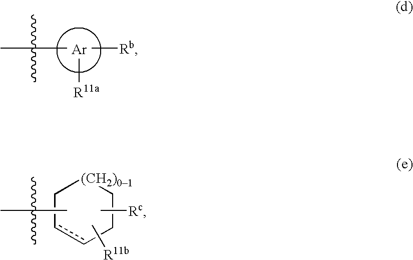
wherein the dashed line represents an optional double bond, or 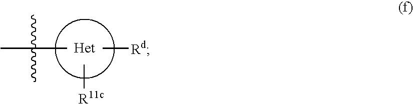
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl C₁₋₆ alkoxy (which latter two groups are optionally substituted by one or more substituents selected from halo, OH, C₁₋₄ alkoxy, C(O)OR^{12a} and C(O)N(R^{12b})R^{12c}) and S(O)₀₋₂R^{12d},
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}), S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent 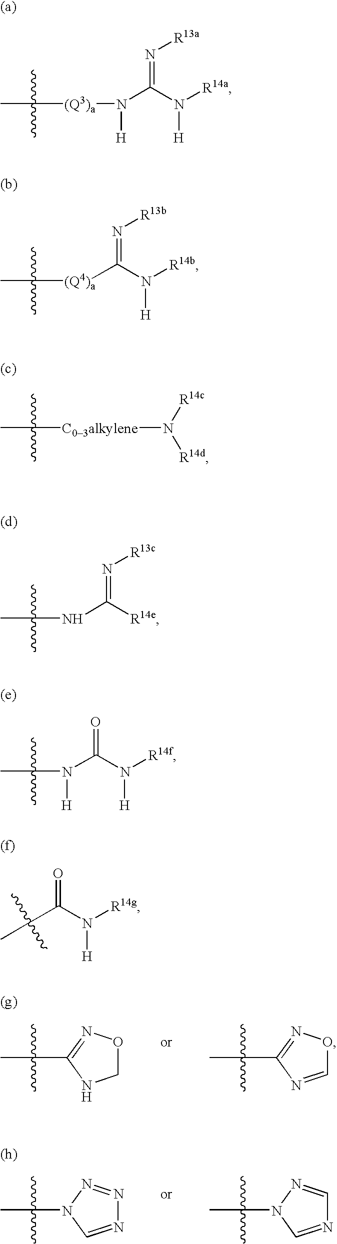
or R^{b} to R^{d} may also represent H;
Q³ represents O, N(R^{10c}) S(O)₂, S(O)₂NH, C(O) or —CH═N—;
Q⁴ represents O, S or CH₂;
a represents 0 or 1;
R^{13a} to R^{13c} independently represent
(a) H,
(b) CN,
(c) NH₂,
(d) OR¹⁴ or
(e) C(O)OR¹⁶;
R¹⁵ represents
(a) H,
(b) C₁₋₁₀ alkyl, C₃₋₁₀ alkenyl C₃₋₁₀ alkynyl
(c) C₃₋₁₀ cycloalkyl, C₄₋₁₀ cycloalkenyl, which latter two groups are optionally substituted by one or more substituents selected from halo and C₁₋₆ alkyl, or
(d) C₁₋₃ alkyl, which latter group is optionally interrupted by oxygen and is substituted by aryl or —O-aryl,
R¹⁶ represents
(a) C₁₋₁₀ alkyl, C₃₋₁₀ alkenyl, C₃₋₁₀ alkynyl, which latter three groups are optionally interrupted by one or more oxygen atoms, or
(b) C₃₋₁₀ cycloalkyl, C₄₋₁₀ cycloalkenyl, which latter two groups are optionally substituted by one or more substituents selected from halo and C₁₋₆ alkyl, or
(c) C₁₋₃ alkyl, which latter group is optionally interrupted by oxygen and is substituted by aryl or —O-aryl,
R^{10c} and R^{14a} to R¹⁴, independently represent
(a) H or
(b) C₁₋₄ alkyl (which latter group is optionally substituted by one or more substituents selected from halo and OH),
or R^{14a} and R^{14b} independently represent C(O)O—C₁₋₆ alkyl (the alkyl part of which latter group is optionally substituted by aryl and/or one or more halo atoms),
or R^{14c} represents
(a) C₁₋₄ alkyl substituted by C₃₋₇ cycloalkyl or aryl,
(b) C₃₋₇ cycloalkyl,
(c) C(O)O—C₁₋₆ alkyl (the alkyl part of which latter group is optionally substituted by aryl and/or one or more halo atoms),
(d) C(O)C₁₋₆ alkyl,
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
or R^{14c} and R^{14d} together represent C₃₋₆ n-alkylene optionally interrupted by O, S, N(H) or N(C₁₋₄ alkyl) and/or substituted by one or more C₁₋₄ alkyl groups, unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

11. A compound of formula V, as defined in claim 8, 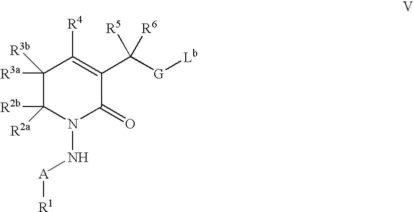
wherein
R¹ represents
(a) C₁₋₁₀ alkyl C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{7a}, S(O)ₙR^{7b}, S(O)₂N(R^{7c})(R^{7d}), N(R^{7e})S(O)₂R^{7f}, N(R^{7g})(R^{7b}), B¹—C(O)—B²—R⁷ⁱ aryl and Het¹),
(b) Cow cycloalkyl or C₄ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{7a}, S(O)ₙR^{7b}, S(O)₂N(R^{7c})(R^{7d}), N(R^{7e})S(O)₂R^{7f}, N(R^{7g})(R^{7h}), B³—C(O)—B⁴—R⁷ⁱ, aryl and Het²,
(c) aryl, or
(d) Het³;
R^{7a} to R⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{7b} does not represent H when n is 1 or 2;
Het¹ to Het⁶ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C_{—}₆ alkyl;
B¹ to B⁴, B⁶ and B⁷ independently represent a direct bond, O, S or NH;
n and q independently represent 0, 1 or 2;
R^{20a}, R^{20b} and R^{20c} independently represent C₁₋₆ alkyl or phenyl (which latter group is optionally substituted by halo or C₁₋₄ alkyl);
R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene,
R⁴ represents
(a) H,
(b) halo,
(c) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl),
(d) together with R⁵, R⁴ represents C_{—-3} n-alkylene, T¹-(C₁₋₂ n-alkylene) or (C₁₋₂ n-alkylene)-T¹ which latter three groups are optionally substituted by halo, or
(e) together with R⁵ and R⁶, R⁴ represents T²-[C(H)═], wherein T is bonded to the C-atom to which the group R⁴ is attached,
R⁵ and R⁶ independently represent H, F or methyl (which latter group is optionally substituted by one or more F atoms), or
(a) together with R⁴, R⁵ represents C_{—-3} n-alkylene, T¹-(C₁₋₂ n-alkylene) or (C₁₋₂ n-alkylene)-T¹, which latter three groups are optionally substituted by halo, or
(b) together with R⁴, R⁵ and R⁶ represents T²-[C(H)═], wherein T is bonded to the C-atom to which the group R⁴ is attached;
T¹ and T² independently represent O, S, N(H) or N(C₁₋₄ alkyl); and
A represents C(O), S(O)₂, C(O)O (in which latter group the O moiety is attached to R¹), C(O)NH, S(O)NH (in which latter two groups the NH moiety is attached to R¹) or C₁₋₆ alkylene;
G represents
(a) —C(O)N(R^{8a})—[CH(C(O)R⁹)]₀₋₁—C₀₋₃ alkylene-(Q¹)ₐ-,
(b) —C(O)N(R^{8b})—C₂₋₃ alkenylene-(Q¹)ₐ-, 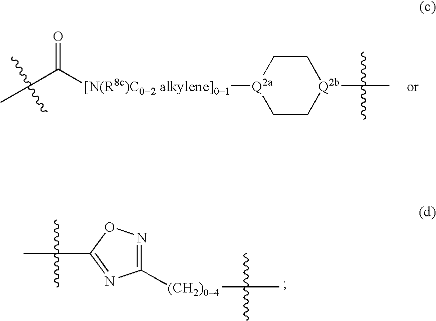
R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl;
Q¹ represents O, NR^{10a}, [N(H)]₀₋₁C(O)—C₀₋₂ alkylene, C(O)NHNHC(O), or —N═C(R^{10b})—;
a represents 0 or 1;
Q^{2a} represents 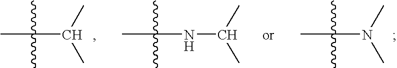
Q^{2b} represents 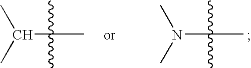
R^{8a} to R^{8c}, R^{10a} to R^{10b} independently represent
(a) H or
(b) C₁₋₄ alkyl (which latter group is optionally substituted by one or more substituents selected from halo and OH),
L^{b} represents
(a) C₀₋₆ alkylene-R^{a},
(b) C₀₋₂ alkylene-CH═CH—C₀₋₂ alkylene-R^{a},
(c) C₀₋₂ alkylene-C≡C—C₀₋₂ alkylene-R^{a}, 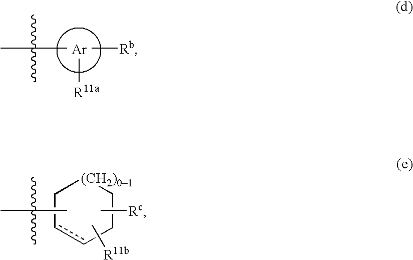
wherein the dashed line represents an optional double bond, or 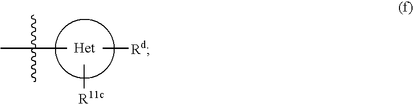
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl C₁₋₆ alkoxy (which latter two groups are optionally substituted by one or more substituents selected from halo, OH, C₁₋₄ alkoxy, C(O)OR^{12a} and C(O)N(R^{12b})R^{12c}) and S(O)₀₋₂R^{12d};
R^{11b} and R^{11c} independently represent H or one or more substituents selected from halo, OH, CN, C₁₋₆ alkyl C₁₋₆ alkoxy (which latter two groups are optionally substituted by one or more substituents selected from halo, OH, C₁₋₄ alkoxy, C(O)OR^{12a} and C(O)N(R^{12b})R^{12c}), S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent 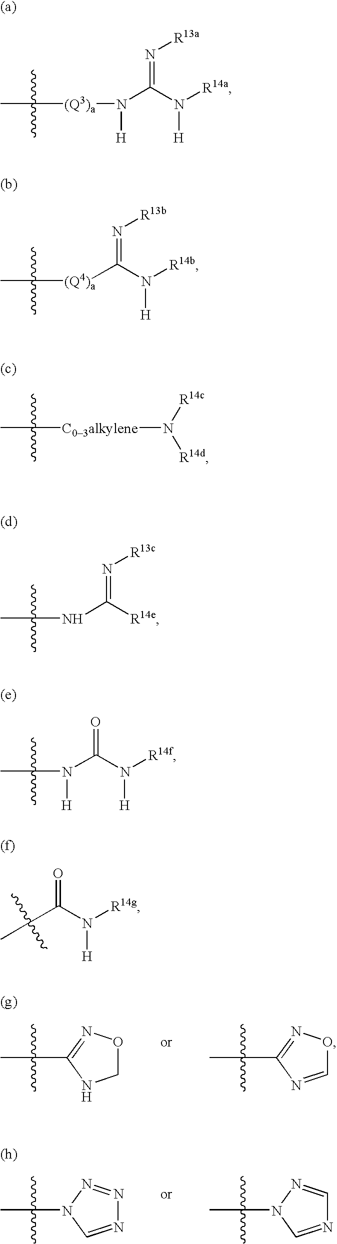
or R^{b} to R^{d} may also represent H;
except that R^{a}, R^{b}, R^{c} or R^{d} (as appropriate) is replaced by a cyano or —C(═NH)O—C₁₋₄ alkyl group;
Q³ represents O, N(R^{10c}), S(O)₂, S(O)—NH, C(O) or —CH═N—;
Q⁴ represents O, S or CH₂;
a represents 0 or 1;
R^{13a} to R^{13c} independently represent
(a) H,
(b) CN,
(c) NH₂,
(d) OR¹⁵ or
(e) C(O)OR¹⁶;
R¹⁵ represents
(a) H,
(b) C₁₋₁₀ alkyl C₃₋₁₀ alkenyl C₃₋₁₀ alkynyl,
(c) C₃₋₁₀ cycloalkyl, C₄₋₁₀ cycloalkenyl, which latter two groups are optionally substituted by one or more substituents selected from halo and C₁₋₆ alkyl, or
(d) C₁₋₃ alkyl, which latter group is optionally interrupted by oxygen and is substituted by aryl or —O-aryl,
R¹⁶ represents
(a) C₁₋₁₀ alkyl, C₃₋₁₀ alkenyl C₃₋₁₀ alkynyl, which latter three groups are optionally interrupted by one or more oxygen atoms, or
(b) C₃₋₁₀ cycloalkyl C₄₋₁₀ cycloalkenyl, which latter two groups are optionally substituted by one or more substituents selected from halo and C₁₋₆ alkyl, or
(c) C₁₋₃ alkyl, which latter group is optionally interrupted by oxygen and is substituted by aryl or —O-aryl,
R^{10c} and R^{14a} to R¹⁴, independently represent
(a) H or
(b) C₁₋₄ alkyl (which latter group is optionally substituted by one or more substituents selected from halo and OH),
or R^{14a} and R^{14b} independently represent C(O)O—C₁₋₆ alkyl (the alkyl part of which latter group is optionally substituted by aryl and/or one or more halo atoms),
or R^{14c} represents
(a) C₁₋₄ alkyl substituted by C₃₋₇ cycloalkyl or aryl,
(b) C₃₋₇ cycloalkyl,
(c) C(O)O—C₁₋₆ alkyl (the alkyl part of which latter group is optionally substituted by aryl and/or one or more halo atoms),
(d) C(O)C₁₋₆ alkyl,
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
or R^{14c} and R^{14d} together represent C₃₋₆ n-alkylene optionally interrupted by O, S, N(H) or N(C₁₋₄ alkyl) and/or substituted by one or more C₁₋₄ alkyl groups,
or a protected derivative thereof.

12. A compound of formula VI 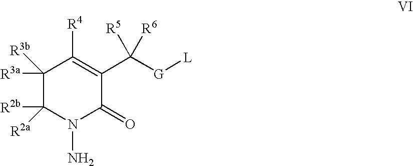
wherein
R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene,
R⁴ represents
(a) H,
(b) halo,
(c) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy C(O)OH, C(O)O—C₁₋₄ alkyl and OC(O)—C₁₋₄ alkyl),
(d) together with R⁵, R⁴ represents C₂₋₃ n-alkylene, T¹-(C₁₋₂ n-alkylene) or (C₁₋₂ n-alkylene)-T¹ which latter three groups are optionally substituted by halo, or
(e) together with R⁵ and R⁶, R⁴ represents T²-[C(H)═], wherein T is bonded to the C-atom to which the group R⁴ is attached;
R⁵ and R⁶ independently represent H, F or methyl (which latter group is optionally substituted by one or more F atoms), or
(a) together with R⁴, R⁵ represents C_{—-3} n-alkylene, T¹-(C₁₋₂ n-alkylene) or (C₁₋₂ n-alkylene)-T¹ which latter three groups are optionally substituted by halo, or
(b) together with R⁴, R⁵ and R⁶ represents T²-[C(H)═], wherein T is bonded to the C-atom to which the group R⁴ is attached;
T¹ and T² independently represent O, S, N(H) or N(C₁₋₄ alkyl); and
L represents
(a) C₀₋₆ alkylene-R^{a},
(b) C₀₋₂ alkylene-CH═CH—C₀₋₂ alkylene-R^{a},
(c) C₀₋₂ alkylene-C≡C—C₀₋₂alkylene-R^{a}, 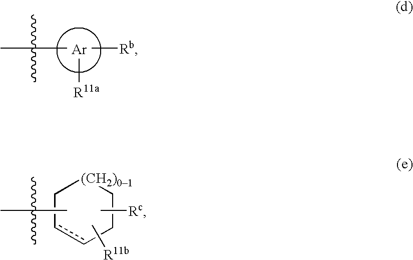
wherein the dashed line represents an optional double bond, or 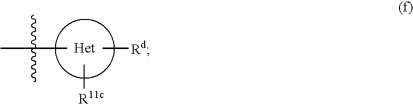
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl C₁₋₆ alkoxy (which latter two groups are optionally substituted by one or more substituents selected from halo, OH, C₁₋₄ alkoxy, C(O)OR^{12a} and C(O)N(R^{12b})R^{12c}) and S(O)₀₋₂R^{12d};
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}), S(O)₀₋₂R^{12d}, ═O, ═NH, ═NOH and ═N—CN;
R^{12a} to R^{12c} independently represent H, C₁₋₆ alkyl or C₁₋₆ cycloalkyl (which latter two groups are optionally substituted by one OH or N(R^{12e})R^{12f} group or by one or more halo atoms);
R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent 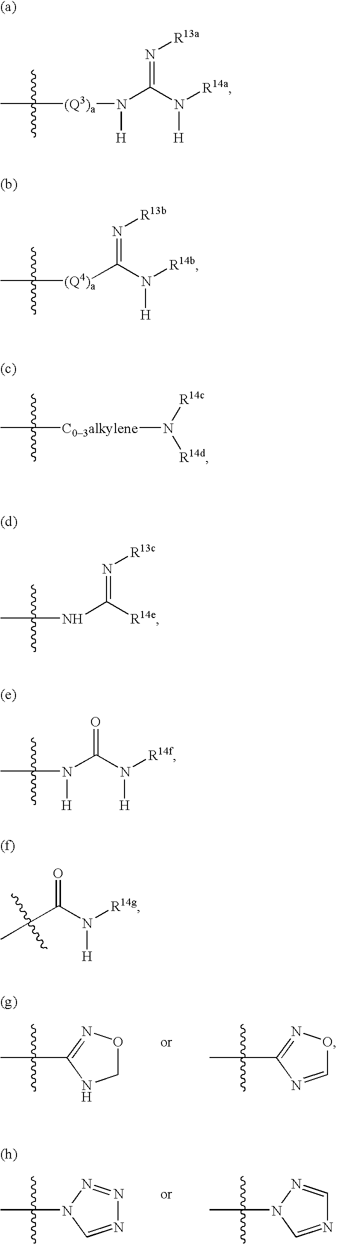
or R^{b} to R^{d} may also represent H;
Q³ represents O, N(R^{10c}), S(O)₂, S(O)₂NH, C(O) or —CH═N—;
Q⁴ represents O, S or CH₂;
a represents 0 or 1;
R^{13a} to R^{13c} independently represent
(a) H,
(b) CN
(c) NH₂,
(d) OR¹⁵ or
(e) C(O)OR¹⁶;
R¹⁵ represents
(a) H,
(b) C₁₋₁₀ alkyl C₃₋₁₀ alkenyl C₃₋₁₀ alkynyl,
(c) C₃₋₁₀ cycloalkyl C₄₋₁₀ cycloalkenyl, which latter two groups are optionally substituted by one or more substituents selected from halo and C₁₋₆ alkyl or (d) C₁₋₃ alkyl, which latter group is optionally interrupted by oxygen and is substituted by aryl or —O-aryl;
R¹⁶ represents
(a) C₁₋₁₀ alkyl C₃₋₁₀ alkenyl C₃₋₁₀ alkynyl, which latter three groups are optionally interrupted by one or more oxygen atoms, or
(b) C₃₋₁₀ cycloalkyl C₄₋₁₀ cycloalkenyl, which latter two groups are optionally substituted by one or more substituents selected from halo and C₁₋₆ alkyl or
(c) C₁₋₃ alkyl, which latter group is optionally interrupted by oxygen and is substituted by aryl or —O-aryl;
R^{10c} and R^{14a} to R^{14g} independently represent
(a) H or
(b) C₁₋₄ alkyl (which latter group is optionally substituted by one or more substituents selected from halo and OH),
or R^{14a} and R^{14b} independently represent C(O)O—C₁₋₆ alkyl (the alkyl part of which latter group is optionally substituted by aryl and/or one or more halo atoms),
or R^{14c} represents
(a) C₁₋₄ alkyl substituted by C₃₋₇ cycloalkyl or aryl,
(b) C₃₋₇ cycloalkyl,
(c) C(O)O—C₁₋₆ alkyl (the alkyl part of which latter group is optionally substituted by aryl and/or one or more halo atoms),
(d) C(O)C₁₋₆ alkyl
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
or R^{14c} and R^{14d} together represent C₃₋₆ n-alkylene optionally interrupted by O, S, N(H) or N(C₁₋₄ alkyl) and/or substituted by one or more C₁₋₄ alkyl groups,
G represents
(a) —C(O)N(R^{8a})—[CH(C(O)R⁹)]₀₋₁—C₀₋₃ alkylene-(Q¹)ₐ-,
(b) —C(O)N(R^{8b})—C₂₋₃ alkenylene-(Q¹)ₐ-, 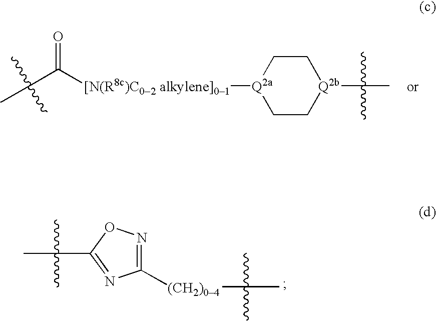
R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl,
Q¹ represents O, NR^{10a}, [N(H)]₀₋₁C(O)—C₀₋₂ alkylene, C(O)NHNHC(O), or —N═C(R^{10b})—;
a represents 0 or 1;
Q^{2a} represents 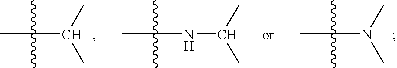
Q^{2b} represents 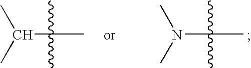
R^{8a} to R^{8c}, R^{10a} to R^{10b} independently represent
(a) H or
(b) C₁₋₄ alkyl (which latter group is optionally substituted by one or more substituents selected from halo and OH);
or a protected derivative thereof.
